(19) United States
(12) Patent Application Publication
(10) Pub. No.: US 2022/0331378 A1
(43) Pub. Date: Oct. 20, 2022

(54) COMPOSITIONS AND METHODS FOR TREATING AUTISM SPECTRUM DISORDER

(71) Applicant: Finch Therapeutics Holdings LLC, Somerville, MA (US)

(72) Inventors: Mark SMITH, Somerville, MA (US); Christopher WEIDENMAIER, Brookline, MA (US)

(73) Assignee: Finch Therapeutics Holdings LLC, Somerville, MA (US)

(21) Appl. No.: 17/642,520

(22) PCT Filed: Sep. 11, 2020
(86) PCT No.: PCT/US2020/050525

§ 371 Date: Mar. 11, 2022

Related U.S. Application Data
(60) Provisional application No. 62/950,805, filed on Dec. 19, 2019.
(60) Provisional application No. 62/899,874, filed on Sep. 13, 2019.

(51) Int. Cl. A61K 35/747; A61P 25/00

(52) U.S. Cl. CPC A61K 35/747; A61P 25/00; A61K 2300/00

(57) ABSTRACT

The present disclosure relates to compositions and methods for treating autism spectrum disorder (ASD). Provided herein are pharmaceutical compositions and formulations comprising a preparation of uncultured fecal bacteria derived from a stool of a human donor and at least one, at least two, or all three of non-pathogenic microbial types selected from the group consisting of a bacterial isolate, a fungal isolate, and an archaeal isolate (e.g., a bacterial isolate comprising Lactobacillus reuteri), and methods of treating an ASD patient with the composition. Further provided are methods of manufacturing a pharmaceutical composition comprising L. reuteri.

### CROSS-REFERENCE TO RELATED APPLICATION

This application claims the benefit of U.S. Provisional Application No. 62/950,805, filed Dec. 19, 2019, and U.S. Provisional Application No. 62/899,874, filed Sep. 13, 2019, which are incorporated by reference in their entireties herein.

### BACKGROUND

Autism spectrum disorder (ASD) is a complex neurodevelopmental condition characterized by widespread abnormalities of social interactions and communication, as well as restricted interests and repetitive behaviors. ASD typically appears during the first three years of life and manifests in characteristic symptoms or behavioral traits. A diagnosis of ASD now includes several conditions that used to be diagnosed separately: autistic disorder, pervasive developmental disorder not otherwise specified (PDD-NOS), and Asperger syndrome. All of these conditions are now encompassed by the diagnostic criteria for autism spectrum disorder as set forth in the American Psychiatric Association's Diagnostic & Statistical Manual of Mental Disorders, Fifth Edition (DSM-V).

In addition to the spectrum of symptoms seen within these principal diagnostic criteria, ASD individuals display a wide range of neurological comorbidities, including intellectual disability, epilepsy, and anxiety and mood disorders, as well as non-neurological comorbidities, including blood hyperserotonemia, immune dysregulation, and GI dysfunction (e.g., chronic constipation, diarrhea, abdominal pain, and gastroesophageal reflux).

Mammals harbor diverse microbial species in their gastrointestinal (GI) tracts. Interactions between these microbes and between microbes and the host, e.g. the host immune system, shape a microbiota. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. An unbalanced microbiota (also called ‘dysbiosis’ or disrupted symbiosis) may lose its function and results in increased susceptibility to pathogens, altered metabolic profiles, or induction of proinflammatory signals that can lead to local or systemic inflammation or autoimmunity. Additionally, such a disrupted microbiota may be infected by incoming pathogen or pathogens, which can cause pain, diarrhea, gas, and constipation among other symptoms. Hence, the intestinal microbiota plays a significant role in the pathogenesis of many disorders such as pathogenic infections of the gut.

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

FMT has been used to treat Clostridium difficile infection (CDI). FMT has also been suggested in treating other gut infective agents such as E. coli and Vancomycin resistant Enterococci (VRE). It entails infusions through a colonoscope, an enema or via a nasojejunal tube of human microbiota either in the form of homogenized stool, or cultured stool components such as Clostridia, to implant in the colon and thereby displace or eradicate pathogenic bacteria, e.g., C. difficile. Fecal bacteriotherapy has also been successful in treating conditions having a neurological component, such as ASD, Parkinson's Disease, and Multiple Sclerosis and Chronic Fatigue Syndrome.

In at least some cases, the precise mechanism by which fecal bacteriotherapy acts to treat a condition, for example ASD, is unknown. For example, a typical donor fecal microbiota administered to a patient during fecal bacteriotherapy can contain hundreds of bacterial strains, and the identity of the strains necessary for the treatment of ASD, as well as the mechanisms by which such introduced strains interact with each other and the bacteria of the patient's endogenous microbiome, is largely unknown. Further, potential variation in (i) the identity and relative abundance of particular bacterial strains across different donor samples; and (ii) the degree to which a particular bacterial strain engrafts in the intestine of a bacteriotherapy recipient, can lead to uncertainty regarding efficacy of fecal bacteriotherapy across a patient group afflicted with or susceptible to a disorder.

### SUMMARY

In an aspect, this disclosure provides for a pharmaceutical composition comprising a bacterial mixture comprising: (i) a preparation of uncultured fecal bacteria derived from a stool of a human donor; and (ii) a bacterial isolate comprising Lactobacillus reuteri.

In another aspect, this disclosure provides for a pharmaceutical composition comprising a bacterial mixture comprising: (i) a preparation of uncultured fecal bacteria derived from a stool of a human donor; and (ii) a bacterial isolate comprising a species of Lactobacillus; wherein the preparation of uncultured fecal bacteria does not comprise the species of the genus Lactobacillus.

In another aspect, this disclosure provides for a pharmaceutical composition comprising a bacterial mixture comprising: (i) a preparation of uncultured fecal bacteria derived from a stool of a human donor; and (ii) a non-pathogenic bacterial isolate, wherein a relative abundance of viable cells of the bacterial isolate in the bacterial mixture is at least 10%.

In another aspect, this disclosure provides for a pharmaceutical composition comprising a bacterial mixture comprising: (i) a preparation of uncultured fecal bacteria derived from a stool of a human donor; and (ii) a non-pathogenic bacterial isolate; wherein a relative abundance of viable cells of the bacterial isolate in the bacterial mixture is greater than a relative abundance of viable cells of any bacterial strain in the preparation of uncultured fecal bacteria.

In another aspect, this disclosure provides for a pharmaceutical composition comprising a bacterial mixture comprising: (i) a preparation of uncultured fecal bacteria derived from a stool of a human donor; and (ii) a non-pathogenic bacterial isolate; wherein the bacterial isolate is a member of a species, wherein the bacterial isolate is the only member of the species in the bacterial mixture.

In another aspect, this disclosure provides for a pharmaceutical composition comprising a bacterial mixture comprising: (i) a preparation of uncultured fecal bacteria derived from a stool of a human donor; and (ii) a non-pathogenic bacterial isolate; wherein the preparation of uncultured fecal bacteria does not comprise a bacterial strain having a 16S rRNA sequence greater than 99% identical to a 16S rRNA sequence of the bacterial isolate.

In another aspect, this disclosure provides for a pharmaceutical composition comprising a bacterial mixture comprising: (i) a preparation of uncultured fecal bacteria derived from a stool of a human donor; and (ii) a non-pathogenic bacterial isolate; wherein the bacterial isolate engrafts in the ileum of a subject administered the composition.

In another aspect, this disclosure provides for a method for treating at least one symptom of an autism spectrum disorder (ASD) in a subject in need thereof, the method comprising administering to the subject (i) a pharmaceutical composition comprising a bacterial population or community derived from a stool of a human donor, wherein the bacterial population or community is not cultured; and (ii) a bacterial isolate comprising Lactobacillus reuteri.

In another aspect, this disclosure provides for a method for engrafting Lactobacillus reuteri in an intestine of a human, the method comprising administering to the human a pharmaceutical composition comprising (i) a preparation of uncultured fecal bacteria; and (ii) a bacterial isolate comprising L. reuteri; wherein a relative abundance of L. reuteri in an intestinal microbiota of the human after administering the composition is greater than a relative abundance of L. reuteri in the intestinal microbiota prior to administering the composition.

In another aspect, this disclosure provides for a method comprising: extracting a bacterial population or community from a stool of a healthy human donor; and mixing the bacterial population or community with a bacterial isolate comprising Lactobacillus reuteri; wherein the bacterial population or community is not cultured.

In yet another aspect, this disclosure provides for a method comprising: selecting a human stool donor based on an abundance of at least one member of Lactobacillus in a fecal microbiota of the donor; extracting a population or community of bacteria from a stool of the donor, wherein the population or community of bacteria comprises the at least one member of Lactobacillus; and incorporating the population or community of bacteria into a pharmaceutical composition, wherein the population or community of bacteria is not cultured.

In a further aspect, this disclosure provides for a method of manufacturing a pharmaceutical composition, the method comprising: extracting a bacterial population or community from a stool of a healthy human donor; and incorporating the extracted bacterial population or community into the pharmaceutical composition, wherein the bacterial population or community comprises a bacterial strain originating from a probiotic ingested by the healthy human donor.

In another aspect, this disclosure provides for a method of manufacturing a pharmaceutical composition comprising a bacterial population or community of a healthy human donor, the method comprising: receiving a stool from the donor following ingestion by the donor of a probiotic comprising a bacterial strain; extracting the bacterial population or community from the stool, wherein the bacterial population or community comprises the bacterial strain; incorporating the bacterial population or community into the pharmaceutical composition, wherein the bacterial population or community is not cultured; and wherein prior to ingestion of the probiotic by the donor, a stool of the donor did not comprise the bacterial strain.

In another aspect, this disclosure provides for a pharmaceutical composition comprising a mixture comprising: (i) a preparation of uncultured fecal bacteria derived from a stool of a human donor; and (ii) a non-pathogenic fungal isolate, wherein a relative abundance of viable cells of the fungal isolate in the mixture is at least 10%.

In another aspect, this disclosure provides for a pharmaceutical composition comprising a mixture comprising: (i) a preparation of uncultured fecal bacteria derived from a stool of a human donor; and (ii) a non-pathogenic fungal isolate; wherein a relative abundance of viable cells of the fungal isolate in the mixture is greater than a relative abundance of viable cells of any bacterial strain in the preparation of uncultured fecal bacteria.

In another aspect, this disclosure provides for a pharmaceutical composition comprising a mixture comprising: (i) a preparation of uncultured fecal bacteria derived from a stool of a human donor; and (ii) a non-pathogenic fungal isolate; wherein the fungal isolate is a member of a species, wherein the fungal isolate is the only member of the species in the mixture.

In another aspect, this disclosure provides for a pharmaceutical composition comprising a mixture comprising: (i) a preparation of uncultured fecal microbes derived from a stool of a human donor; and (ii) a non-pathogenic fungal isolate; wherein the preparation of uncultured fecal microbes does not comprise a fungal strain having a 16S rRNA sequence greater than 99% identical to a 16S rRNA sequence of the fungal isolate.

In another aspect, this disclosure provides for a pharmaceutical composition comprising a mixture comprising: (i) a preparation of uncultured fecal bacteria derived from a stool of a human donor; and (ii) a non-pathogenic fungal isolate; wherein the fungal isolate engrafts in the ileum of a subject administered the composition.

In another aspect, this disclosure provides for a method for treating at least one symptom of an autism spectrum disorder (ASD) in a subject in need thereof, the method comprising administering to the subject (i) a pharmaceutical composition comprising a bacterial population or community derived from a stool of a human donor, wherein the bacterial population or community is not cultured; and (ii) a fungal isolate.

In another aspect, this disclosure provides for a method comprising: extracting a bacterial population or community from a stool of a healthy human donor; and mixing the bacterial population or community with a fungal isolate; wherein the bacterial population or community is not cultured.

In yet another aspect, this disclosure provides for a method comprising: selecting a human stool donor based on an abundance of at least one fungal member in a fecal microbiota of the donor; extracting a population or community of microbes from a stool of the donor, wherein the population or community of microbes comprises the at least one fungal member; and incorporating the population or community of microbes into a pharmaceutical composition, wherein the population or community of microbes is not cultured.

In a further aspect, this disclosure provides for a method of manufacturing a pharmaceutical composition, the method comprising: extracting a microbial population or community from a stool of a healthy human donor; and incorporating the extracted microbial population or community into the pharmaceutical composition, wherein the microbial population or community comprises a fungal strain originating from a probiotic ingested by the healthy human donor.

In another aspect, this disclosure provides for a method of manufacturing a pharmaceutical composition comprising a microbial population or community of a healthy human donor, the method comprising: receiving a stool from the donor following ingestion by the donor of a probiotic comprising a fungal strain; extracting the microbial population or community from the stool, wherein the microbial population or community comprises the fungal strain; incorporating the microbial population or community into the pharmaceutical composition, wherein the microbial population or community is not cultured; and wherein prior to ingestion of the probiotic by the donor, a stool of the donor did not comprise the fungal strain.

In another aspect, this disclosure provides for a pharmaceutical composition comprising a mixture comprising: (i) a preparation of uncultured fecal bacteria derived from a stool of a human donor; (ii) a non-pathogenic bacterial isolate; and (iii) a non-pathogenic fungal isolate; wherein a relative abundance of viable cells of the bacterial isolate and/or a relative abundance of viable cells of the fungal isolate in the mixture are at least 10%.

In another aspect, this disclosure provides for a pharmaceutical composition comprising a mixture comprising: (i) a preparation of uncultured fecal bacteria derived from a stool of a human donor; (ii) a non-pathogenic bacterial isolate; and (iii) a non-pathogenic fungal isolate; wherein a relative abundance of viable cells of the bacterial isolate and/or the fungal isolate in the mixture is greater than a relative abundance of viable cells of any bacterial strain in the preparation of uncultured fecal bacteria.

In another aspect, this disclosure provides for a pharmaceutical composition comprising a mixture comprising: (i) a preparation of uncultured fecal bacteria derived from a stool of a human donor; (ii) a non-pathogenic bacterial isolate; and (iii) a non-pathogenic fungal isolate; wherein the bacterial isolate is a member of a first species, wherein the bacterial isolate is the only member of the first species in the mixture, wherein the fungal isolate is a member of a second species, wherein the fungal isolate is the only member of the second species in the mixture.

In another aspect, this disclosure provides for a pharmaceutical composition comprising a mixture comprising: (i) a preparation of uncultured fecal microbes derived from a stool of a human donor; (ii) a non-pathogenic bacterial isolate; and (iii) a non-pathogenic fungal isolate; wherein the preparation of uncultured fecal microbes does not comprise a bacterial strain having a 16S rRNA sequence greater than 99% identical to a 16S rRNA sequence of the bacterial isolate, wherein the preparation of uncultured fecal microbes does not comprise a fungal strain having a 16S rRNA sequence greater than 99% identical to a 16S rRNA sequence of the fungal isolate.

In another aspect, this disclosure provides for a pharmaceutical composition comprising a mixture comprising: (i) a preparation of uncultured fecal bacteria derived from a stool of a human donor; (ii) a non-pathogenic bacterial isolate; and (iii) a non-pathogenic fungal isolate; wherein the bacterial isolate and fungal isolate engrafts in the ileum of a subject administered the composition.

In another aspect, this disclosure provides for a method for treating at least one symptom of an autism spectrum disorder (ASD) in a subject in need thereof, the method comprising administering to the subject (i) a pharmaceutical composition comprising a bacterial population or community derived from a stool of a human donor, wherein the bacterial population or community is not cultured; and (ii) a non-pathogenic bacterial isolate; and (iii) a non-pathogenic fungal isolate.

In another aspect, this disclosure provides for a method comprising: extracting a bacterial population or community from a stool of a healthy human donor; and mixing the bacterial population or community with (i) a non-pathogenic bacterial isolate and (ii) a non-pathogenic fungal isolate; wherein the bacterial population or community is not cultured.

In yet another aspect, this disclosure provides for a method comprising: selecting a human stool donor based on an abundance of at least one bacterial member and/or at least one fungal member in a fecal microbiota of the donor; extracting a population or community of microbes from a stool of the donor, wherein the population or community of microbes comprises the at least one bacterial member and/or at least one fungal member; and incorporating the population or community of microbes into a pharmaceutical composition, wherein the population or community of microbes is not cultured.

In a further aspect, this disclosure provides for a method of manufacturing a pharmaceutical composition, the method comprising: extracting a microbial population or community from a stool of a healthy human donor; and incorporating the extracted microbial population or community into the pharmaceutical composition, wherein the microbial population or community comprises a bacterial strain and/or a fungal strain originating from a probiotic ingested by the healthy human donor.

In another aspect, this disclosure provides for a method of manufacturing a pharmaceutical composition comprising a microbial population or community of a healthy human donor, the method comprising: receiving a stool from the donor following ingestion by the donor of a probiotic comprising a bacterial strain and/or a fungal strain; extracting the microbial population or community from the stool, wherein the microbial population or community comprises the bacterial strain and/or fungal strain; incorporating the microbial population or community into the pharmaceutical composition, wherein the microbial population or community is not cultured; and wherein prior to ingestion of the probiotic by the donor, a stool of the donor did not comprise the bacterial strain and/or the fungal strain.

In another aspect, this disclosure provides for a pharmaceutical composition comprising a mixture comprising: (i) a preparation of uncultured fecal bacteria derived from a stool of a human donor; and (ii) at least one, at least two, or all three of non-pathogenic microbial types selected from the group consisting of a bacterial isolate, a fungal isolate, and an archaeal isolate; wherein a relative abundance of viable cells of the at least one, at least two, or all three of non-pathogenic microbe types in the mixture is at least 10%.

In another aspect, this disclosure provides for a pharmaceutical composition comprising a mixture comprising: (i) a preparation of uncultured fecal bacteria derived from a stool of a human donor; and (ii) at least one, at least two, or all three of non-pathogenic microbial types selected from the group consisting of a bacterial isolate, a fungal isolate, and an archaeal isolate; wherein a relative abundance of viable cells of the at least one, at least two, or all three of non-pathogenic microbe types in the mixture is greater than a relative abundance of viable cells of any bacterial strain in the preparation of uncultured fecal bacteria.

In another aspect, this disclosure provides for a pharmaceutical composition comprising a mixture comprising: (i) a preparation of uncultured fecal bacteria derived from a stool of a human donor; and (ii) at least one, at least two, or all three of non-pathogenic microbial types selected from the group consisting of a bacterial isolate, a fungal isolate, and an archaeal isolate; wherein the bacterial isolate is a member of a first species, wherein the bacterial isolate is the only member of the first species in the mixture, wherein the fungal isolate is a member of a second species, wherein the fungal isolate is the only member of the second species in the mixture, wherein the archaeal isolate is a member of a third species, wherein the archaeal isolate is the only member of the third species in the mixture.

In another aspect, this disclosure provides for a pharmaceutical composition comprising a mixture comprising: (i) a preparation of uncultured fecal microbes derived from a stool of a human donor; and (ii) at least one, at least two, or all three of non-pathogenic microbial types selected from the group consisting of a bacterial isolate, a fungal isolate, and an archaeal isolate; wherein the preparation of uncultured fecal microbes does not comprise a bacterial strain having a 16S rRNA sequence greater than 99% identical to a 16S rRNA sequence of the bacterial isolate, wherein the preparation of uncultured fecal microbes does not comprise a fungal strain having a 16S rRNA sequence greater than 99% identical to a 16S rRNA sequence of the fungal isolate, wherein the preparation of uncultured fecal microbes does not comprise a archaeal strain having a 16S rRNA sequence greater than 99% identical to a 16S rRNA sequence of the archaeal isolate.

In another aspect, this disclosure provides for a pharmaceutical composition comprising a mixture comprising: (i) a preparation of uncultured fecal bacteria derived from a stool of a human donor; and (ii) at least one, at least two, or all three of non-pathogenic microbial types selected from the group consisting of a bacterial isolate, a fungal isolate, and an archaeal isolate; wherein the at least one, at least two, or all three of non-pathogenic microbial types engraft in the ileum of a subject administered the composition.

In another aspect, this disclosure provides for a method for treating at least one symptom of an autism spectrum disorder (ASD) in a subject in need thereof, the method comprising administering to the subject (i) a pharmaceutical composition comprising a bacterial population or community derived from a stool of a human donor, wherein the bacterial population or community is not cultured; and (ii) at least one, at least two, or all three of non-pathogenic microbial types selected from the group consisting of a bacterial isolate, a fungal isolate, and an archaeal isolate.

In another aspect, this disclosure provides for a method comprising: extracting a bacterial population or community from a stool of a healthy human donor; and mixing the bacterial population or community with (i) a non-pathogenic bacterial isolate and (ii) at least one, at least two, or all three of non-pathogenic microbial types selected from the group consisting of a bacterial isolate, a fungal isolate, and an archaeal isolate; wherein the bacterial population or community is not cultured.

In yet another aspect, this disclosure provides for a method comprising: selecting a human stool donor based on an abundance of at least one member in a fecal microbiota of the donor; extracting a population or community of microbes from a stool of the donor, wherein the population or community of microbes comprises the at least one member; and incorporating the population or community of microbes into a pharmaceutical composition, wherein the population or community of microbes is not cultured, wherein the at least one member comprises at least one, at least two, or all three of non-pathogenic microbial types selected from the group consisting of a bacterial isolate, a fungal isolate, and an archaeal isolate.

In a further aspect, this disclosure provides for a method of manufacturing a pharmaceutical composition, the method comprising: extracting a microbial population or community from a stool of a healthy human donor; and incorporating the extracted microbial population or community into the pharmaceutical composition, wherein the microbial population or community comprises at least one member originating from a probiotic ingested by the healthy human donor, wherein the at least one member comprises at least one, at least two, or all three of non-pathogenic microbial types selected from the group consisting of a bacterial isolate, a fungal isolate, and an archaeal isolate.

In another aspect, this disclosure provides for a method of manufacturing a pharmaceutical composition comprising a microbial population or community of a healthy human donor, the method comprising: receiving a stool from the donor following ingestion by the donor of a probiotic comprising at least one, at least two, or all three of non-pathogenic microbial types selected from the group consisting of a bacterial isolate, a fungal isolate, and an archaeal isolate; extracting the microbial population or community from the stool, wherein the microbial population or community comprises the at least one, at least two, or all three of non-pathogenic microbial types; incorporating the microbial population or community into the pharmaceutical composition, wherein the microbial population or community is not cultured; and wherein prior to ingestion of the probiotic by the donor, a stool of the donor did not comprise microbes of the at least one, at least two, or all three of non-pathogenic microbial types.

### BRIEF DESCRIPTION OF THE DRAWINGS

FIG. 1: Graph showing a variable relative abundance of Lactobacillus reuteri in fecal microbiota of healthy stool donors.

FIG. 2: PBMC stimulation assays to show immune-stimulating effects from non-selected donor-derived fecal microbiota (FSM), alone or in combination with selected bacterial isolates, measured by the level of IFN-gamma.

FIG. 3: PBMC stimulation assays to show immune-stimulating effects from non-selected donor-derived fecal microbiota (FSM), alone or in combination with selected bacterial isolates, measured by the level of IL-12p70.

FIG. 4: PBMC stimulation assays to show immune-stimulating effects from non-selected donor-derived fecal microbiota (FSM), alone or in combination with selected bacterial isolates, measured by the level of IL-23.

FIG. 5: PBMC stimulation assays to show immune-stimulating effects from non-selected donor-derived fecal microbiota (FSM), alone or in combination with selected bacterial isolates, measured by the level of GM-CSF.

FIG. 6: PBMC stimulation assays to show immune-stimulating effects from non-selected donor-derived fecal microbiota (FSM), alone or in combination with selected bacterial isolates, measured by the ratio between IL-10 and IL-12p70.

FIG. 7: A first trial design to treat ASD using a donor-derived bacterial composition combined with an bacterial strain of interest.

FIG. 8: A second trial design to treat ASD using a donor-derived bacterial composition combined with an bacterial strain of interest.

### DETAILED DESCRIPTION

Unless defined otherwise, all technical and scientific terms used herein have the same meaning as commonly understood by one of ordinary skill in the art to which this disclosure belongs.

All publications, patents, and patent applications mentioned in this specification are herein incorporated by reference to the same extent as if each individual publication, patent, or patent application was specifically and individually indicated to be incorporated by reference.

As used herein and in the appended claims, the singular forms “a,” “an,” and “the” include plural referents unless the context clearly dictates otherwise. By way of example, “an element” means at least one element and can include more than one element.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5% or 10%.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure.

To avoid any doubt, used herein, terms or phrases such as “about”, “at least”, “at least about”, “at most”, “less than”, “greater than”, “within” or alike, when followed by a series of list of numbers of percentages, such terms or phrases are deemed to modify each and every number of percentage in the series or list, regardless whether the adverb, preposition, or other modifier phrase is reproduced prior to each and every member.

As used herein, the term “relative abundance” refers to relative representation of an organism of a particular kind (e.g., a bacterial strain, species, or genus) relative to all organisms of similar nature in a certain community (e.g., a preparation of uncultured fecal bacteria or a bacterial mixture). Relative abundance is calculated by dividing the number of an organism of a particular kind by the total number of all organisms of similar nature in a certain community. In an aspect, relative abundance is measured by qPCR comparing PCR products generated with 16S primers targeting specific bacterial strains of interest against PCR products generated with universal primers targeting all 16S sequences. See e.g., Chu, N., et al., “Profiling living bacteria informs preparation of fecal microbiota transplantations.” PLoS One 12(1): 1-16 (2017). In another aspect, the relative abundance is measured based on the number of sequence reads detected via high-throughput sequencing as described in Gevers et al., “The treatment-naïve microbiomes in new-onset Crohn's disease.” Cell Host & Microbe, 15(3):382-92(2014). In an aspect, high-throughput sequencing is based on 16S rRNA gene sequencing. In another aspect, high-throughput sequencing is based on whole-genome short-gun metagenomic sequencing. Unless specified otherwise, a bacterial relative abundance mentioned herein is measured via high-throughput sequencing of 16S rRNA targeting the V4 variable region as described in Gevers et al., Cell Host & Microbe, 15(3):382-92(2014). In a further aspect, propidium monoazide (PMA) is used to differentiate between viable and dead fecal microbes as shown in Chu et al., PLoS One 12(1): 1-16 (2017).

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. In the context of autism spectrum disorder, “treat” and “treating” encompass alleviating, ameliorating, delaying the onset of, inhibiting the progression of, or reducing the severity of one or more symptoms associated with an autism spectrum disorder.

As used herein, a “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). Preferred subjects are human subjects. The human subject may be a pediatric, adult or a geriatric subject. In some aspects, the terms “patient” and “subject” are used interchangeably. The subject may be healthy, or may be suffering from one or more symptoms of ASD.

As used herein, a “microbiota” and “flora” refer to a community of microbes that live in or on a subject's body, both sustainably and transiently, including eukaryotes, archaea, bacteria, and viruses (including bacterial viruses (i.e., phage)). A “fecal microbiota” or “fecal microbiota preparation” refers to a community of microbes present in or prepared from a subject's feces. Typically a pharmaceutical composition described herein is prepared by incorporating such a fecal microbiota into the composition without culturing the fecal microbiota after its purification from a stool. Herein a “preparation of uncultured fecal bacteria” refers to multiple viable bacterial strains that have been harvested, extracted or purified from one or more stool samples, without culturing the strains (e.g. in culturing medium).

In some aspects, a preparation of uncultured fecal bacteria comprises non-selected fecal bacteria. Herein “non-selected fecal bacteria” refers to a population or community of viable fecal bacterial strains (e.g., present in a fecal microbiota) extracted from one or more stool samples without subjecting the extracted population or community to environmental conditions that intentionally select for a particular type, state or taxonomic category of bacteria (e.g., by deliberate removal of certain strains of bacteria, treatment of the population or community with an agent such as ethanol or chloroform, or culturing). Such non-selected fecal bacteria can comprise bacterial strains in proportional content to corresponding bacterial strains in a fecal or intestinal microbiota of a normal healthy human. Steps taken to non-selectively extract a population or community of fecal bacteria from a stool sample can include, for example, homogenization and filtering of the stool sample to separate the fecal bacterial strains from non-cellular stool material such as fiber and rough particulate matter, as well as, for example, eukaryotic host cells and viruses. Herein typically a non-selected fecal bacterial preparation can be prepared in either aerobic or anaerobic conditions, or a combination thereof. In certain aspects, a preparation of non-selected fecal bacteria comprises all or substantially all of the bacteria of a fecal microbiota of a stool sample. In certain aspects, a preparation of non-selected fecal bacteria comprises all or substantially all of the strains of a fecal microbiota of a stool sample. In certain aspects, a preparation of non-selected fecal bacteria comprises all or substantially all of the species of a fecal microbiota of a stool sample. In certain aspects, a preparation of non-selected fecal bacteria comprises all or substantially all of the genera of a fecal microbiota of a stool sample. In certain aspects, a preparation of non-selected fecal bacteria comprises all or substantially all of the phyla of a fecal microbiota of a stool sample. Therefore, such non-selective fecal microbiota can substantially resemble microbial constituents and the bacterial population or community structure found in such fecal sample.

In an aspect, an uncultured bacterial population or community comprises at least 2, 5, 10, 20, 30, 40, 50, 100, 200, 300, 400, 500, or 600 bacterial species or strains. In another aspect, an uncultured bacterial population or community comprises between 2 and 5, 5 and 10, 10 and 20, 20 and 30, 30 and 40, 40 and 50, 50 and 60, 60 and 100, 100 and 200, 200 and 300, 300 and 400, 400 and 500, or 500 and 600 bacterial species or strains.

In an aspect, a preparation of uncultured fecal bacteria and/or non-selected fecal bacteria does not comprise an antibiotic resistant population of bacteria.

In another aspect, manufacture of a preparation of uncultured fecal bacteria can involve steps that select for a particular, type, state, or taxonomic category of bacteria (e.g., by deliberate removal of certain strains of bacteria and/or treatment of the population with a selective agent such as ethanol or chloroform). In certain aspects, such a preparation of uncultured fecal bacteria can be combined with one or more bacterial isolates to form a bacterial mixture for incorporation into a pharmaceutical composition. For example, a stool, or fecal bacteria extracted from a stool, can be incubated with a selective agent such as ethanol for a period of time, the ethanol removed after the incubation, and the incubated bacteria mixed with one or more bacterial isolates to produce a bacterial mixture. In an aspect, the viable bacteria remaining in a preparation after the incubation with the selective agent substantially comprise spores, or consist of spores.

Herein a preparation of uncultured fecal bacteria is distinguished from a single, purified strain of bacteria such as a bacterial isolate. As used herein, “bacterial isolate” refers to an isolated group of substantially genetically identical bacterial cells generated by proliferation via binary fission from a single predecessor bacterial cell (e.g., by culturing the bacteria). Typically, a bacterial isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. Once isolated, in some aspects, a bacterial isolate can be grown as a pure culture of cells; in other aspects, multiple bacterial isolates can be grown simultaneously in the same vessel as a mixed culture. In the bacterial context, the term “substantially genetically identical” refers to the very high (e.g. >99.9%) genetic identity shared by different cells in uncontaminated pure compositions of bacterial isolates, owing to their proliferation from a common predecessor, but accounts for minor genetic dissimilarity between cells due to accumulations of relatively rare mutations. Generally, a bacterial isolate is synonymous with a pure culture of bacterial cells. Typically, herein a bacterial isolate consists of non-pathogenic bacteria. In an aspect, a bacterial isolate can be a probiotic, or an ingredient in a probiotic.

As used herein, the term “bacterial cocktail”, sometimes called a “bacterial consortium” or “synthetic bacterial mixture”, refers to an engineered mixture of bacteria comprising a defined consortium of multiple bacterial isolates. The term “defined consortium of multiple bacterial isolates” means that the bacterial cocktail contains two or more bacterial isolates, and that the identity of each bacterial isolate in the cocktail is known, and thus the cocktail can be consistently produced (e.g. by combining isolated bacterial strains) to have a stable composition and properties across separate batches. Herein “identity” of a bacterial isolate can refer to any characteristic of the isolate that uniquely identifies the isolate as different from one or more other bacterial isolates or bacterial strains. Examples of identifying characteristics of a bacterial isolate include nucleotide sequences such as a 16S rRNA sequence, the sequence of one or more coding or non-coding regions of a nucleic acid, and entire genome sequences, levels of gene expression, physiological or metabolic traits, or anatomical traits such as staining pattern or cell wall characteristics.

As used herein, “bacterial mixture” refers to an engineered composition comprising viable bacterial cells, which in some aspects can include one or more non-pathogenic bacterial isolates and/or a preparation of uncultured bacterial cells. In some aspects, a bacterial mixture comprises one or more non-pathogenic bacterial isolates. In some aspects, a bacterial mixture comprises a preparation of uncultured fecal bacteria. In some aspects, a bacterial mixture comprises both of one or more non-pathogenic bacterial isolates and a preparation of uncultured fecal bacteria.

As used herein, “fungal isolate” refers to an isolated group of substantially genetically identical fungal cells generated by proliferation via binary fission from a single predecessor fungal cell (e.g., by culturing the fungi). Typically, a fungal isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. Once isolated, in some aspects, a fungal isolate can be grown as a pure culture of cells; in other aspects, multiple fungal isolates can be grown simultaneously in the same vessel as a mixed culture. In the fungal context, the term “substantially genetically identical” refers to the very high (e.g. >99.9%) genetic identity shared by different cells in uncontaminated pure compositions of fungal isolates, owing to their proliferation from a common predecessor, but accounts for minor genetic dissimilarity between cells due to accumulations of relatively rare mutations. Generally, a fungal isolate is synonymous with a pure culture of fungal cells. Typically, herein a fungal isolate consists of non-pathogenic fungi. In an aspect, a fungal isolate can be a probiotic, or an ingredient in a probiotic.

Any aspect described herein referencing or related to a bacterial isolate can be equally applicable to a fungal isolate. For example, all the disclosure and description herein about a mixture comprising a preparation of uncultured fecal bacteria enriched, supplemented or “spiked” with one or more bacterial isolates applies equally to a mixture comprising a preparation of uncultured fecal bacteria enriched, supplemented or “spiked” with one or more fungal isolates. Further for example, all the disclosure and description herein about a mixture comprising a preparation of uncultured fecal bacteria enriched, supplemented or “spiked” with one or more bacterial isolates applies equally to a mixture comprising a preparation of uncultured fecal bacteria enriched, supplemented or “spiked” with one or more bacterial isolates and one or more fungal isolates.

As used herein, “archaeal isolate” refers to an isolated group of substantially genetically identical archaeal cells generated by proliferation via binary fission from a single predecessor archaeal cell (e.g., by culturing the archaea). Typically, an archaeal isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. Once isolated, in some aspects, an archaeal isolate can be grown as a pure culture of cells; in other aspects, multiple archaeal isolates can be grown simultaneously in the same vessel as a mixed culture. In the archaeal context, the term “substantially genetically identical” refers to the very high (e.g. >99.9%) genetic identity shared by different cells in uncontaminated pure compositions of archaeal isolates, owing to their proliferation from a common predecessor, but accounts for minor genetic dissimilarity between cells due to accumulations of relatively rare mutations. Generally, an archaeal isolate is synonymous with a pure culture of archaeal cells. Typically, herein an archaeal isolate consists of non-pathogenic archaea. In an aspect, an archaeal isolate can be a probiotic, or an ingredient in a probiotic.

Any aspect described herein referencing or related to a bacterial isolate can also be equally applicable to an archaeal isolate. For example, all the disclosure and description herein about a mixture comprising a preparation of uncultured fecal bacteria enriched, supplemented or “spiked” with one or more bacterial isolates applies equally to a mixture comprising a preparation of uncultured fecal bacteria enriched, supplemented or “spiked” with one or more archaeal isolates (and/or one or more fungal isolates). Further for example, all the disclosure and description herein about a mixture comprising a preparation of uncultured fecal bacteria enriched, supplemented or “spiked” with one or more bacterial isolates applies equally to a mixture comprising a preparation of uncultured fecal bacteria enriched, supplemented or “spiked” with one or more bacterial isolates and one or more archaeal isolates. Similarly, all the disclosure and description herein about a mixture comprising a preparation of uncultured fecal bacteria enriched, supplemented or “spiked” with one or more bacterial isolates applies equally to a mixture comprising a preparation of uncultured fecal bacteria enriched, supplemented or “spiked” with one or more bacterial isolates, one or more archaeal isolates, and one or more archaeal isolates.

As used herein, “therapeutically effective amount,” “effective amount” or “pharmaceutically active dose” refers to an amount of a composition which is effective in treating the named disease, disorder, condition, or symptom.

As used herein, “isolated” or “purified” refers to a bacterium or other entity or substance that has been (1) separated from at least some of the components with which it was associated when initially produced (whether it was initially produced in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, the terms “non-pathogenic” in reference to a bacterium or any other organism or entity includes any such organism or entity that is not capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, “colony forming units” (CFUs) refers to an estimate of the number of viable microorganism cells in a given sample. The number of CFUs can be assessed by counting the number of colonies on an agar plate as in standard methods for determining the number of viable bacterial cells in a sample.

As used herein, “viable” means possessing the ability to multiply. The viability of bacterial populations can be monitored as a function of the membrane integrity of the cell. Cells with a compromised membrane are considered to be dead or dying, whereas cells with an intact membrane are considered live. For example, SYTO 9 and propidium iodide are used to stain and differentiate live and dead bacteria. See Stocks, Cytometry A. 2004 October; 61(2):189-95. Cell viability can also be evaluated via molecular viability analyses, e.g., a PCR-based approach, which can differentiate nucleic acids associated with viable cells from those associated with inactivated cells. See Cangelosi and Mescheke, Appl Environ Microbiol. 2014 October; 80(19): 5884-5891.

As used herein, “Shannon Diversity Index” refers to a diversity index that accounts for abundance and evenness of species present in a given community using the formula H=−Σᵢ₌₁^{R}pᵢ ln pᵢ, where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

As used herein, “adverse events (AEs)” refers to any dose that results in procedure- or microbiota-related signs or symptoms. As used herein, “serious adverse events (SAEs)” refers to any medical occurrence that at any dose: results in death or is life-threatening. As used herein “life-threatening” refers to an event in which the patient is at risk of death at the time of the event. Adverse events are graded according to a scale used by one of ordinary skill in the art (e.g., National Cancer Institute (NCI) Common Terminology Criteria for Adverse Events (CTCAE)).

Described herein are pharmaceutical compositions comprising bacteria, and methods of using the pharmaceutical composition for the treatment of ASD. Autism spectrum disorder (ASD) is a neurodevelopmental disorder that is characterized by impairments in social interaction and communication, restricted interests, and repetitive behavior. Individuals on the autism spectrum experience widely varying degrees and types of impairments, from mild to severe. Although early detection and interventions are encouraged to maximize the benefits and reduce the severity of the symptoms, individuals of any age can benefit from interventions and therapies that can reduce symptoms and increase skills and abilities. Appropriate subjects for the methods described herein include, without limitation, humans diagnosed as having or suspected of having autism spectrum disorder. In some cases, appropriate subjects for the methods provided herein are considered to be at increased risk (e.g., moderate or high risk) of developing ASD. In some cases, the subject has been diagnosed as having a condition meeting diagnostic criteria for ASD as set forth in the DSM-V. In other cases, the subject has a well-established DSM-IV diagnosis of autistic disorder, Asperger's disorder, or pervasive developmental disorder not otherwise specified (PDD-NOS).

Without being bound by theory, the microbiome function can be linked with ASD symptoms via several interrelated pathways including, for example, impaired gut barrier integrity, modulated mucosal immune tissue, and improperly stimulated vagus nerve. Further, without being bound by theory, the ASD microbiome can induce changes in mucosal immune cells that influence local and systemic inflammatory tone. Altered immunological activity in children with ASD is associated with altered microbiome composition and more pronounced in the subset of children with ASD that also experience GI symptoms. For example, mucosal immune cell population function is altered towards an inflammatory profile in children with ASD and GI symptoms. Peripheral Treg populations are decreased in all ASD children while an altered ratio of Treg to inflammatory Th17 cells is only found in children with ASD and GI symptoms. The lower Treg to inflammatory Th17 ratio is reminiscent of autoimmune disorders. Moreover, altered immune cell populations are found in the brains of individuals with ASD. In the cortex of ASD brains, pore-forming tight junction proteins are increased and barrier sealing TJ proteins are decreased indicating altered blood brain barrier function. Perivascular lymphocytes (CD4+, CD8+ T cells and B cells) around blood vessels are significantly increased in the cortex of individuals with ASD indicating increased inflammatory activity.

In an aspect, an ASD subject treated here exhibits reduced barrier-protective SCFA (e.g., butyrate) production and/or increased barrier-disruptive phenols (e.g., 4EPS, p-cresol). In another aspect, an ASD subject treated here exhibits impaired gut barrier integrity, which can lead to one or more of the following: increased translocation of bacterial products and GI metabolites, altered serum metabolites, local inflammation and GI distress, and LPS-induced activation of IFNγ, IL-6 and TNF leading to behavioral abnormalities.

In an aspect, an ASD subject treated here exhibits divergence in microbiome diversity and composition, and/or altered expression of MAMPs (eg LPS) signals local immune populations. In another aspect, an ASD subject treated here exhibits modulated mucosal immune tissue which can lead to one or more of the following: changes in mucosal and peripheral populations of Treg and Th populations toward a more inflammatory profile, induction of IL-17a and other cytokines, local inflammation and GI distress, and systemic/neuronal inflammation and behavioral abnormalities.

In an aspect, an ASD subject treated here exhibits microbial stimulation of enteric nervous system. In another aspect, an ASD subject treated here exhibits improperly stimulated vagus nerve which can lead to one or more of the following: vagus-dependent induction of paraventricular nucleus activity, endogenous production of oxytocin and other neuropeptides, modulation of behavior, possible modulation of enteric nervous system with impacts on GI-motility.

In aspects of the present disclosure, a pharmaceutical composition comprises a bacterial mixture comprising a preparation of uncultured fecal bacteria, for example non-selected fecal bacteria. In an aspect, a bacterial mixture comprises a single bacterial isolate or multiple bacterial isolates (e.g., in the form of a bacterial cocktail). In an aspect, a pharmaceutical composition comprises a bacterial mixture comprising (i) a preparation of uncultured fecal bacteria; and (ii) at least one bacterial isolate. Such a bacterial mixture can be referred to as a preparation of uncultured fecal bacteria enriched, supplemented or “spiked” with one or more bacterial isolates. By enriching or spiking a preparation of uncultured fecal bacteria derived from a stool sample (e.g., a fecal microbiota) of a healthy donor with one or more non-pathogenic bacterial isolates, a composition can be produced in which the amount of a particular bacterial strain or strains (i.e. the spiked-in bacterial isolate(s)) can be accounted for and precisely controlled. Without being bound by theory, this is advantageous, for example, where the at least one bacterial isolate spiked into the preparation of uncultured fecal bacteria is important for or involved in the treatment of a subject (e.g., having or susceptible to acquiring one or more symptoms of ASD), but insufficient on its own to generate an enhanced or optimal treatment response in the subject. Probiotics are relied on for their effect associated with the administration of a single bacterial isolate or a few bacterial isolates. Unlike probiotics, administration to an ASD subject of one or more bacterial isolates together with a preparation of uncultured fecal bacteria (i.e., derived from a healthy donor) provides the subject with the advantage of the administered bacterial isolate combined with multi-factorial benefits conferred by the additional fecal bacterial strains present in the preparation of uncultured microbes. These additional fecal bacterial strains may combine to, for example, provide for the necessary context or interactions (e.g. via one or more released factors) to enable the bacterial isolate to induce an optimal response in the subject, or may directly induce a response in the subject that combines and/or synergizes with a response induced by the bacterial isolate to treat the subject (e.g. the modulation of production of cytokine from a host cell). Accordingly, in certain aspects, a pharmaceutical composition comprising a mixture of one or more bacterial isolates and a preparation of uncultured fecal bacteria can be more effective in treating a subject (e.g., having or susceptible to acquiring one or more symptoms of ASD) than a composition comprising the bacterial isolate alone.

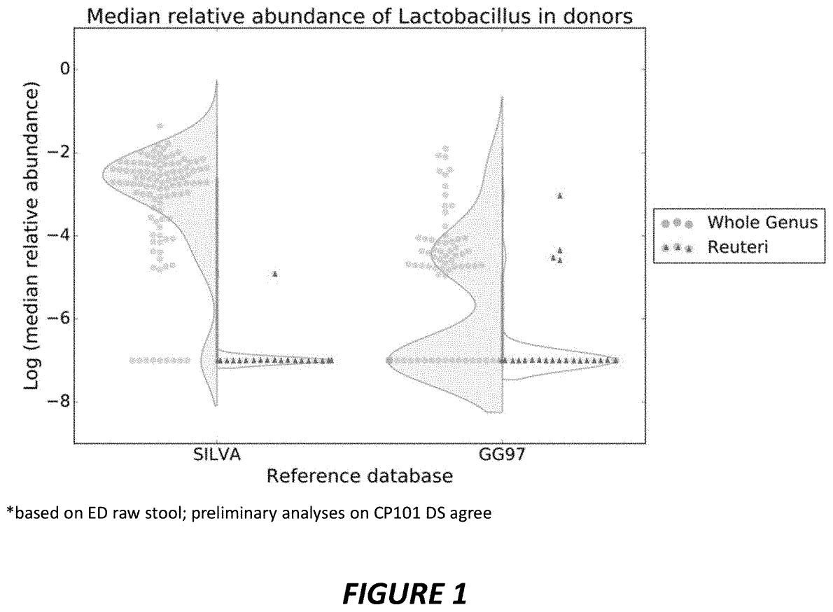

Enriching, supplementing, or spiking a preparation of uncultured fecal bacteria with one or more microbial isolates (to produce a “spiked” version of a preparation of uncultured fecal bacteria) possesses many advantages over a preparation of uncultured fecal bacteria without the enriched, supplemented or spiked microbial isolates. For example, first, without being bound by theory, one or more bacterial isolates added to the preparation of uncultured fecal bacteria may be unrepresented, or exist only at a low relative abundance, among the bacterial strains in the donor-derived stool used to manufacture a preparation of uncultured fecal bacteria. The addition of the bacterial isolate(s) to the preparation of uncultured fecal bacteria can therefore increase the relative abundance of one or more corresponding bacterial strains (i.e., originating from the one or more spiked-in bacterial isolates) in the gut of a patient administered the bacterial mixture, thereby increasing the likelihood that a desired bacterial strain will engraft in the gut of the patient. For example, a bacterial strain (e.g., L. reuteri) of relatively low abundance in a preparation of uncultured fecal bacteria could reside in a donor's intestinal mucosal layer or small intestine and thus generally not be present at a high level in a donor stool used to manufacture a preparation of uncultured fecal bacteria. Second, without being bound by theory, a spiked preparation of uncultured fecal bacteria can also mitigate heterogeneity in bacterial strain composition across donor stools (e.g., between stools collected from the same donor at different times or between stools of different donors). For example, certain bacterial strains are present or abundant in stool of some donors but absent or of low abundance in stool of others (e.g., L. reuteri, see FIG. 1). Such an issue can be addressed by supplementing a donor-derived preparation of uncultured fecal bacteria with one or more microbial isolates that show variable levels across donors. Third, without being bound by theory, a spiked version can also facilitate or augment redundancy of important strains or function. The presence of a strain in a donor-derived fecal extract drug product does not guarantee that the strain will engraft when administered to a patient. Engraftment success can depend on various factors such as the ecology of the patient gut microbiota, the abundance of the strain in the drug product, and the genetics of the microbial strain. If a microbial species is associated with a desired function in the gut of a patient, then the likelihood that the species/function will be introduced into the gut of a patient during treatment can be increased by co-administering multiple different strains of the species in the drug product. Thus, if a desired species is represented in the fecal microbial component of a drug product by a particular strain, introducing a different strain of the species in the spike-in component can increase the likelihood that one of the strains engrafts, to confer a desired function.

Enriching, supplementing, or spiking a preparation of uncultured fecal bacteria with one or more microbial isolates (e.g., to use a donor-derived fecal extract as a “backbone” to produce a spiked version) also possesses many advantages over using the same one or more microbial isolates without the preparation of uncultured fecal bacteria (e.g., either a single bacterial isolate or consortia of bacterial isolates). First, without being bound by theory, a spiked version enables a reduction in complexity (i.e., number of isolates) of a consortia of isolates administered to treat a patient, without compromising product quality, which improves manufacturing timelines and reduces costs. Second, without being bound by theory, a spiked composition can potentially improve engraftment of one or more bacterial strains in the gut of a recipient. Third, without being bound by theory, if the mechanism of action underlying the prevention or treatment of a disorder by gut bacteria is unknown, then the inclusion in a composition of an entire community of microbes from a healthy donor ensures that the microbes underlying the mechanism of prevention or treatment are represented.

In another aspect, a pharmaceutical composition comprising a mixture of one or more bacterial isolates and a preparation of uncultured fecal bacteria can be more effective in treating a subject (e.g., having or susceptible to acquiring one or more symptoms of ASD) than a composition comprising a preparation of uncultured fecal bacteria alone. For example, without being bound by theory, a bacterial isolate added to a preparation of uncultured fecal bacteria may possess an activity (i.e., effective for treating or preventing one or more symptoms of ASD) that is lacking in the preparation of uncultured fecal bacteria, for example because the preparation of uncultured fecal bacteria lacks a bacterial strain of the same taxonomic category as the bacterial isolate (or lacks a bacterial strain having a % genetic identity with the bacterial isolate above a threshold level), or because an abundance of a bacterial strain corresponding genetically to the bacterial isolate (e.g., in the same taxonomic category as the bacterial isolate) is below a threshold level in the preparation of uncultured fecal bacteria.

In an aspect, a pharmaceutical composition comprises a bacterial isolate and a preparation of uncultured fecal bacteria (e.g., prepared from a fecal microbiota of a healthy human donor) which lacks a bacterial strain of the same taxonomic category as the bacterial isolate. For example, the bacterial isolate can be of a phylum, class, order, family, genus or species that is not present in the preparation of uncultured fecal bacteria. In another aspect, a pharmaceutical composition comprises a bacterial isolate and a preparation of uncultured fecal bacteria which lacks a bacterial strain having 100% genetic identity with the bacterial isolate (e.g., as determined by a comparison of genetic identity between a 16S rRNA sequence of the bacterial isolate and 16S rRNA sequences of the bacterial strains of the preparation of uncultured fecal bacteria, or between whole genome sequences). In another aspect, a pharmaceutical composition comprises a bacterial isolate and a preparation of uncultured fecal bacteria which lacks a bacterial strain having greater than 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, or 99.9% genetic identity with the bacterial isolate (e.g., as determined by a comparison of genetic identity between a 16S rRNA sequence of the bacterial isolate and 16S rRNA sequences of the bacterial strains of the preparation of uncultured fecal bacteria, or between whole genome sequences).

In another aspect, a pharmaceutical composition comprises a bacterial isolate and a preparation of uncultured fecal bacteria (e.g., prepared from a fecal microbiota of a healthy human donor) which comprises one or more bacterial strains of the same taxonomic category as the bacterial isolate, but at an abundance or relative abundance below a threshold level. For example, the preparation of uncultured fecal bacteria can comprise one or more bacterial strains of the same phylum, class, order, family, genus or species as the bacterial isolate, but at an abundance or relative abundance that is below a threshold level. In various aspects, the one or more bacterial strains of the preparation of uncultured fecal bacteria of the same taxonomic category as the bacterial isolate can be below a threshold abundance of 10¹, 10², 10³, 10⁴, 10⁵, 10⁶, 10⁷, 10⁸, 10⁹, or 10¹⁰ CFUs per unit weight (e.g., grams) of the preparation of uncultured fecal bacteria. In various aspects, the one or more bacterial strains of the preparation of uncultured fecal bacteria of the same taxonomic category as the bacterial isolate can be below a threshold relative abundance of 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 15%, 16%, 18%, 20%, 22%, 24%, 25%, 26%, 28%, 30%, 35%, 40%, or 50% in a preparation of uncultured fecal bacteria.

In an aspect, a pharmaceutical composition comprises a bacterial mixture comprising a bacterial isolate and a preparation of uncultured fecal bacteria, such that a relative abundance of viable cells of the bacterial isolate in the bacterial mixture is less than a relative abundance of viable cells of the preparation of uncultured fecal bacteria (i.e., where the bacterial mixture comprises only one bacterial isolate, less than 50% of the viable cells of the bacterial mixture are cells of the bacterial isolate). In an aspect, a relative abundance of viable cells of the bacterial isolate in a bacterial mixture comprising the bacterial isolate and a preparation of uncultured fecal bacteria is less than 50%, less than 45%, less than 40%, less than 35%, less than 30%, less than 25%, less than 20%, less than 15%, less than 10%, less than 5%, less than 3%, or less than 1%.

In an aspect, a pharmaceutical composition comprises a bacterial mixture comprising a bacterial isolate and a preparation of uncultured fecal bacteria, such that a relative abundance of viable cells of the preparation of uncultured fecal bacteria in the bacterial mixture is less than a relative abundance of viable cells of the bacterial isolate (i.e., where the bacterial mixture comprises only one bacterial isolate, less than 50% of the viable cells of the bacterial mixture are cells of the preparation of uncultured fecal bacteria). In another aspect, a relative abundance of viable cells of the preparation of uncultured fecal bacteria in a bacterial mixture comprising the preparation of uncultured fecal bacteria and a bacteria isolate is less than 50%, less than 45%, less than 40%, less than 35%, less than 30%, less than 25%, less than 20%, less than 15%, less than 10%, less than 5%, less than 3%, or less than 1%.

In an aspect, a pharmaceutical composition comprises a bacterial mixture comprising a bacterial isolate and a preparation of uncultured fecal bacteria, such that a relative abundance of viable cells of the preparation of uncultured fecal bacteria is about equal to a relative abundance of viable cells of the bacterial isolate (i.e., where the bacterial mixture comprises only one bacterial isolate, about 50% of the viable cells of the bacterial mixture are cells of the bacterial isolate, and about 50% of the viable cells of the bacterial mixture are cells of the preparation of uncultured fecal bacteria).

In an aspect, a pharmaceutical composition comprises a bacterial mixture comprising a bacterial isolate and a preparation of uncultured fecal bacteria, such that a relative abundance of viable cells of the bacterial isolate is greater than the relative abundance of viable cells of any bacterial strain, any bacterial species, any bacterial genus, any bacterial family, any bacterial order, any bacterial class, or any bacterial phylum in the preparation of uncultured fecal bacteria.

In an aspect, a pharmaceutical composition comprises a bacterial mixture comprising at least one non-pathogenic bacterial isolate and/or a bacterial isolate having attenuated pathogenicity. A bacterial isolate can be isolated from any non-living (e.g., soil) or living source (e.g., animal), including a mammal such as a sheep, swine, bovine, primate, or human. If isolated from an animal, a bacterial isolate can be derived or isolated from any part of the animal, such as an organ, fluid or secretion, including an intestine, oral cavity, milk, saliva, or feces. In another aspect, a bacterial isolate is derived from a human. In another aspect, a bacterial isolate is derived from the fecal microbiota or intestinal microbiota of a human. In an aspect, a pharmaceutical composition administered herein comprises fecal bacteria. In another aspect, a pharmaceutical composition administered herein comprises one or more bacterial isolates extracted, isolated and/or cultured from a stool sample of a healthy human donor.

In some aspects, a bacterial isolate incorporated into a pharmaceutical composition described herein comprises live, vegetative cells. In some aspects, the bacterial isolate comprises bacteria capable of forming spores. In some aspects, the bacterial isolate comprises bacteria in the form of spores, e.g. viable spores. In some aspects, the bacterial isolate comprises bacteria in the form of live, vegetative cells and spores. In some aspects, a bacterial isolate is substantially free of live, vegetative cells. In some aspects, an entire bacterial cocktail is substantially free of live vegetative cells. In some aspects, a bacterial isolate is substantially free of spores. In some aspects, an entire bacterial cocktail is substantially free of spores.

In an aspect, a pharmaceutical composition can include a bacterial isolate (e.g., in combination with or spiked into a preparation of uncultured fecal bacteria) comprising, for example, a species of Lactobacillus, Bifidobacterium, Streptococcus, Clostridium, Collinsella, Dorea, Ruminococcus, Coprococcus, Prevotella, Veillonella, Bacteroides, Baccillus, or a combination thereof. In another aspect, a pharmaceutical composition can include a bacterial isolate comprising a species of Veillonellaceae, Firmicutes, Gammaproteobacteria, Bacteroidetes, or a combination thereof. In another aspect, a pharmaceutical composition can comprise a bacterial isolate comprising bacterial spores. In one aspect, fecal bacterial spores are Clostridium spores, Bacillus spores, or a combination thereof.

In an aspect, a pharmaceutical composition comprises a bacterial isolate comprising a species of Lactobacillus. In an aspect, a pharmaceutical composition comprises one or more, two or more, three or more, four or more, five or more, or six or more bacterial isolates each comprising a species of Lactobacillus. Non-limiting examples of a Lactobacillus species that can be incorporated into a pharmaceutical composition, alone or in combination, include L. acetotolerans, L. acidifarinae, L. acidipiscis, L. acidophilus, L. agilis, L. algidus, L. alimentarius, L. allii, L. alvei, L. alvi, L. amylolyticus, L. amylophilus, L. amylotrophicus, L. amylovorus, L. animalis, L. animata, L. antri, L. apinorum, L. apis, L. apodemi, L. aquaticus, L. aviarius, L. aviarius subsp. araffinosus, L. aviarius subsp. aviarius, L. backii, L. bambusae, L. bifermentans, L. bombi, L. bombicola, L. brantae, L. brevis, L. brevis subsp. coagulans, L. brevis subsp. gravesensis, L. brevisimilis, L. buchneri, L. cacaonum, L. camelliae, L. capillatus, L. casei, L. casei subsp. casei, L. chiayiensis, L. paracasei, L. paracasei subsp. paracasei, L. paracasei subsp. tolerans, L. zeae, L. catenefornis, L. caviae, L. cerevisiae, L. ceti, L. coleohominis, L. colini, L. collinoides, L. composti, L. concavus, L. coryniformis, L. coryniformis subsp. coryniformis, L. coryniformis subsp. torquens, L. crispatus, L. crustorum, L. curieae, L. curtus, L. curvatus, L. delbrueckii, L. delbrueckii subsp. bulgaricus, L. delbrueckii subsp. delbrueckii, L. delbrueckii subsp. indicus, L. delbrueckii subsp. jakobsenii, L. delbrueckii subsp. lactis, L. delbrueckii subsp. sunkii, L. dextrinicus, L. diolivorans, L. equi, L. equicursoris, L. equigenerosi, L. fabifermentans, L. faecis, L. faeni, L. farciminis, L. farraginis, L. fermentum, L. floricola, L. jlorum, L. formosensis, L. fornicalis, L. fructivorans, L. frumenti, L. fuchuensis, L. furfuricola, L. futsaii, L. gallinarum, L. gasseri, L. gastricus, L. ghanensis, L. gigeriorum, L. ginsenosidimutans, L. gorillae, L. graminis, L. guizhouensis, L. halophilus, L. hammesii, L. hamsteri, L. harbinensis, L. hayakitensis, L. heilongjiangensis, L. helsingborgensis, L. helveticus, L. helveticus subsp. jugurti, L. herbarum, L. heterohiochii, L. hilgardi, L. hokkaidonensis, L. hominis, L. homohiochii, L. hordei, L. iatae, L. iners, L. ingluviei, L. insectis, L. insicii, L. intermedius, L. intestinalis, L. iwatensis, L. ixorae, L. japonicus, L. jensenii, L. johnsonii, L. kalixensis, L. kefiranofacien, L. kefiranofaciens subsp. kefiranofaciens, L. kefiranofaciens subsp. kefirgranum, L. kefiri, L. kimbladii, L. kimchicus, L. kimchiensis, L. kisonensis, L. kitasatonis, L. koreensis, L. kosoi, L. kullabergensis, L. kunkeei, L. larvae, L. leichmannii, L. letivazi, L. lindneri, L. malefermentans, L. mall, L. manihotivorans, L. mellifer, L. mellis, L. melliventris, L. metriopterae, L. micheneri, L. mindensis, L. mixtipabuli, L. mobilis, L. modes tisalitolerans, L. mucosae, L. mudanjiangensis, L. murinus, L. musae, L. nagelii, L. namurensis, L. nantensis, L. nasuensis, L. nenjiangensis, L. nodensis, L. nuruki, L. odoratitofui, L. oeni, L. oligofermentans, L. oris, L. oryzae, L. otakiensis, L. ozensis, L. panis, L. panisapium, L. pantheris, L. parabrevis, L. parabuchneri, L. paracollinoides, L. parafarraginis, L. paragasseri, L. parakefiri, L. paralimentarius, L. paraplantarum, L. pasteurii, L. paucivorans, L. pentosiphilus, L. pentosus, L. perolens, L. plajomi, L. plantarum, L. plantarum subsp. argentoratensis, L. plantarum subsp. plantarum, L. pobuzihii, L. pontis, L. porci, L. porcinae, L. psittaci, L. quenuiae, L. raoultii, L. rapi, L. rennanquilb), L. rennini, L. reuteri, L. rhamnosus, L. rodentium, L. rogosae, L. rossiae, L. ruminis, L. saerimneri, L. sakei, L. sakei subsp. carnosus, L. sakei subsp. sakei, L. salivarius, L. sanfranciscensis, L. saniviri, L. satsumensis, L. secaliphilus, L. selangorensis, L. senioris, L. senmaizukei, L. sharpeae, L. shenzhenensis, L. sicerae, L. silage, L. silagincola, L. siliginis, L. similis, L. son ghuajiangensis, L. spicheri, L. sucicola, L. suebicus, L. sunkii, L. taiwanensis, L. terrae, L. thailandensis, L. timberlakei, L. timonensis, L. tucceti, L. ultunensis, L. uvarum, L. vaccinostercus, L. vaginalis, L. vermiforme, L. versmoldensis, L. vespulae, L. vini, L. wasatchensis, L. xiangfangensis, L. yonginensis, L. zymae, or a combination thereof.

In an aspect, a pharmaceutical composition comprises a bacterial isolate comprising Lactobacillus reuteri. Non-limiting and exemplary L. reuteri bacterial isolates that can be incorporated into a pharmaceutical composition described herein include ATCC PTA 6475, DSM 17938, ATCC 55730, ATCC PTA 5289, DSM 12246, RC-14® (CHR Hansen), or a combination thereof.

In an aspect, a pharmaceutical composition comprises a bacterial isolate comprising Lactobacillus plantarum. Non-limiting examples of an L. plantarum bacterial isolate that can be incorporated into a pharmaceutical composition described herein include WCFS1, ATCC BAA-793, ATCC BAA-2838, ATCC 10241, ATCC 10012, ATCC 8014, or a combination thereof. In another aspect, an L. plantarum bacterial isolate is Lactobacillus plantarum subsp. plantarum PSI 28. In another aspect, an L. plantarum bacterial isolate is DSMZ Accession No. DSM 28632 (See US20150306157A1).

In an aspect, a pharmaceutical composition comprises a bacterial isolate comprising L. rhamnosus. Non-limiting examples of an L. rhamnosus bacterial isolate that can be incorporated into a pharmaceutical composition described herein include ATCC 7469, ATCC 53103, ATCC 9595, 19070-2, or a combination thereof.

In an aspect, a pharmaceutical composition comprises a bacterial isolate comprising L. acidophilus. Non-limiting examples of an L. acidophilus bacterial isolate that can be incorporated into a pharmaceutical composition described herein include ATCC 4356, ATCC 53671, or a combination thereof.

In an aspect, a pharmaceutical composition comprises a bacterial isolate comprising L. delbrueckii subsp. bulgaricus. Non-limiting examples of L. delbrueckii subsp. bulgaricus bacterial isolates that can be incorporated into a pharmaceutical composition described herein include ATCC 11842, ATCC BAA-365, and a combination thereof.

In an aspect, a pharmaceutical composition comprises a bacterial isolate comprising L. paracasei. Non-limiting examples of an L. paracasei bacterial isolate that can be incorporated into a pharmaceutical composition described herein include ATCC 25302, ATCC 11578, ATCC 27216, ATCC 25598, or a combination thereof.

In an aspect, a pharmaceutical composition comprises a bacterial isolate comprising a species of Bifidobacterium. In an aspect, a pharmaceutical composition comprises a bacterial isolate comprising Bifidobacterium longum (e.g., UCD272, ATCC 15707, ATCC BAA-2753, or a combination thereof). In an aspect, a pharmaceutical composition comprises a bacterial isolate comprising the species Bifidobacterium longum, subsp. infantis (e.g., ATCC 15697). In an aspect, a pharmaceutical composition comprises a bacterial isolate comprising the species Bifidobacterium breve (e.g., ATCC 15700, ATCC 15698, ATCC 15701, or a combination thereof).

In an aspect, a pharmaceutical composition comprises a bacterial isolate comprising a species of Streptococcus. In an aspect, a pharmaceutical composition comprises a bacterial isolate comprising Streptococcus salivarius, subsp. thermophilus (e.g., ATCC 19258, ATCC BAA-491, ATCC 14485, or a combination thereof).

In an aspect, a pharmaceutical composition comprises a bacterial isolate comprising a species of Bacteroides. In an aspect, a pharmaceutical composition comprises a bacterial isolate comprising Bacteroides fragilis (e.g., ATCC 25285 or ATCC 23745).

In an aspect, a pharmaceutical composition comprises a bacterial mixture comprising two or more of the above bacterial isolates or bacterial strains. For example, in an aspect a pharmaceutical composition can comprise a bacterial isolate comprising L. reuteri (e.g., DSM 12246) in combination with a bacterial isolate comprising L. rhamnosus (e.g., 19070-2). In an aspect, a pharmaceutical composition can comprise two or more, three or more, four or more, five or more, six or more, seven or more, or eight bacterial isolates, each bacterial isolate comprising one of Streptococcus thermophilus, Bifidobacterium breve, Bifidobacterium longum, Bifidobacterium longum, subsp. infantis, Lactobacillus acidophilus, Lactobacillus plantarum, Lactobacillus paracasei, or Lactobacillus delbrueckii subsp. bulgaricus. In an aspect, a pharmaceutical composition can comprise multiple bacterial isolates in the form of a bacterial cocktail and/or probiotic. An example of a bacterial cocktail that can be included in the pharmaceutical composition is VSL #3® (Alfasigma®).

In an aspect, a bacterial isolate incorporated into a pharmaceutical composition described herein is a probiotic, or an ingredient in a probiotic. In an aspect, multiple bacterial isolates incorporated into a pharmaceutical composition described herein are probiotics, or ingredients in a probiotic. In an aspect, one or more bacterial isolates are in the form of a probiotic when incorporated into a pharmaceutical composition.

In aspects of the present disclosure, a pharmaceutical composition can comprise a bacterial mixture comprising multiple bacterial isolates (e.g. as a bacterial cocktail). In aspects of the present disclosure, the bacterial mixture can comprise at least two bacterial isolates, at least three bacterial isolates, at least four bacterial isolates, at least five bacterial isolates, at least six bacterial isolates, at least seven bacterial isolates, at least eight bacterial isolates, at least nine bacterial isolates, at least ten bacterial isolates, or a greater number of bacterial isolates, e.g., fifteen, twenty, twenty-five, thirty, or more bacterial isolates.

In various aspects, a pharmaceutical composition comprises one or more bacterial isolates capable of engrafting in a subject's GI tract following administration of the composition to the subject. Herein “engrafting” or “engraftment” refers to the stable presence over time of cells of a bacterial strain or bacterial isolate in the intestinal tract of a subject (e.g., after introducing the bacterial strain or isolate into the subject's intestinal tract by administering a composition described herein, for example, orally or rectally). Typically, engraftment of a bacterial isolate introduced into the intestine of a subject (e.g. by oral and/or rectal administration) is measured longitudinally, or over time, by comparing the abundance of the bacterial isolate in fecal samples of the subject before and after administration of the bacterial isolate to the subject. In an aspect, the bacterial isolate introduced into the intestine of the subject was absent prior to the administration. In another aspect, the bacterial isolate introduced into the intestine of the subject was present in the intestine prior to the administration, but is increased abundance following the administration. In certain aspects, engraftment is determined by identifying an increase in abundance of a bacterial strain administered to an intestine of the subject after at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14, days, at least 1 week, at least 2 weeks, at least 3 weeks, at least 4 weeks, at least 5 weeks, at least 6 weeks, at least 7 weeks, at least 8 weeks, at least 1 month, at least 2 months, at least 3 months, at least 4 months, at least 5 months, at least 6 months, or greater than 6 months following administration of the bacterial strain to the subject.

In aspects of the present disclosure, engraftment of a bacterial isolate in an intestine of a subject occurs when the bacterial isolate is administered to the subject at or above a threshold dose. In aspects of the present disclosure, engraftment of a bacterial isolate in an intestine of a subject does not occur, or occurs with relative inefficiency (e.g., across patients), when the bacterial isolate is administered to the subject below the threshold dose. For example, engraftment of a bacterial isolate into the intestine of a subject can occur when the bacterial isolate is administered to the subject (e.g., orally or rectally in a pharmaceutical composition described herein) at a dose of at least 10⁶ cells, at least 10⁷ cells, at least 10⁸ cells, at least 10⁹ cells, at least 10¹⁰ cells, at least 10¹¹ cells, or at least 10¹² cells.

In aspects of the present disclosure, engraftment of a bacterial isolate in an intestine of a subject occurs when the bacterial isolate is administered to the subject at or below a threshold dose. In an aspect, engraftment of a bacterial isolate in an intestine of a subject does not occur, or occurs with relative inefficiency (e.g., across patients), when the bacterial isolate is administered to the subject above the threshold dose. For example, engraftment of a bacterial isolate into the intestine of a subject can occur when the bacterial isolate is administered to the subject (e.g., orally or rectally in a pharmaceutical composition described herein) at a dose of not more than 10⁸ cells, not more than 10⁹ cells, not more than 10¹⁰ cells, not more than 10¹¹ cells, or not more than 10¹² cells.

In an aspect, a dose of one or more bacterial isolates to a patient in need thereof can depend on the engraftment threshold of the bacterial isolate.

In an aspect, a bacterial isolate in a pharmaceutical composition administered to a subject engrafts in the duodenum of the subject. In an aspect, a bacterial isolate in a pharmaceutical composition administered to a subject engrafts in the jejunum of the subject. In an aspect, a bacterial isolate in a pharmaceutical composition administered to a subject engrafts in the ileum of the subject. In an aspect, a bacterial isolate in a pharmaceutical composition administered to a subject engrafts in the colon of the subject.

In some aspects, a bacterial isolate is a non-pathogenic bacterial strain. In an aspect, a non-pathogenic bacterial strain comprises a genome that lacks genes, or expression thereof, which cause virulence and/or toxicity. For instance, in some aspects, a bacterial cocktail comprising one or more bacterial isolates is substantially free of organisms or entities (e.g., substantially free of pathogenic bacteria) which are capable of causing a disease or disorder in a subject administered the bacterial cocktail.

In an aspect, a bacterial isolate can be obtained from a laboratory stock or a bacterial cell bank of a bacterial strain originally obtained from a stool sample of a healthy human donor. For example, a fecal microbiota (e.g., purified from a stool sample using methods described herein) can be used as the source of a bacterial isolate incorporated into a pharmaceutical composition described herein. In certain aspects, all or a portion of a fecal microbiota of a stool sample is cultured on a solid media substrate and one or more bacterial isolates are identified as single colonies. In other aspects, all or a portion of a fecal microbiota can be inoculated into liquid culture to produce a mixed bacterial culture that is then serially diluted to produce a culture containing a single cell of a bacterial isolate. In an aspect, an identified bacterial isolate can then be cultured (e.g., in solid or liquid media) using known techniques and expanded. Methods for isolating, purifying, and/or culturing bacterial strains are described in Sadowsky et al., WO 2012/122478 and described in Borody et al., WO 2012/016287, each of which is incorporated herein by reference.

In an aspect, a bacterial mixture described herein modulates cytokine production or release by a eukaryotic cell (e.g. a cell of a mammal administered the bacterial mixture to treat ASD). Herein “eukaryotic cell” refers to both a cell (e.g. intestinal cell) positioned ‘in situ’ within a body of a subject administered a composition described herein, as well as a cell grown or growing “ex vivo” outside of an organism, for example, in culture medium.

In an aspect, a bacterial mixture described herein, once administered to a subject, can modulate production of a cytokine in a cell of the subject (referred to herein as a “host cell”). In embodiments, bacteria in the bacterial mixture modulate production and/or secretion of a cytokine from a host cell of the subject, wherein the cytokine tends to exert anti-inflammatory effects on a tissue of the subject (e.g., intestinal tissue). Examples of such anti-inflammatory cytokines that can be produced and/or secreted from a host cell in response to the presence of bacteria (e.g. a bacterial isolate) administered in a composition described herein include IL-10, IL-13, IL-4, IL-5, TGF-β, GM-CSF and a combination thereof. In other embodiments, a bacterial mixture administered in a composition described herein inhibits production and/or secretion of a cytokine from a host cell of a subject, wherein the cytokine tends to exert pro-inflammatory effects on a tissue of the subject (e.g., intestinal tissue). Examples of such pro-inflammatory cytokines include IFNγ, IL-12p70, IL-1 (e.g., IL-1α, IL-1β), IL-6, IL-8, IL-12, IL-17, IL-18, IL-23, MCP1, MIP1α, MIP1β, TNFα, TNF-γ, and a combination thereof. By providing a composition containing a bacterial mixture that can induce a host cell to produce or secrete an anti-inflammatory cytokine and/or inhibit production and/or secretion by the host cell of a pro-inflammatory cytokine, the compositions described herein can treat, alleviate, inhibit, and/or prevent inflammation associated with autism. Herein a bacterial mixture or bacterial isolate capable of modulating cytokine production and/or secretion by a host cell is referred to as “immunomodulatory”.

In embodiments, a bacterial mixture can directly and/or indirectly modulate production and/or release of a cytokine from a cell of a subject administered a pharmaceutical composition. In one embodiment immunomodulatory bacteria (e.g. a bacterial isolate) can act directly on a host cell of a subject via, for example, microbe-associated molecular patterns (MAMPS) secreted by bacteria in the bacterial mixture or displayed on the surface of the bacteria. Such MAMPS play a major role in host immune responses to particular bacterial species. MAMPS are sensed by pattern recognition receptors (PRRs) expressed on most host cell types that are in contact with bacteria. Examples of MAMPS of bacteria in a bacterial mixture described herein include unmethylated 2-deoxyribo(cytidine-phosphate-guanine) (CpG) dinucleotides, bacterial peptidogylcans, bacterial lipopolysaccharides (LPS, which interacts with co-receptors MD-2, CD14, and LPB to facilitate high affinity binding to TLR-4 and subsequent host cell activation), bacterial lipoproteins (LPs), lipoteichoic acid, flagellin, membrane vesicles, and exopolysaccharides. Examples of PRRs expressed by host cells in the gut and resident intestinal immune cells that can mediate modulation of cytokine production via interaction with MAMPS include Toll-like receptors (TLRs), nucleotide-binding oligomerization domains (Nods), NOD like receptors and C-type lectins. The interaction of intestinal cell PRRs and microbial ligands trigger signaling pathways associated with the innate and adaptive immune systems that are required to maintain immune tolerance and intestinal health.

In another embodiment, immunomodulatory bacteria can act indirectly on a cell (e.g., immune cell) of a subject administered a pharmaceutical composition by, for example, secreting a metabolite that modulates the activity of the host cell of the subject, for example, by inducing the cell to express a cytokine.

Examples of host cells whose production and/or release of cytokines can be modulated by a bacterial mixture described herein include an intestinal cell, an epithelial cell, an intestinal mucosal cell, an intestinal epithelial cell, an intestinal lamina propria cell, an endothelial cell, fibroblast, a stromal cell, a macrophage, a B lymphocyte, a T lymphocyte, a mast cell, and a peripheral blood mononuclear cell (PBMC).

In another embodiment, a bacterial mixture described herein can modulate cytokine production and/or release (e.g., increase cytokine production) by a eukaryotic cell (e.g., PBMC) situated or growing in culture medium, when the bacterial isolate is co-cultured with the eukaryotic cell.

In an aspect, a bacterial mixture comprising a combination of one or more bacterial isolates (e.g. a Lactobacillus isolate) and a preparation of uncultured fecal bacteria is administered to a subject to modulate cytokine production (e.g. by increasing a level of an anti-inflammatory cytokine or decreasing the level of a pro-inflammatory cytokine). In embodiments, administration of a bacterial mixture comprising a combination of a bacterial isolate with a preparation of uncultured fecal bacteria modulates the level of cytokine produced by a eukaryotic cell to a greater extent than the modulation produced by administration of either the uncultured fecal bacteria or the bacterial isolate alone.

In certain embodiments, a bacterial isolate described herein can induce an anti-inflammatory cytokine profile. In an embodiment, a bacterial isolate exhibits an anti-inflammatory cytokine profile when it produces a level of IL-10 that is increased relative to that of a control strain. In an embodiment, a bacterial isolate exhibits an anti-inflammatory cytokine profile when it produces a level of IL-12 that is decreased relative to that of a control strain. In an embodiment, a bacterial isolate exhibits an anti-inflammatory cytokine profile when it produces a level of GM-CSF that is increased relative to that of a control strain. In an embodiment, a bacterial isolate exhibits an anti-inflammatory cytokine profile when it produces a level of IFN-gamma that is decreased relative to that of a control strain. In an embodiment, a bacterial isolate exhibits an anti-inflammatory cytokine profile when it produces a level of TNF-alpha that is decreased relative to that of a control strain. In an embodiment, a bacterial isolate exhibits an anti-inflammatory cytokine profile when it produces a level of IL-23 that is decreased relative to that of a control strain. In an embodiment, a bacterial isolate exhibits an anti-inflammatory cytokine profile when it produces a level of IL-12 that is decreased relative to that of a control strain. In an embodiment, a bacterial isolate exhibits an anti-inflammatory cytokine profile when it produces a ratio of IL-10:IL-12 that is increased relative to that of a control strain. In an embodiment, a bacterial isolate exhibits an anti-inflammatory cytokine profile when it produces a ratio of IL-10:TNF-alpha that is increased relative to that of a control strain.

In an aspect, a pharmaceutical composition comprises a bacterial mixture comprising a preparation of uncultured fecal bacteria, for example non-selected fecal bacteria and/or a substantially complete fecal microbiota of a stool or portion thereof (e.g., from a healthy human donor). Herein the term “substantially complete fecal microbiota” refers to a preparation of uncultured fecal bacteria that comprises viable bacterial cells from all or substantially all of the bacterial taxa represented among the viable bacterial cells in the stool from which the fecal microbiota was extracted. In an aspect, the relative abundance of viable bacterial cells from at least two of the taxa in the substantially complete fecal microbiota is proportional to the relative abundance of the viable cells from those taxa in the stool from which the fecal microbiota was extracted. In an aspect, the bacterial mixture further comprises one or more bacterial isolates. In an aspect, the bacterial mixture does not comprise a bacterial isolate.

In one aspect, a preparation of uncultured fecal bacteria comprises a donor's entire or substantially complete fecal microbiota from a stool sample. In one aspect, a preparation of uncultured fecal bacteria comprises a non-selective fecal microbiota. In another aspect, a preparation of uncultured fecal bacteria comprises an isolated or purified population or community of live non-pathogenic fecal bacteria. In a further aspect, a preparation of uncultured fecal bacteria comprises anon-selective and substantially complete fecal microbiota preparation from a single donor. In another aspect, a pharmaceutical composition used herein comprises a mixture of live, non-pathogenic, bacterial isolates and live, non-pathogenic, purified or extracted, preparation of uncultured fecal bacteria.

In an aspect, the manufacture of a preparation of uncultured fecal bacteria involves a treatment selected from the group consisting of ethanol treatment, detergent treatment, heat treatment, irradiation, and sonication. In another aspect, the manufacture of a preparation of uncultured fecal bacteria involves no treatment selected from the group consisting of ethanol treatment, detergent treatment, heat treatment, irradiation, and sonication. In one aspect, the manufacture of a preparation of uncultured fecal bacteria involves a separation step selected from the group consisting of density gradients, filtration (e.g., sieves, nylon mesh), and chromatography. In another aspect, the manufacture of a preparation of uncultured fecal bacteria involves no separation step selected from the group consisting of density gradients, filtration (e.g., sieves, nylon mesh), and chromatography. In another aspect, a preparation of uncultured fecal bacteria comprises an entire or substantially entire fecal microbiota from a stool sample of a subject. In another aspect, a pharmaceutical composition administered herein comprises a fecal microbiota substantially free of donor eukaryotic cells.

In an aspect, a pharmaceutical composition provided or administered herein comprises a preparation of uncultured fecal bacteria comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a pharmaceutical composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a pharmaceutical composition comprises a preparation of flora in proportional content that resembles a normal healthy human fecal flora.

In a further aspect, a pharmaceutical composition comprises fecal bacteria from at least 1, 2, 3, 4, 5, 6, 7, 8, 9, or 10 different families. In another aspect, a pharmaceutical composition comprises fecal bacteria from at least 11, 12, 13, 14, 15, 16, 17, 18, 19, or 20 different families. In yet another aspect, a pharmaceutical composition comprises fecal bacteria from at least 21, 22, 23, 24, 25, 26, 27, 28, 29, or 30 different families. In a further aspect, a pharmaceutical composition comprises fecal bacteria from at least 31, 32, 33, 34, 35, 36, 37, 38, 39, or 40 different families. In another aspect, a pharmaceutical composition comprises fecal bacteria from at least 41, 42, 43, 44, 45, 46, 47, 48, 49, or 50 different families. In another aspect, a pharmaceutical composition comprises fecal bacteria from between 1 and 10, between 10 and 20, between 20 and 30, between 30 and 40, between 40 and 50 different families. In an aspect, a pharmaceutical composition provided or administered herein comprises a preparation of uncultured fecal bacteria comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a pharmaceutical composition provided or administered herein comprises an uncultured fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a pharmaceutical composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.33 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.002 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one aspect, “biological material” refers to the living material, e.g., the microbes, eukaryotic cells, and viruses, which are present in the colon of a normal healthy human. In an aspect, a pharmaceutical composition provided or administered herein comprises an extract of human stool, wherein the composition is substantially odorless. In an aspect, a pharmaceutical composition provided or administered herein comprises fecal material or a fecal floral preparation in a lyophilized, crude, semi-purified or purified formulation.

In an aspect, a preparation of uncultured fecal bacteria included in a pharmaceutical composition comprises highly refined or purified fecal flora, e.g., substantially free of non-floral fecal material. In an aspect, an uncultured fecal microbiota (comprising a preparation of uncultured fecal bacteria) harvested from a donor can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a preparation of uncultured fecal bacteria in a pharmaceutical composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a preparation of uncultured fecal bacteria included in a pharmaceutical composition comprises a donor's substantially entire or non-selected fecal microbiota. In another aspect, the fecal microbiota in a pharmaceutical composition comprises no antibiotic resistant population. In another aspect, a pharmaceutical composition comprises an uncultured fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, a preparation of uncultured fecal bacteria included in a pharmaceutical composition is derived from a disease-screened stool sample of a human donor. In an aspect, a stool sample does not include an antibiotic resistant population. For example, a composition can comprise a preparation of viable flora which can in proportional content, resemble normal healthy human fecal flora which does not include antibiotic resistant populations.

In one aspect, a preparation of uncultured fecal bacteria described and used herein comprises one or more, two or more, three or more, four or more, or five or more live fecal microorganisms selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbiota preparation comprises one or more, two or more, three or more, four or more, or five or more live fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridliformis ssp. clostridliformis, Bacteroides coagulans, Prevotella oxalis, Prevotella ruminicola, Odoribacter splanchnicus, and Desuifomonas pigra.

In one aspect, a fecal microbiota preparation described and used here lacks or is substantially devoid of one or more, two or more, three or more, four or more, or five or more live fecal microorganisms are selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbiota preparation lacks or is substantially devoid of one or more, two or more, three or more, four or more, or five or live more fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oxalis, Prevotella ruminicola, Odoribacter splanchnicus, and Desuifomonas pigra.

In an aspect, a preparation of uncultured fecal bacteria for incorporation into a pharmaceutical composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosurn, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum. In an aspect, a pharmaceutical composition comprises one or more, two or more, three or more, or four or more non-pathogenic Bacteroides species selected from the group of Bacteroides coprocola, Bacteroides plebeius, Bacteroides massiliensis, Bacteroides vulgatus, Bacteroides helcogenes, Bacteroides pyogenes, Bacteroides tectus, Bacteroides umformis, Bacteroides stercoris, Bacteroides eggerthii, Bacteroides finegoldii, Bacteroides thetaiotaomicron, Bacteroides ovatus, Bacteroides acidifaciens, Bacteroides caccae, Bacteroides nordii, Bacteroides salyersiae, Bacteroides fragilis, Bacteroides intestinalis, Bacteroides coprosuis, Bacteroides distasonis, Bacteroides goldsteinii, Bacteroides merdae, Bacteroides forsythus, Bacteroides splanchnicus, Bacteroides capillosus, Bacteroides cellulosolvens, and Bacteroides ureolyticus.

In an aspect, a pharmaceutical composition comprises viable non-pathogenic Clostridium and a plurality of viable non-pathogenic microorganisms from one or more genera selected from the group consisting of Collinsella, Coprococcus, Dorea, Eubacterium, and Ruminococcus. In another aspect, a pharmaceutical composition comprises a plurality of viable non-pathogenic microorganisms from one or more genera selected from the group consisting of Clostridium, Collinsella, Coprococcus, Dorea, Eubacterium, and Ruminococcus.

In an aspect, a pharmaceutical composition comprises two or more genera selected from the group consisting of Collinsella, Coprococcus, Dorea, Eubacterium, and Ruminococcus. In another aspect, a pharmaceutical composition comprises two or more genera selected from the group consisting of Coprococcus, Dorea, Eubacterium, and Ruminococcus. In a further aspect, a pharmaceutical composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In an aspect, a preparation of uncultured fecal bacteria described herein comprises viable cells from 100% of the viable bacterial taxa represented in the stool from which the fecal bacteria were derived. In an aspect, a preparation of uncultured fecal bacteria described herein comprises viable cells from at least 99% of the viable bacterial taxa represented in the stool from which the fecal bacteria were derived. In an aspect, a preparation of uncultured fecal bacteria described herein comprises viable cells from at least 98% of the viable bacterial taxa represented in the stool from which the fecal bacteria were derived. In an aspect, a preparation of uncultured fecal bacteria described herein comprises viable cells from at least 97% of the viable bacterial taxa represented in the stool from which the fecal bacteria were derived. In an aspect, a preparation of uncultured fecal bacteria described herein comprises viable cells from 96% of the viable bacterial taxa represented in the stool from which the fecal bacteria were derived. In an aspect, a preparation of uncultured fecal bacteria described herein comprises viable cells from at least 95, 94, 93, 92, 91, 90, 89, 88, 87, 85, 84, 83, 82, 81, 80, 75, 70, 65, 60, 55, 50, 45, or 40% of the viable bacterial taxa represented in the stool from which the fecal bacteria were derived.

In an aspect, a pharmaceutical composition disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, excreted or otherwise liquid components or a microbiota, e.g., biologically active molecules (BAMs), which can be antibiotics or anti-inflammatories, are preserved, retained or reconstituted in a flora extract.

In one aspect, an exemplary pharmaceutical composition comprising a fecal filtrate comprises starting material from a donor from a defined donor pool, where this donor contributes a stool that is homogenized and centrifuged, then filtered with very high-level filtration using e.g., either metal sieving or Millipore filters, or equivalent, to ultimately permit only cells of bacterial origin to remain, e.g., often less than about 5 micrometers diameter. After the initial centrifugation, the solid material can be separated from the liquid, and the solid is then filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation can in some aspects be filtered progressively in a filtering, e.g., a Millipore filtering or equivalent systems, to produce a liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a pharmaceutical composition comprises reconstituted fecal flora consisting essentially of a combination of a purified fecal microbiota (comprising a preparation of uncultured fecal bacteria) and a non-cellular fecal filtrate. In another aspect, a pharmaceutical composition comprises a purified fecal microbiota (comprising a preparation of uncultured fecal bacteria) supplemented with one or more non-cellular non-particulate fecal components. In one aspect, a pharmaceutical composition comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a pharmaceutical composition comprises both a preparation of uncultured fecal bacteria, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a pharmaceutical composition comprising a preparation of uncultured fecal bacteria is used concurrently with a fecal non-cellular filtrate-based pharmaceutical composition. In another aspect, a patient is treated with a first fecal non-cellular filtrate-based pharmaceutical composition before being given a second pharmaceutical composition comprising a preparation of uncultured fecal bacteria, or vice versa. In a further aspect, a treatment method comprises three steps: first, antibiotic pretreatment to non-selectively remove infectious pathogen(s); second, a fecal non-cellular filtrate-based treatment step to further suppress selected infectious pathogen(s); and third, treatment with a pharmaceutical composition comprising a preparation of uncultured fecal bacteria to re-establish a functional intestinal microbiome.

In an aspect, a composition comprising a bacterial mixture comprising a preparation of uncultured fecal bacteria that is administered to a subject (e.g., an ASD patient) effects a cure, reduction of the symptoms, or a percentage reduction of symptoms based on replacement of bacterial cells endogenous to the intestinal flora of the subject with bacterial cells from the administered bacterial mixture. The change of flora can be as “near-complete” as possible. Typically, the change in enteric flora comprises introduction of an array of flora derived from the stool of a healthy human donor into the gastro-intestinal system of the subject, which can substantially or completely displace pathogenic enteric flora in a patient requiring such treatment (e.g., an ASD patient).

The pharmaceutical compositions described here can comprise microbes, e.g. bacteria, derived from a stool sample of a donor, e.g. a healthy human donor. In an aspect, a composition incorporates a preparation of uncultured fecal bacteria derived from all or a portion of a fecal microbiota of a stool sample of a healthy human donor. For example, a composition can incorporate a substantially complete fecal microbiota of a stool sample of a healthy human donor. In an aspect, a composition incorporates a bacterial isolate of a fecal microbiota, wherein the bacterial isolate has been purified and/or cultured from all or a portion of the fecal microbiota of a stool sample from a healthy human donor. The harvesting, extraction and/or purification of a fecal microbiota from a stool sample can thus be performed to prepare a composition comprising at least one of a preparation of uncultured fecal bacteria or a bacterial isolate.

In one aspect, an exemplary fecal microbiota for use in preparing a composition described herein (e.g., comprising a bacterial mixture comprising one or more of a preparation of uncultured fecal bacteria and at least one bacterial isolate) comprises starting material from a human donor. In another aspect, an exemplary fecal microbiota comprises material from one or more healthy human donors. In yet another aspect, an exemplary fecal microbiota comprises starting material from a pool of known, defined donors. In another aspect, a donor is an adult male. In a further aspect, a donor is an adult female. In yet another aspect, a donor is an adolescent male. In another aspect, a donor is an adolescent female. In another aspect, a donor is a female toddler. In another aspect, a donor is a male toddler. In another aspect, a donor is healthy. In one aspect, a human donor is a child below about 18, 15, 12, 10, 8, 6, 4, 3, 2, or 1-year-old. In another aspect, a human donor is an elderly individual. In a further aspect, a human donor is an individual above about 30, 35, 40, 45, 50, 55, 60, 65, 70, 75, 80, 85, 90, or 95 years old. In another aspect, a donor is between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a donor is a young old individual (65-74 years). In one aspect, a donor is a middle old individual (75-84 years). In one aspect, a donor is an old individual (>85 years). In yet another aspect, a donor is a carefully screened, healthy, neurotypical human.

In an aspect, a fecal donor is prescreened for its fecal microbiome profile. In another aspect, a fecal donor is selected for the presence of one or more fecal bacterial class, family, genus, or species in the donor's stool. In another aspect, a fecal donor is selected for the presence of one or more fecal bacterial class, family, genus, species or strain in the donor's stool at a level above a threshold abundance. In an aspect, a fecal donor can be selected on the basis of the presence or threshold abundance of one or more bacterial genera in the stool of the donor selected from the group consisting of Lactobacillus, Bifidobacterium, Streptococcus, Prevotella, Desulfovibrio, and a combination thereof. In an aspect, a fecal donor can be selected on the basis of the presence or threshold abundance of one or more bacterial genera in the stool of the donor selected from the group consisting of Clostridium, Bacteroides, Eggerthella, Bifidobacterium, Prevotella, and Desulfovibrio and a combination thereof. In an aspect, a fecal donor can be selected on the basis of the presence or threshold abundance of one or more bacterial taxa in the stool of the donor selected from the group consisting of Prevotella, Coprococcus, Prevotellaceae, and Veillonellaceae, and a combination thereof. In an aspect, a fecal donor can be selected on the basis of the presence or threshold abundance of one or more bacterial genera selected from the group consisting of Lactobacillus, Bifidobacterium, Streptococcus, and a combination thereof. In an aspect, a fecal donor can be selected on the basis of the presence or threshold abundance in the stool of the donor of the genus Lactobacillus. In an aspect, the fecal donor can be selected on the basis of the presence or threshold abundance of Lactobacillus reuteri in the stool of the donor.

In an aspect, a stool sample can be selected as a source of a preparation of uncultured fecal bacteria for incorporation into a pharmaceutical composition on the basis of the presence or threshold abundance of one or more bacterial class, family, genus, species or strain in the stool sample. In an aspect, the stool sample can be selected on the basis of the presence or threshold abundance of a member of a bacterial genus selected from the group consisting of Lactobacillus, Bifidobacterium, Streptococcus, and a combination thereof. In an aspect, the stool sample can be selected on the basis of the presence or a threshold abundance of Lactobacillus reuteri in the stool sample.

A preparation of uncultured fecal bacteria extracted from the stool of a donor selected on the basis of the presence or abundance of one or more bacterial genera, species or strains (e.g. the presence or threshold abundance of L. reuteri) can be directly incorporated into a pharmaceutical composition described herein, without adding any bacterial isolate to the preparation, or alternatively can be spiked with a bacterial isolate of the same genera, species or strain as that which was the basis of selection.

In an aspect, a fecal donor has a higher relative fecal abundance of a bacterial genus, species or strain by ingesting a probiotic and/or prebiotic that promotes the proliferation or presence of the bacterial genus, species or strain in the donor's gut, compared to a relative fecal abundance of the bacterial genus, species or strain in the absence of ingesting the probiotic and/or prebiotic.

In another aspect, prior to making a fecal donation, a donor receives or ingests certain prebiotics such as oligofructose, inulin, barley prebiotics, or another dietary fiber. In another aspect, prior to making a fecal donation, a donor receives or ingests a growth stimulant for selected fecal bacteria. In another aspect, prior to making a fecal donation, a donor receives or ingests one or more of apple pectin, N-acetyl glucosamine, cysteine, glutathione, riboflavin, and flavin.

In an aspect, a carefully screened donor undergoes a complete medical history and physical exam. Donors are excluded if they have a risk of infectious agents. Additional exclusion criteria comprise the following:
1. Known viral infection with Hepatitis B, C or HIV
2. Known exposure to HIV or viral hepatitis at any time
3. High risk behaviors including sex for drugs or money, men who have sex with men, more than one sexual partner in the preceding 12 months, any past use of intravenous drugs or intranasal cocaine, history of incarceration.
4. Tattoo or body piercing within 12 months.
5. Travel to areas of the world where risk of traveler's diarrhea is higher than the US.
6. Current communicable disease, e.g., upper respiratory viral infection.
7. History of irritable bowel syndrome. Specific symptoms can include frequent abdominal cramps, excessive gas, bloating, abdominal distension, fecal urgency, diarrhea, constipation.
8. History of inflammatory bowel disease such as Crohn's disease, ulcerative colitis, microscopic colitis.
9. Chronic diarrhea.
10. Chronic constipation or use of laxatives.
11. History of gastrointestinal malignancy or known colon polyposis.
12. History of any abdominal surgery, e.g., gastric bypass, intestinal resection, appendectomy, cholecystectomy, etc.
13. Use of Probiotics or any other over the counter aids used by the potential donor for purpose of regulating digestion. Yogurt and kefir products are allowed if taken merely as food rather than nutritional supplements.
14. Antibiotics for any indication within the preceding 6 months.
15. Any prescribed immunosuppressive or anti-neoplastic medications.
16. Metabolic Syndrome, established or emerging. Criteria used for definition here are stricter than any established criteria. These include history of increased blood pressure, history of diabetes or glucose intolerance.
17. Known systemic autoimmunity, e.g., connective tissue disease, multiple sclerosis.
18. Known atopic diseases including asthma or eczema.
19. Chronic pain syndromes including fibromyalgia, chronic fatigue syndrome.
20. Ongoing (even if intermittent) use of any prescribed medications, including inhalers or topical creams and ointments.
21. Neurologic, neurodevelopmental, and neurodegenerative disorders including autism, Parkinson's disease.
22. General. Body mass index>26 kg/m2, central obesity defined by waste:hip ratio >0.85 (male) and >0.80 (female).
23. Blood pressure >135 mmHg systolic and >85 mmHg diastolic.
24. Skin—presence of a rash, tattoos or body piercing placed within a year, or jaundice
25. Enlarged lymph nodes.
26. Wheezing on auscultation.
27. Hepatomegaly or stigmata of liver disease.
28. Swollen or tender joints. Muscle weakness.
29. Abnormal neurologic examination.
30. Positive stool Clostridium difficile toxin B tested by PCR.
31. Positive stool cultures for any of the routine pathogens including Salmonella, Shigella, Yersinia, Campylobacter, E. coli 0157:H7.
32. Abnormal ova and parasites examination.
33. Positive Giardia, Cryptosporidium, or Helicobacter pylori antigens.
34. Positive screening for any viral illnesses, including HIV 1 and 2, Viral Hepatitis A IgM, Hepatitis surface antigen and core Ab.
35. Abnormal RPR (screen for syphilis).
36. Any abnormal liver function tests including alkaline phosphatase, aspartate aminotransaminase, alanine aminotransferase.
37. Raised serum triglycerides >150 mg/Dl
38. HDL cholesterol <40 mg/dL (males) and <50 mg/dL (females)
39. High sensitivity CRP >2.4 mg/L
40. Raised fasting plasma glucose (>100 mg/dL)

In one aspect, provided herein is a process for collecting and processing a stool sample to give rise to a preparation of uncultured fecal bacteria and/or one or more bacterial isolates.

The process can comprise first collecting a stool sample from one or more healthy (e.g., screened) donor(s). In one aspect, a fresh stool is transported via a stool collection device, which can provide or comprises a suitably oxygen free (or substantially oxygen free) appropriate container. In one aspect, the container can be made oxygen free by e.g., incorporating into the container a built in or clipped-on oxygen-scavenging mechanism, e.g., oxygen scavenging pellets as described e.g., in U.S. Pat. No. 7,541,091. In another aspect, the container itself is made of an oxygen scavenging material, e.g., oxygen scavenging iron, e.g., as described by O2BLOCK™, or equivalents, which uses a purified and modified layered clay as a performance-enhancing carrier of oxygen-scavenging iron; the active iron is dispersed directly in the polymer. In one aspect, oxygen-scavenging polymers are used to make the container itself or to coat the container, or as pellets to be added; e.g., as described in U.S. Pat. App. Pub. 20110045222 (hereby incorporated herein by reference in its entirety), describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one aspect, oxygen-scavenging polymers are used to make the container itself or to coat the container, or as pellets to be added; e.g., as described in U.S. Pat. App. Pub. 20110008554 (hereby incorporated herein by reference in its entirety), describing compositions comprising a polyester, a copolyester ether and an oxidation catalyst, wherein the copolyester ether comprises a polyether segment comprising poly(tetramethylene-co-alkylene ether). In one aspect, oxygen-scavenging polymers are used to make the container itself or to coat the container, or as pellets to be added; e.g., as described in U.S. Pat. App. Pub. 201000255231 (hereby incorporated herein by reference in its entirety), describing a dispersed iron/salt particle in a polymer matrix, and an oxygen scavenging film with oxygen scavenging particulates.

Alternatively, in addition to or in place of the oxygen-scavenging mechanism, the air in the container can be replaced (completely or substantially) with nitrogen and/or other inert non-reactive gas or gases. In one aspect, the container simulates (creates) partially, substantially or completely an anaerobic environment.

In one aspect, the stool (e.g., fecal sample) is held in an aesthetically acceptable container that will not leak nor smell yet maintain an anaerobic environment. In one aspect, the container is sterile before receiving the fecal flora.

In one aspect, a stool sample provided herein is maintained at room temperature during most or all of its transportation and/or storage at e.g., a “stool bank”. For example, once delivered to a “processing stool bank” it is stored at ambient temperature, e.g., room temperature. In one aspect, stabilizing agents, such as glycerol, are added to the harvested and/or stored material.

In one aspect, the stool is tested for various pathogens, as noted above. In one aspect, once cleared of infective agents, a stool sample is homogenized and filtered to remove large particles of matter. In one aspect, the stool is subdivided into desired volumes, e.g., which can be between 5 cc and 3 or more liters. For example, in one aspect, a container comprises a 50 gram (g) stool, which can be held in an appropriate oxygen resistant plastic, e.g., a metallized polyethylene terephthalate polyester film, or a metallized MYLAR™.

In one aspect, the stool is subject to homogenization by for example, mixing, agitating, stirring or shaking. In certain aspects, a stool sample is diluted with a homogenization buffer prior to homogenization. A homogenization buffer can, for example, contain a cryoprotectant (e.g., trehalose), an antioxidant or reducing agent (e.g., cysteine), and a buffer (e.g., 0.25×PBS at pH 7.4).

In one aspect, to separate the non-bacterial components from the fecal microbiota, the stool can be homogenized and filtered from rough particulate matter. In one aspect, the microscopic fiber/nonliving matter is then separated from the bacteria. Several methods can be used, including e.g., recurrent filtration with filter sizes, e.g., progressively coming down to the size of a typical bacterium.

In one aspect, different filters are used to isolate bacterial sp., or a technique as used by Williams in WO 2011/033310A1 (hereby incorporated herein by reference in its entirety), which uses a crude technique of filtration with a gauze.

In one aspect, a filtration procedure for filtering whole stool is suitably used to reach the highest concentration of almost 100% bacteria. In one aspect, the filtering procedure is a two-step procedure suitably using glass fibre depth filters for initial clarification. In one aspect, the stool is filtered under positive pressure. In one aspect, this would be using a combination or sandwich configuration with a 30 micron PVDF filter. In one aspect, this sandwich procedure will be filtering the product under positive pressure. Later, membrane concentration can, in one aspect, be used as another step to reduce the volume of the filtrate. In one aspect, this can be done prior to freeze drying or spray drying under nitrogen cover.

Alternative membranes that can be used for filtration include, but not limited to, nylon filters, cellulose nitrate filters, polyethersulfone (PES) filters, polytetrafluorethylene (PTFE) filters, TEFLON™ filters, mixed cellulose Ester filters, polycarbonate filters, polypropylene filters, Polyvinylchloride (PVC) filters or quartz filters. Various combinations of these can be used to achieve a high purity of bacteria with solids and liquid removed.

### Pharmaceutical Compositions, Formulations, and Administration

Described herein are pharmaceutical compositions comprising a bacterial mixture comprising a preparation of uncultured fecal bacteria and/or one or more bacterial isolates in various formulations. Any pharmaceutical composition described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the pharmaceutical compositions can conveniently be presented in unit dosage forms. For example, the dosage forms can be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting).

In another aspect, a pharmaceutical composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and a combination thereof. In another aspect, a pharmaceutical composition can contain auxiliary substances such as wetting or emulsifying agents, stabilizing or pH buffering agents. In an aspect, a pharmaceutical composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a pharmaceutical composition contains about 2%-70%, 5%-60%, 10%-50%, 15%-40%, 20%-30%, 25%-60%, 30%-60%, or 35%-60% of active ingredient.

In an aspect, a pharmaceutical composition can be incorporated into tablets, drenches, boluses, capsules, premixes or patches. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, for preparing solid compositions such as tablets, an active ingredient is mixed with a pharmaceutical carrier, e.g., conventional tableting ingredients such as cornstarch, lactose, sucrose, sorbitol, talc, stearic acid, magnesium stearate, dicalcium phosphate or gums, or other pharmaceutical diluents, e.g. water, to form a solid pre-formulation composition containing a homogeneous mixture of a composition described herein. When referring to these pre-formulation compositions as homogeneous, it is meant that the active ingredient is dispersed evenly throughout the composition so that the composition can be readily subdivided into equally effective unit dosage forms such as tablets, pills and capsules. This solid pre-formulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ CFUs). A pharmaceutical composition described herein can be flavored.

In an aspect, a pharmaceutical composition comprising a bacterial mixture described herein (and optionally one or more additional therapeutic agents) is formulated as a composition adapted for a mode of administration described herein.

In various aspects, the administration of a pharmaceutical composition is any one of oral, intravenous, intraperitoneal, and parenteral. For example, routes of administration include, but are not limited to, oral, intraperitoneal, intravenous, intramuscular, or rectally. In various aspects, the administration of the pharmaceutical compositions is oral, naso-gastric, antegrade gastrointestinal, retrograde gastrointestinal, endoscopic, or enemic.

In an aspect, a pharmaceutical composition described herein can be formulated as a composition adapted for oral administration. Compositions for oral delivery can be in the form of tablets, lozenges, aqueous or oily suspensions, granules, powders, sprinkles, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration to provide sustained delivery of the bacterial mixture over an extended period of time. Selectively permeable membranes surrounding an osmotically active agent are also suitable for orally administered compositions. In these latter platforms, fluid from the environment surrounding the capsule is imbibed by a driving compound, which swells to displace the agent or agent composition through an aperture. These delivery platforms can provide an essentially zero order delivery profile as opposed to the spiked profiles of immediate release formulations. A time-delay material, such as glycerol monostearate or glycerol stearate, can also be useful. Oral compositions can include standard excipients such as mannitol, lactose, starch, magnesium stearate, sodium saccharin, cellulose, ethacrylic acid and derivative polymers thereof, and magnesium carbonate. In an aspect, the excipients are of pharmaceutical grade. Suspensions, in addition to the active compounds, can contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In various aspects, a pharmaceutical composition is formulated as a solid dosage form such as a tablet, dispersible powder, granule, or capsule. In an aspect, the pharmaceutical composition is formulated as a capsule. In another aspect, the pharmaceutical composition is formulated as a tablet. In yet another aspect, the pharmaceutical composition os formulated as a soft-gel capsule. In a further aspect, the pharmaceutical composition is formulated as a gelatin capsule.

In an aspect, a pharmaceutical composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a pharmaceutical composition described herein is formulated in in the form of microcapsules. Microencapsulation is the coating of a liquid or solid with a protective wall material that inhibits volatilization and protects against chemical deterioration. The solid or liquid contained within the wall material is known as the core, and the complete microencapsulated particle is known as a microcapsule. Wall materials that can be used here include gum arabic, carboxymethyl cellulose, alginate, gelatin, whey protein, sodium caseinate, and soy protein.

In an aspect, a mixture of (a) a preparation of uncultured fecal bacteria and (b) one or more bacterial isolates are co-encapsulated in a same pool of microcapsules and administered to a subject. In another aspect, a preparation of uncultured fecal bacteria are microencapsulated separately from one or more bacterial isolates and administered to a subject either separately (e.g., sequentially) or together (e.g., after mixing microcapsules having different encapsulated contents). In a further aspect, bacteria from each different bacterial isolate are microencapsulated separately and the resulting microcapsules are subsequently mixed together, for administering a subject, according to a desired ratio or proportion of each of the bacterial isolates (with or without separate microcapsules containing a preparation of uncultured fecal bacteria).

Microencapsulation can be performed with a microencapsulation device, including microfluidic droplet generation or encapsulation devices. An exemplary microencapsulation device is described, for example, in U.S. Pat. No. 7,482,152, hereby incorporated herein by reference in its entirety. Microcapsules can comprise one or more stabilizers or gelling agents, which can be used to stabilize a microcapsule or emulsion. Stabilizers or gelling agents can include but are not limited to alginate (also algin or alginic acid) and agar. Alginate can be used in a variety of forms, including but not limited to inorganic salts such as sodium alginate, potassium alginate, calcium alginate, and combinations thereof. Alginate can be derived from sources such as seaweed (e.g., Macrocystis pyrifera, Ascophyllum nodosum, Laminaria spp.) or bacteria (e.g., Pseudomonas spp., Azotobacter spp.). Cross-linking agents or solutions, such as calcium chloride, can be used to stabilize or gel microcapsules. In an aspect, alginate-based microcapsules are made and used according to US20160317583. In an aspect, an alginate polymer has a concentration of about 2.5% (w/v) wherein the microcapsule comprises an additional sequential external surface coating of poly-L-lysine. In a further aspect, microcapsule comprises a divalent cation from the group of calcium or barium or a mixture of calcium and barium to crosslink the alginate polymer into a microcapsule.

Microcapsules can be characterized by a size (e.g., a diameter). The microcapsule size can be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 millimeters. The microcapsule size can be less than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 millimeters. The microcapsule size can be greater than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 millimeters. The microcapsule size can be from about 0.05 to about 1 millimeters. The size distribution in a population of microcapsules can be homogeneous or substantially homogeneous. For example, a population of microcapsules can be characterized by dispersity, or polydispersity index (PDI), of less than or equal to about 20, 19, 18, 17, 16, 16, 15, 14, 13, 12, 11, 10, 9, 8, 7, 6, 5, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.45, 1.40, 1.35, 1.30, 1.25, 1.20, 1.15, 1.14, 1.13, 1.12, 1.11, 1.10, 1.09, 1.08, 1.07, 1.06, 1.05, 1.04, 1.03, 1.02, 1.01, or 1.00.

In various aspects, formulations can additionally comprise a pharmaceutically acceptable carrier or excipient. As one skilled in the art will recognize, the formulations can be in any suitable form appropriate for the desired use and route of administration.

In some dosage forms, a pharmaceutical composition described herein is mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients can have two or more functions in the oral dosage form. In the case of an oral dosage form, for example, a capsule or a tablet, the dosage form can also comprise buffering agents.

In an aspect, a pharmaceutical composition comprising a bacterial mixture is combined with one or more pharmaceutically acceptable cryoprotectants, lyoprotectants, binders, disintegrants, excipients, fillers, and/or preservatives, acid suppressants, antacids, H2 antagonists, and proton pump inhibitors, or combinations thereof. In an aspect, a pharmaceutical composition comprising a bacterial mixture is combined with one or more pharmaceutically acceptable agents for taste masking (e.g., taste-maskers). In an aspect, a pharmaceutical composition comprising a bacterial mixture is combined with one or more pharmaceutically acceptable mixable powders to improve color attractiveness, taste, and/or flavor, for easier administering in ASD patients, especially in children ASD patients. Exemplary taste-masker include Smoothenol. Exemplary flavors include peach, orange, strawberry, grape, papaya, mixed berry, dark chocolate, pineapple, and blueberry.

In an aspect, a pharmaceutical composition comprising a bacterial mixture is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a pharmaceutical composition.

In one aspect, a pharmaceutical composition administered herein further comprises an acid suppressant, an antacid, an H2 antagonist, a proton pump inhibitor or a combination thereof. In one aspect, a pharmaceutical composition administered herein substantially free of non-living matter. In another aspect, a pharmaceutical composition administered herein substantially free of acellular material selected from the group consisting of residual fiber, DNA, viral coat material, and non-viable material. In another aspect, a pharmaceutical composition administered does not comprise an acid suppressant, an antacid, an H2 antagonist, a proton pump inhibitor or a combination thereof. In yet another aspect, a pharmaceutical composition administered does not comprise an acid suppressant. In another aspect, a pharmaceutical composition administered does not comprise an antacid. In another aspect, a pharmaceutical composition administered does not comprise an H2 antagonist. In another aspect, a pharmaceutical composition administered does not comprise a proton pump inhibitor. In another aspect, a pharmaceutical composition administered does not comprise metoclopramide.

In an aspect, a bacterial mixture is dry, e.g., when it includes lyophilized bacterial cells/spores or comprises dry binders, fillers, and dispersants. Alternately, the bacterial mixture comprising can be is aqueous, e.g., when it comprises non-dry binders, fillers, and dispersants.

In an aspect, a bacterial mixture described herein can be subject to lyophilization. As used herein, “lyophilization” or “freeze drying” refers to the process of drying a material by first freezing it and then encouraging the ice within it to sublimate in a vacuum environment.

In one aspect, a bacterial mixture comprises a lyophilized formulation further comprising a reducing agent and/or antioxidant. In certain aspects, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfate, sodium metabisulfite, potassium metabisulfite, glutathione, methionine, thioglycerol, and alpha tocopherol.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In one aspect, a bacterial mixture comprises a cryoprotectant or mixture of cryoprotectants. As used herein, a “cryoprotectant” refers to a substance that is added to a formulation in order to protect an active ingredient during freezing. For example, a cryoprotectant can comprise, consist essentially of, or consist of polyethylene glycol, skim milk, erythritol, arabitol, sorbitol, glucose, fructose, alanine, glycine, proline, sucrose, lactose, ribose, trehalose, dimethyl sulfoxide (DMSO) or equivalent, a glycerol, a polyethylene glycol (PEG) or equivalent, or an amino acid (e.g., alanine, glycine, proline). In an aspect of the present disclosure, a cryoprotectant can be selected from the group comprising 5% sucrose; 10% sucrose; 10% skim milk; 10% trehalose with 2.5% sucrose; 5% trehalose with 2.5% sucrose; 5% mannitol; 5% mannitol with 0.1% polysorbate 80; 10% mannitol; 10% mannitol with 0.1% polysorbate 80; 5% trehalose; 5% trehalose with 0.1% polysorbate 80; 10% trehalose; and 10% trehalose with 0.1% polysorbate 80.

In an aspect, a bacterial mixture comprises a lyoprotectant. As used herein, a “lyoprotectant” refers to a substance that is added to a formulation in order to protect an active ingredient during the stage of a lyophilization (also known as freeze-drying). In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and a combination thereof. In an aspect, a lyoprotectant is a non-reducing sugar, such as trehalose or sucrose. In an aspect, a cryoprotectant or a lyoprotectant consists essentially of, or consists of, one or more substances mentioned in this paragraph and the paragraph above.

In an aspect, a cryoprotectant or a lyoprotectant comprise an intracellular agent, e.g., DMSO, glycerol, or PEG, which penetrates inside the cell preventing the formation of ice crystals that could result in membrane rupture. In an aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, the present disclosure provides a pharmaceutical composition comprising a lyophilized fecal microbe preparation comprising a lyophilization formulation comprising at least about 12.5% trehalose.

In an aspect, a lyophilized formulation comprises trehalose. In an aspect, a lyophilized formulation comprises 2% to 30%, 3% to 25%, 4% to 20%, 5% to 15%, 6% to 10%, 2% to 30%, 2% to 25%, 2% to 20%, 2% to 15%, or 2% to 10% trehalose. In an aspect, a lyophilized formulation comprises at least 2%, 3%, 4%, 5%, 6%, 7%, 8%, 9%, 10%, or 15% trehalose. In an aspect, a lyophilized formulation comprises at most 2%, 3%, 4%, 5%, 6%, 7%, 8%, 9%, 10%, or 15% trehalose. In another aspect, a lyophilized formulation comprises about 5% trehalose. In another aspect, a lyophilized formulation comprises trehalose and sucrose. In another aspect, a lyophilized formulation comprises between about 8% and 12% trehalose with between about 1.5% and 3.5% sucrose and between about 0.5% and 1.5% NaCl.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In an aspect, a pharmaceutical composition provided here, after at least 12 weeks of storage at ambient temperature or lower, is effective for treating a subject having ASD. In an aspect, a pharmaceutical composition remains effective after at least 4, 8, 10, 16, 20, 24, 30, 40, 50, 60, 70, 80 or 100 weeks of storage at ambient temperature or lower.

In an aspect, a pharmaceutical composition described herein can be lyophilized or freeze dried and stored at ambient temperatures (e.g., room temperature), at a freezing temperature, or at between about 2° C. and 8° C. In an aspect, freeze-drying allows the majority of cells to remain viable, and produces a powdered form of the product that can be gently pulverized into a powder. The powder, or lyophilized or freeze-dried composition, then can be encapsulated into a carrier, e.g., a tablet, geltab, pill or capsule, e.g., an enteric-coated capsule, or placed into oil-filled capsules for ingestion. Alternatively, the freeze-dried or lyophilized product, or powder, can be reconstituted at ambient temperatures before delivery to an individual in e.g., a fluid, e.g., a sterile fluid, such as saline, a buffer or a media such as a fluid-glucose-cellobiose agar (RGCA) media.

For freeze-drying, in an aspect, bacteria are held in a liquid that will prevent bursting of cells on thawing. This can include various stabilizers, e.g., glycerol and appropriate buffers, and/or ethylene glycol. In an aspect, the cryoprotecting process uses final concentrations of stabilizer(s) of between about 10% and 80%, 20% and 70%, 30% and 60%, or 40% and 50%, depending on the stabilizer(s) used; in an aspect, this helps stabilize proteins by preventing formation of ice crystals that would otherwise destroy protein structures.

In an aspect, stabilizers that help reduce destruction of living bacteria include skim milk, erythritol, arabitol, sorbitol, glucose, fructose and other polyols. Polymers such as dextran and polyethylene glycol can also be used to stabilize bacterial cells.

In an aspect, manufacturing a pharmaceutical composition can comprise steps of: (1) coating the exterior of a dissociated capsule (i.e., comprising separate capsule body and capsule cap) with the exterior enteric coating, (2) filling the capsule body with a bacterial mixture (e.g., comprising one or more bacterial isolates and/or a preparation of uncultured fecal bacteria), and (3) closing the capsule cap over the capsule body, thereby encapsulating the bacterial mixture in the enteric-coated capsule.

Optionally, manufacturing a pharmaceutical composition can comprise steps of: (1) coating the exterior of a dissociated capsule (i.e., comprising separate capsule body and capsule cap) with the exterior enteric coating, (2) coating the interior of the dissociated capsule with an interior coating, (3) filling the capsule body with a bacterial mixture (e.g., comprising one or more bacterial isolates and/or a preparation of uncultured fecal bacteria), and (4) closing the capsule cap over the capsule body, thereby encapsulating the bacterial mixture in the dual-coated capsule.

Alternately, manufacturing a pharmaceutical composition can comprise step of: (1) coating the interior of the dissociated capsule (i.e., comprising separate capsule body and capsule cap) with an interior coating, (2) coating the exterior of a dissociated capsule with the exterior enteric coating, (3) filling the capsule body with a bacterial mixture (e.g., comprising one or more bacterial isolates and/or a preparation of uncultured fecal bacteria), and (4) closing the capsule cap over the capsule body, thereby encapsulating the bacterial mixture in the dual-coated capsule.

In an aspect, one or more additional therapeutic agents can be included in a pharmaceutical composition, and encapsulated by the capsule.

In an aspect, the bodies and caps of gelatin capsules (e.g., size #00) are separated. An exterior enteric coating suspension is prepared by dispersing one or more enteric coating polymers along with other components in a solution. The exterior enteric coating suspension is applied to the exterior of separated capsule bodies and caps, e.g., using a fluid bed Wurster column coater, Fluid Bed Coater, or an equivalent). The capsules are fluidized in the product bowl and the exterior enteric coating suspension is sprayed to produce the outer coating to a target of between about 2 mg/cm2 and 6 mg/cm2, e.g., 3 mg/cm2. After completion of this step, the capsules are set to dry, e.g., between about 8 hours and 24 hours. After drying, exemplary capsules are weighed to calculate weight gain from the exterior enteric coating. Capsules can be inspected for irregularities.

In an aspect, EUDRAGIT® S100 (poly(methacrylic acid, methylmethacrylate)), starch, triethyl citrate, and PlasACRYL® T20 are dissolved in a solution of water, ethanol, and n-butanol, mixed, and then charged to a suitable spraying device. The solution is then spray coated on the outer surface of the capsule bodies and capsule caps to a target weight gain. The capsule bodies and capsule caps are allowed to dry for about 8 hours to about 24 hours, or longer, e.g., for a week, a month, or more, before further processing, e.g., filling with a bacterial mixture.

In an aspect, it may be desirable to provide an amount of the bacterial mixture to a capsule's cap in addition to providing the composition in the capsule's body. In this aspect, more of the composition will be included in a capsule and/or less air will be contained in a closed capsule.

In an aspect, the interior surface of a capsule comprises an internal coating, for example an internal coating that is water insoluble.

Any of the above-described compositions and materials (e.g., bacterial mixtures, inner coatings, capsules, and outer coatings) can be combined into a pharmaceutical composition described herein. A skilled artisan would know how to select an inner coating; capsule, and outer coating according to his/her present need, which could be based, for example, on a specific bacterial isolate(s) incorporated into the composition and/or the desired delivery location in a subject (e.g., in the colon or small intestine, including the ileum, jejunum or duodenum), and where the bacterial isolate(s) should be delivered.

In an aspect, a pharmaceutical composition comprising a bacterial isolate that comprises bacteria of the genus Lactobacillus releases the bacterial isolate in the ileum of the intestine. In an aspect, the bacterial isolate comprises Lactobacillus reuteri.

Additional relevant teachings are disclosed in WO 2007122374, which is hereby incorporated herein by reference in its entirety.

In an aspect, during the manufacture of a pharmaceutical composition, a pharmaceutically-acceptable cryoprotectant, lyoprotectant, binder, disintegrant, filler, preservative, acid suppressant, antacid, H2 antagonist, and proton pump inhibitor, or combination thereof can be mixed into the pharmaceutical composition (e.g., comprising a bacterial mixture) to promote desirable properties.

In an aspect, the pharmaceutical composition comprises a surface active agent. Surface active agents suitable for use include, but are not limited to, any pharmaceutically acceptable, non-toxic surfactant. Classes of surfactants suitable for use include, but are not limited to, polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some aspects, compositions can comprise one or more surfactants including, but not limited to, sodium lauryl sulfate, polysorbate 20, polysorbate 40, polysorbate 60, polysorbate 80, and triethyl citrate.

In an aspect, the pharmaceutical composition comprises pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

In another aspect, the pharmaceutical composition comprises one or more application solvents. Some of the more common solvents that can be used to apply, for example, a delayed-release coating composition include isopropyl alcohol, acetone, methylene chloride and the like.

In yet another aspect, the pharmaceutical composition comprises one or more alkaline materials. Alkaline material suitable for use in compositions include, but are not limited to, sodium, potassium, calcium, magnesium and aluminum salts of acids such as phosphoric acid, carbonic acid, citric acid and other aluminum/magnesium compounds. In addition, the alkaline material can be selected from antacid materials such as aluminum hydroxides, calcium hydroxides, magnesium hydroxides and magnesium oxide.

Besides inert diluents, the orally administered compositions can also include adjuvants such as sweetening, flavoring, and perfuming agents.

In various aspects, the pharmaceutical compositions are formulated for systemic or local delivery. In an aspect, administration is systemic. In another aspect, it may be desirable to administer locally to the area in need of treatment.

Various methods can be used to formulate and/or deliver a pharmaceutical composition (e.g., comprising a bacterial mixture) described herein to a location of interest. For example, the pharmaceutical compositions can be formulated for delivery to the GI tract. The GI tract includes organs of the digestive system such as mouth, esophagus, stomach, small intestine, duodenum, jejunum, ileum, large intestine and rectum and includes all subsections thereof (e.g. the small intestine may include the duodenum, jejunum and ileum; the large intestine may include the colon transversum, colon descendens, colon ascendens, colon sigmoidenum and cecum). For example, the compositions can be formulated for delivery of one or more active agents to one or more of the stomach, small intestine, large intestine and rectum, or any subsection thereof (e.g. duodenum, jejunum and ileum, colon transversum, colon descendens, colon ascendens, colon sigmoidenum and cecum). In some aspects, the compositions described herein can be formulated to deliver to the upper or lower GI tract. In an aspect, a composition can be administered to a subject, by, for example, directly or indirectly contacting the mucosal tissues of the GI tract with the composition.

In various aspects, the administration of the pharmaceutical compositions is into the GI tract via, for example, oral delivery, nasogastral tube, intestinal intubation (e.g. an enteral tube or feeding tube such as, for example, a jejunal tube or gastro jejunal tube, etc.), direct infusion (e.g., duodenal infusion), endoscopy, colonoscopy, or enema.

In one aspect, a method comprises administering a pharmaceutical composition orally, by enema, or via rectal suppository. In one aspect, a pharmaceutical composition administered herein is formulated as an enteric coated (and/or acid-resistant) capsule or microcapsule, or formulated as part of or administered together with a food, a food additive, a dairy-based product, a soy-based product or a derivative thereof, a jelly, a gelatin-based chewable (e.g., a gummy), flavored liquid, ice block, ice cream, or a yogurt. In another aspect, a pharmaceutical composition administered herein is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

In an aspect, a pharmaceutical composition comprises a liquid culture. In another aspect, a pharmaceutical composition is homogenized, lyophilized, pulverized and powdered. It can then be infused, dissolved such as in saline, as an enema. Alternatively, the powder can be encapsulated as enteric-coated and/or acid-resistant delayed release capsules for oral administration. In an aspect, the powder can be double encapsulated with acid-resistant/delayed release capsules for oral administration. These capsules can take the form of enteric-coated and/or acid-resistant delayed release microcapsules. A powder can be provided in a palatable form for reconstitution for drinking or for reconstitution as a food additive. In a further aspect, a food is yogurt. In one aspect, a powder can be reconstituted to be infused via naso-duodenal infusion.

In another aspect, a pharmaceutical composition administered herein is in a liquid, frozen, freeze-dried, spray-dried, foam-dried, lyophilized, or powder form. In a further aspect, a pharmaceutical composition administered herein is formulated as a delayed or gradual enteric release form. In another aspect, a pharmaceutical composition administered herein comprises an excipient, a saline, a buffer, a buffering agent, or a fluid-glucose-cellobiose agar (RGCA) media. In another aspect, a pharmaceutical composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In various aspects, provided herein are modified-release formulations comprising a bacterial mixture (e.g., comprising one or more bacterial isolates in combination with a preparation of uncultured fecal bacteria), wherein the formulation releases a substantial amount of the bacterial mixture (and optionally additional therapeutic agents) into one or more regions of the GI tract. For example, the formulation can release at least about 60% of the bacterial isolates after the stomach and into one or more regions of the GI tract.

In various aspects, the modified-release formulation can release at least 60% of the bacterial mixture (and optionally additional therapeutic agents) after the stomach into one or more regions of the intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial mixture (and optionally additional therapeutic agents) in the intestines.

In various aspects, the modified-release formulation can release at least 60% of the bacterial mixture (and optionally additional therapeutic agents) in the small intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial mixture (and optionally additional therapeutic agents) in the small intestine (e.g., one or more of duodenum, jejunum, ileum, and ileocecal j unction).

In various aspects, the modified-release formulation can release at least 60% of the bacterial mixture (and optionally additional therapeutic agents) in the large intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial isolates (and/or additional therapeutic agents) in the large intestine (e.g., one or more of cecum, ascending, transverse, descending or sigmoid portions of the colon, and rectum).

‘In some aspects, the pharmaceutical composition is formulated for release in the stomach. In other aspects, the pharmaceutical composition is formulated so as to not substantially release the bacterial mixture in the stomach.

In certain aspects, the modified-release formulation releases the bacterial mixture (and optionally additional therapeutic agents) at a specific pH. For example, in some aspects, the modified-release formulation is substantially stable in an acidic environment and substantially unstable (e.g., dissolves rapidly or is physically unstable) in a near neutral to alkaline environment. In some aspects, stability is indicative of not substantially releasing while instability is indicative of substantially releasing. For example, in some aspects, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some aspects, the present formulations are stable in lower pH areas and therefore do not substantially release in, for example, the stomach. In some aspects, modified-release formulation is substantially stable at a pH of about 1 to about 4 or lower and substantially unstable at pH values that are greater. In these aspects, the modified-release formulation does not substantially release in the stomach. In these aspects, the modified-release formulation substantially releases in the small intestine (e.g. one or more of the duodenum, jejunum, and ileum) and/or large intestine (e.g. one or more of the cecum, ascending colon, transverse colon, descending colon, and sigmoid colon). In some aspects, modified-release formulation is substantially stable at a pH of about 4 to about 5 or lower and consequentially is substantially unstable at pH values that are greater and therefore is not substantially released in the stomach and/or small intestine (e.g. one or more of the duodenum, jejunum, and ileum). In these aspects, the modified-release formulation substantially releases in the large intestine (e.g. one or more of the cecum, ascending colon, transverse colon, descending colon, and sigmoid colon). In various aspects, the pH values recited herein can be adjusted as known in the art to account for the state of the subject, e.g. whether in a fasting or postprandial state.

In some aspects, the modified-release formulation is substantially stable in gastric fluid and substantially unstable in intestinal fluid and, accordingly, is substantially released in the small intestine (e.g. one or more of the duodenum, jejunum, and ileum) and/or large intestine (e.g. one or more of the cecum, ascending colon, transverse colon, descending colon, and sigmoid colon).

In some aspects, the modified-release formulation is stable in gastric fluid or stable in acidic environments. These modified-release formulations release about 30% or less by weight of the pharmaceutical composition (e.g., comprising a bacterial mixture) in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of can release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the composition in the modified-release formulation in gastric fluid with a pH of 4-5, or less or simulated gastric fluid with a pH of 4-5 or less, in about 15, or about 30, or about 45, or about 60, or about 90 minutes. Modified-release formulations can release about 1%, about 2%, about 3%, about 4%, about 5%, about 6%, about 7%, about 8%, about 9%, or about 10% by weight of the total composition in the modified-release formulation in gastric fluid with a pH of 5 or less, or simulated gastric fluid with a pH of 5 or less, in about 15, or about 30, or about 45, or about 60, or about 90 minutes.

In some aspects, the modified-release formulation is unstable in intestinal fluid. These modified-release formulations release about 70% or more by weight of the bacterial mixture and/or additional therapeutic agent in the modified-release formulation in intestinal fluid or simulated intestinal fluid in about 15, or about 30, or about 45, or about 60, or about 90 minutes. In some aspects, the modified-release formulation is unstable in near neutral to alkaline environments. These modified-release formulations release about 70% or more by weight of the bacterial mixture and/or additional therapeutic agent in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments can release 70% or more by weight of the pharmaceutical composition (e.g., comprising a microbial cocktail) in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In various aspects, the modified-release formulation can be substantially stable in chyme. For example, there is, in some aspects, a loss of less about 50% or about 40%, or about 30%, or about 20%, or about 10% of the activity or viability of the bacteria in the bacterial mixture in about 10, or 9, or 8, or 7, or 6, or 5, or 4, or 3, or 2, or 1 hour from administration.

In various aspects, the modified-release formulations can be designed for immediate release (e.g. upon ingestion). In various aspects, the modified-release formulations can have sustained-release profiles, i.e. slow release of the active ingredient(s) in the body (e.g., GI tract) over an extended period of time. In various aspects, the modified-release formulations can have a delayed-release profile, i.e. not immediately release the active ingredient(s) upon ingestion; rather, postponement of the release of the active ingredient(s) until the composition is lower in the GI tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine.

In various aspects, the modified-release formulations can utilize one or more modified-release coatings such as delayed-release coatings to provide for effective, delayed yet substantial delivery of the bacterial mixture to the GI tract together with, optionally, additional therapeutic agents.

In an aspect, the delayed-release coating includes an enteric agent that is substantially stable in acidic environments and substantially unstable in near neutral to alkaline environments. In an aspect, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymers include, for example, EUDRAGIT® FS 30D, L 30 D-55, L 100-55, L 100, L 12.5, L 12.5 P, RL 30 D, RL PO, RL 100, RL 12.5, RS 30 D, RS PO, RS 100, RS 12.5, NE 30 D, NE 40 D, NM 30 D, S 100, S 12.5, and S 12.5 P. Similar polymers include Kollicoat® MAE 30 DP and Kollicoat® MAE 100 P. In some aspects, one or more of EUDRAGIT® FS 30D, L 30 D-55, L 100-55, L 100, L 12.5, L 12.5 P RL 30 D, RL PO, RL 100, RL 12.5, RS 30 D, RS PO, RS 100, RS 12.5, NE 30 D, NE 40 D, NM 30 D, S 100, S 12.5 S 12.5 P, Kollicoat® MAE 30 DP and Kollicoat® MAE 100 P is used. In various aspects, the enteric agent can be a combination of the foregoing solutions or dispersions.

In certain aspects, one or more coating system additives are used with the enteric agent. For example, one or more PlasACRYL™ additives can be used as an anti-tacking agent coating additive. Illustrative PlasACRYL™ additives include, but are not limited to, PlasACRYL™ HTP20 and PlasACRYL™ T20.

In another aspect, the delayed-release coating can degrade as a function of time when in aqueous solution without regard to the pH and/or presence of enzymes in the solution. Such a coating can comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings can be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers can include polyvinyl esters, polyvinyl acetals, polyacrylic acid esters, butadiene styrene copolymers, and the like. In an aspect, colonic delivery is achieved by use of a slowly eroding wax plug (e.g., various PEGS, including for example, PEG6000).

In a further aspect, the delayed-release coating can be degraded by a microbial enzyme present in the gut flora. In an aspect, the delayed-release coating can be degraded by bacteria present in the small intestine. In another aspect, the delayed-release coating can be degraded by bacteria present in the large intestine.

In various aspects, the modified release formulation can be designed for release in the colon. Various colon-specific delivery approaches can be utilized. For example, the modified release formulation can be formulated using a colon-specific drug delivery system (CODES) as described for example, in Li et al., AAPS PharmSciTech (2002), 3(4): 1-9, the entire contents of which are incorporated herein by reference. Drug release in such a system is triggered by colonic microflora coupled with pH-sensitive polymer coatings. For example, the formulation can be designed as a core tablet with three layers of polymer. The first coating is an acid-soluble polymer (e.g., EUDRAGIT E), the outer coating is enteric, along with a hydroxypropyl methylcellulose barrier layer interposed in between. In another aspect, colon delivery can be achieved by formulating the pharmaceutical composition (e.g., comprising a microbial cocktail) with specific polymers that degrade in the colon such as, for example, pectin. The pectin can be further gelled or crosslinked with a cation such as a zinc cation. In an aspect, the formulation is in the form of ionically crosslinked pectin beads which are further coated with a polymer (e.g., EUDRAGIT polymer). Additional colon specific formulations include, but are not limited to, pressure-controlled drug delivery systems (prepared with, for example, ethylcellulose) and osmotic controlled drug delivery systems (i.e., ORDS-CT).

Formulations for colon specific delivery of the bacterial mixture (and optionally additional therapeutic agents), as described herein, can be evaluated using, for example, in vitro dissolution tests. For example, parallel dissolution studies in different buffers can be undertaken to characterize the behavior of the formulations at different pH levels. Alternatively, in vitro enzymatic tests can be carried out. For example, the formulations can be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In a further aspect, in vivo evaluations can be carried out using animal models such as dogs, guinea pigs, rats, and pigs. Further, clinical evaluation of colon specific drug delivery formulations can be evaluated by calculating drug delivery index (DDI) which considers the relative ratio of RCE (relative colonic tissue exposure to the drug) to RSC (relative amount of drug in blood i.e. that is relative systemic exposure to the drug). Higher drug DDI indicates better colon drug delivery. Absorption of drugs from the colon can be monitored by colonoscopy and intubation.

In various aspects, the present formulation provides for substantial uniform delivery of the bacterial mixture (and optionally additional therapeutic agents) in the area of release in the GI tract. In an aspect, the present formulation minimizes patchy or heterogeneous release of the bacterial mixture.

In various aspects, the present formulations provide for release of multiple doses of one or more bacterial mixtures along the GI tract. For example, the composition and/or formulation can release multiple doses of the same bacterial mixture at different locations along the intestines, at different times, and/or at different pH. Alternatively, the composition and/or formulation can release a dose of different bacterial mixtures at different locations along the intestines, at different times, and/or at a different pH. In an aspect, the pharmaceutical composition comprises a first bacterial mixture comprising one or more bacterial isolates that is released at a first location in the intestine, and a second bacterial mixture comprising a preparation of uncultured fecal bacteria that is released at a second location in the intestine. In an aspect, the first bacterial mixture is released in the ileum, and the second bacterial mixture is released in the colon.

The overall release profile of such a formulation can be adjusted using, for example, multiple particle types or multiple layers. For example, in an aspect, a first bacterial mixture (or first dose of a bacterial mixture) can be formulated for release in, for example, the small intestine (e.g., one or more of duodenum, jejunum, ileum), whereas the second bacterial mixture (or second dose of the bacterial mixture) is formulated for delayed release in, for example, the large intestine (e.g., one or more of cecum, ascending, transverse, descending or sigmoid portions of the colon, and rectum). In another example, the first bacterial mixture (or first dose of a bacterial mixture) can be formulated for release in, for example, the small intestine (e.g., one or more of duodenum, jejunum, ileum), whereas the second bacterial mixture (or second dose of a bacterial mixture) is formulated for delayed release in, for example, another part of the small intestine (e.g., one or more of duodenum, jejunum, ileum). In another aspect, the first bacterial mixture (or first dose of a bacterial mixture) can be formulated for release in, for example, the large intestine (e.g., one or more of cecum, ascending, transverse, descending or sigmoid portions of the colon, and rectum), whereas the second bacterial mixture (or second dose of the bacterial mixture) is formulated for delayed release in, for example, another part of the large intestine (e.g., one or more of cecum, ascending, transverse, descending or sigmoid portions of the colon, and rectum). In various aspects, the composition and/or formulation can release at least one dose, at least two doses, at least three doses, at least four doses, or at least five doses of the bacterial mixture at different locations along the intestines, at different times, and/or at different pH. Likewise, in various aspects, the composition and/or formulation can release at least one bacterial mixture, at least two bacterial mixtures, at least three bacterial mixtures, at least four bacterial mixtures, or at least five bacterial mixtures at different locations along the intestines, at different times, and/or at different pH.

In another aspect, a delayed or gradual enteric release formulation comprises the use of a bilayer tablet or capsule which comprises a first layer comprising a polyalkylene oxide, a polyvinylpyrrolidone, a lubricant, or a mixture thereof, and a second osmotic push layer comprising polyethylene oxide, carboxy-methylcellulose, or both. In an aspect, a delayed or gradual enteric release formulation comprises the use of a release-retarding matrix material selected from the group consisting of an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, poly inylalcohols, polyvinylalcohol copolymers, polyethylene glycols, non-crosslinked polyvinylpyrrolidone, polyvinylacetates, polyvinylacetate copolymers, or any combination thereof. In an aspect, a delayed or gradual enteric release formulation comprises the use of a microenvironment pH modifier.

It will be understood that a pharmaceutical composition described herein can comprise multiple distinct bacterial mixtures, for example to achieve different delivery location profiles for each bacterial mixture. In an aspect, a pharmaceutical composition comprises at least two bacterial mixtures, such that a first bacterial mixture comprises one or more bacterial isolates and a second bacterial mixture comprises a preparation of uncultured fecal bacteria. In an aspect, the second bacterial mixture further comprises one or more bacterial isolates that are different than the bacterial isolates in the first bacterial mixture. Alternatively, the second bacterial mixture can consist essentially of the preparation of uncultured fecal bacteria. In another aspect, the first bacterial mixture comprises only one bacterial isolate. A pharmaceutical composition can comprise any number of bacterial mixtures, for example one, two, three, four, five, six, seven, eight, nine, ten, or more than ten bacterial mixtures that each contain a different bacterial isolate, a different combination of bacterial isolates, a preparation of uncultured fecal bacteria, or a different combination of a preparation of uncultured fecal bacteria with one or more bacterial isolates.

In an aspect, a pharmaceutical composition can be a drench. In one aspect, a drench is prepared by choosing a saline-suspended form of a pharmaceutical composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a pharmaceutical composition can be administered by a patch.

In some aspects, the bacterial isolates described herein are in the form of live, vegetative cells. In some aspects, the bacterial isolates described herein are in the form of spores. In some aspects, the bacterial isolates described herewith are lyophilized. By way of non-limiting example, lyophilization can be via methods known in the art, including those described in U.S. Pat. No. 7,799,328, the contents of which are hereby incorporated by reference in their entirety. In some aspects, lyophilized bacterial mixtures described herein are placed in an enterically coated soft gel or capsule.

In various aspects, formulations can take the form of those described in one or more of U.S. Pat. Nos. 8,535,713 and 8,911,777 and US Patent Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In various aspects, formulations can take the form of those as described in International Patent Publication No. WO 2008/135090, the contents of which are hereby incorporated by reference in their entirety.

In various aspects, formulations can take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and US Patent Publication Nos. 2014/0302132; 2014/0227357; 20140088202; 20130287842; 2013/0295188; 2013/0307962; and 20130184290, the contents of which are hereby incorporated by reference in their entirety.

### Administration and Dosage

It will be appreciated that the dose of a pharmaceutical composition or the bacterial cells therein (e.g., a bacterial mixture comprising one or more bacterial isolates and/or a preparation of uncultured fecal bacteria) will vary according to, for example, the particular dosage form, the mode of administration to a subject, the identity of a bacterial isolate, if any, in the composition, the number of bacterial isolates, if any, in the composition, and the presence or absence of a preparation of uncultured fecal bacteria in the composition. These factors, as well as variables that may modify the activity of the bacteria in a bacterial mixture (e.g., subject body weight, sex and diet, time of administration, route of administration, rate of excretion, condition of the subject, drug combinations, genetic disposition and reaction sensitivities) can be taken into account by those skilled in the art to generate an effective dose or dosage regime for treatment or prevention of at least one symptom of ASD in a patient. Administration can be carried out continuously or in one or more discrete doses within the maximum tolerated dose. Optimal administration rates for a given set of conditions can be ascertained by those skilled in the art using conventional dosage administration tests.

In various aspects, the dose of the pharmaceutical composition or the bacterial cells therein (e.g., a bacterial mixture comprising one or more bacterial isolates and/or a preparation of uncultured fecal bacteria) is effective to modulate a patient's microbiome to favor an ecological balance, so as to treat or prevent one or more symptoms of ASD.

In one aspect, a pharmaceutically active or therapeutically effective dose of a bacterial isolate administered to a subject (i.e., in single or multiple administrations) to treat at least one symptom of ASD comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ CFUs of the bacterial isolate. In another aspect, a pharmaceutically active or therapeutically effective dose of a bacterial isolate administered to a subject (i.e., in single or multiple administrations) to treat at least one symptom of ASD comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ CFUs of the bacterial isolate. In a further aspect, a pharmacologically active or therapeutically effective dose of a bacterial isolate administered to a subject (i.e., in single or multiple administrations) to treat at least one symptom of ASD is selected from the group consisting of: from 10⁸ CFUs to 10¹⁴ CFUs, from 10⁹ CFUs to 10¹³ CFUs, from 10¹⁰ CFUs to 10¹² CFUs, from 10¹⁰ CFUs to 10¹¹ CFUs, from 10⁹ CFUs to 10¹⁴ CFUs, from 10⁹ CFUs to 10¹² CFUs, from 10⁹ CFUs to 10¹¹ CFUs, from 10⁹ CFUs to 10¹⁰ CFUs, from 10¹⁰ CFUs to 10¹⁴ CFUs, from 10¹⁰ CFUs to 10¹³ CFUs, from 10¹¹ CFUs to 10¹⁴ CFUs, from 10¹¹ CFUs to 10¹³ CFUs, from 10¹² CFUs to 10¹⁴ CFUs, and from 10¹³ CFUs to 10¹⁴ CFUs of the bacterial isolate.

In an aspect, a pharmaceutical composition comprises one or more bacterial isolates, with each bacterial isolate present in each unit dose at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutically effective dose of a bacterial isolate administered to a subject (i.e., in single or multiple administrations) to treat at least one symptom of ASD comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of the bacterial isolate. In another aspect, a pharmaceutically active or therapeutically effective dose of a bacterial isolate administered to a subject i.e. in single or multiple administrations) to treat at least one symptom of ASD comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ total cells or spores of the bacterial isolate. In a further aspect, a pharmacologically active or therapeutically effective dose of a bacterial isolate administered to a subject (i.e., in single or multiple administrations) to treat at least one symptom of ASD is selected from the group consisting of: from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores of the bacterial isolate.

In an aspect, the pharmaceutically active or therapeutically effective dose cell count of a bacterial isolate is directed to live cells. In one aspect, a pharmaceutical composition comprises one or more bacterial isolates, with each bacterial isolates present in each dosage unit at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In an aspect, a pharmaceutical composition described herein is in the form of a capsule, and each capsule comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of a bacterial isolate. In an aspect, a pharmaceutical composition described herein is in the form of a capsule, and each capsule comprises from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, or from 10¹³ to 10¹⁴ cells or spores of a bacterial isolate.

In one aspect, a pharmaceutically active or therapeutically effective dose of a preparation of uncultured fecal bacteria administered to a subject (i.e., in single or multiple administrations) to treat at least one symptom of ASD comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ CFUs of the preparation of uncultured fecal bacteria. In another aspect, a pharmaceutically active or therapeutically effective dose of a preparation of uncultured fecal bacteria administered to a subject (i.e., in single or multiple administrations) to treat at least one symptom of ASD comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ CFUs of the preparation of uncultured fecal bacteria. In a further aspect, a pharmacologically active or therapeutically effective dose of a preparation of uncultured fecal bacteria administered to a subject (i.e., in single or multiple administrations) to treat at least one symptom of ASD is selected from the group consisting of: from 10⁸ CFUs to 10¹⁴ CFUs, from 10⁹ CFUs to 10¹³ CFUs, from 10¹⁰ CFUs to 10¹² CFUs, from 10¹⁰ CFUs to 10¹¹ CFUs, from 10⁹ CFUs to 10¹⁴ CFUs, from 10⁹ CFUs to 10¹² CFUs, from 10⁹ CFUs to 10¹¹ CFUs, from 10⁹ CFUs to 10¹⁰ CFUs, from 10¹⁰ CFUs to 10¹⁴ CFUs, from 10¹⁰ CFUs to 10¹³ CFUs, from 10¹¹ CFUs to 10¹⁴ CFUs, from 10¹¹ CFUs to 10¹³ CFUs, from 10¹² CFUs to 10¹⁴ CFUs, and from 10¹³ CFUs to 10¹⁴ CFUs of the preparation of uncultured fecal bacteria.

In an aspect, a preparation of uncultured fecal bacteria is present in each unit dose of a pharmaceutical composition at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutically effective dose of a preparation of uncultured fecal bacteria administered to a subject (i.e., in single or multiple administrations) to treat at least one symptom of ASD comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of the preparation of uncultured fecal bacteria. In another aspect, a pharmaceutically active or therapeutically effective dose of a preparation of uncultured fecal bacteria administered to a subject i.e. in single or multiple administrations) to treat at least one symptom of ASD comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ total cells or spores of the preparation of uncultured fecal bacteria. In a further aspect, a pharmacologically active or therapeutically effective dose of a preparation of uncultured fecal bacteria administered to a subject (i.e., in single or multiple administrations) to treat at least one symptom of ASD is selected from the group consisting of: from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores of the preparation of uncultured fecal bacteria.

In an aspect, the pharmaceutically active or therapeutically effective dose cell count of a preparation of uncultured fecal bacteria is directed to live cells. In one aspect, a preparation of uncultured fecal bacteria is present in each unit dose of a pharmaceutical composition at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In an aspect, a pharmaceutical composition described herein is in the form of a capsule, and each capsule comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of a preparation of uncultured fecal bacteria. In an aspect, a pharmaceutical composition described herein is in the form of a capsule, and each capsule comprises from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, or from 10¹³ to 10¹⁴ cells or spores of a preparation of uncultured fecal bacteria.

A subject can be administered one or more bacterial isolates combined with a preparation of uncultured fecal bacteria for treatment of one or more symptoms of ASD. In such cases, the bacterial isolate(s) and preparation of uncultured fecal bacteria can be administered to the subject together in the same pharmaceutical composition, or in separate compositions. Further, a pharmaceutical composition (e.g., comprising one or more bacterial isolates, a preparation of uncultured fecal bacteria, or both) can be administered to the subject in a single unit dose or multiple unit doses, for example as part of a dosage regime. In an aspect, the dosage of the preparation of uncultured fecal bacteria (e.g. measured by CFU or cell/spore count) administered to a subject is greater than the dosage of the bacterial isolate. Alternatively, the dosage of the preparation of uncultured fecal bacteria (e.g. measured by CFU or cell/spore count) administered to the subject can be less than the dosage of the bacterial isolate. In another aspect, the dosage of the preparation of uncultured fecal bacteria (e.g. measured by CFU or cell/spore count) can be about the same as the dosage of the bacterial isolate. For example, in an aspect a subject can be administered a bacterial isolate (e.g. Lactobacillus reuteri) at a dosage of about 10¹⁰ cells and a preparation of uncultured fecal bacteria at a dosage of about 10¹⁰ cells to treat or prevent one or more symptoms of ASD.

In an aspect, the number of cells of a bacterial isolate administered to a subject to treat one or more symptoms of ASD is about the same or greater than the total number of cells of a preparation of uncultured fecal bacteria administered to the subject. Alternatively, the number of cells of a bacterial isolate administered to a subject to treat one or more symptoms of ASD can be about the same or less than the total number of cells of a preparation of uncultured fecal bacteria administered to the subject.

In an aspect, a pharmaceutical composition comprises a bacterial mixture that comprises multiple bacterial isolates. In another aspect, at least two bacterial isolates are present at about the same amount or dosage (e.g., about the same number of viable cells or spores, or about the same CFUs). In another aspect, at least three bacterial isolates, at least four bacterial isolates, at least five bacterial isolates, at least six bacterial isolates, at least seven bacterial isolates, at least eight bacterial isolates, at least nine bacterial isolates, at least ten bacterial isolates, or more than ten bacterial isolates are present in the pharmaceutical composition at about the same amount or dosage (e.g., about the same number of viable cells or spores, or about the same CFUs). In another aspect, all of the bacterial isolates in a bacterial mixture are present in about the same amounts.

In an aspect, a pharmaceutical composition comprises a bacterial mixture comprising multiple bacterial isolates, and at least two of the multiple bacterial isolates are present at different amounts or dosages (e.g., different numbers of viable cells or spores, or different CFUs). In another aspect, at least three, at least four, at least five, at least six, at least seven, at least eight, at least nine, at least ten, or more than ten bacterial isolates are present in the bacterial mixture at different amounts or dosages.

A pharmaceutical composition can comprise a bacterial mixture comprising multiple bacterial isolates in combination with a preparation of uncultured fecal bacteria. In an aspect, each bacterial isolate is present in the composition at an amount or dosage that is greater than the amount or dosage of the preparation of uncultured fecal bacteria (e.g., measured as numbers of viable cells or spores, or CFUs). In another aspect, each bacterial isolate is present in the composition at an amount or dosage that is less than the amount or dosage of the preparation of uncultured fecal bacteria (e.g., measured as numbers of viable cells or spores, or CFUs). In another aspect, at least one bacterial isolate is present in the composition at an amount or dosage that is greater than the amount or dosage of the preparation of uncultured fecal bacteria, and at least one bacterial isolate is present in the composition at an amount or dosage that is less than the amount or dosage of the preparation of uncultured fecal bacteria (e.g., measured as numbers of viable cells or spores, or CFUs).

In an aspect, a pharmaceutical composition comprises one or more bacterial isolates at an amount or dosage which is at or above the minimum amount or dosage of the bacterial isolate required to be administered to a subject for engraftment of the bacterial isolate to occur in the intestine of the subject. For example, a minimum dosage of the bacterial isolate required for engraftment of the bacterial isolate into the intestine of the subject can be at least 10⁶ cells, at least 10⁷ cells, at least 10⁸ cells, at least 10⁹ cells, at least 10¹⁰ cells, at least 10¹¹ cells, or at least 10¹² cells. In an aspect a first and second bacterial isolate of a microbial cocktail engraft in the intestine of a subject at different minimal dosages or amounts, and a dosage or amount of each of the first and second bacterial isolate in the microbial cocktail varies corresponding to the respective minimal dosage or amount required for engraftment of the respective bacterial isolate.

Individual doses of the pharmaceutical composition (e.g., comprising a bacterial mixture) can be administered in unit dosage forms (e.g., tablets or capsules) containing, for example, from about 0.01 mg to about 5,000 mg, from about 0.01 mg to about 4,000 mg, from about 0.01 mg to about 3,000 mg, from about 0.01 mg to about 2,000 mg, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg of the active ingredient per unit dosage form, or from about 5 mg to about 80 mg per unit dosage form. For example, a unit dosage form can include about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg of the active ingredient, inclusive of all values and ranges therebetween.

In an aspect, the pharmaceutical composition (e.g., comprising a bacterial mixture) is administered at an amount of from about 0.01 mg to about 100 mg daily, an amount of from about 0.01 mg to about 5,000 mg daily, about 0.01 mg to about 4,000 mg daily, about 0.01 mg to about 3,000 mg daily, about 0.01 mg to about 2,000 mg daily, about 0.01 mg to about 1,000 mg daily, from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various aspects, the bacterial mixture is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg inclusive of all values and ranges therebetween.

In some aspects, a suitable dosage of the pharmaceutical composition (e.g., comprising a bacterial mixture) is in a range of about 0.01 mg/kg to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other aspects, a suitable dosage of the composition in a range of about 0.01 mg/kg to about 100 mg/kg of body weight, in a range of about 0.01 mg/kg to about 90 mg/kg of body weight, in a range of about 0.01 mg/kg to about 80 mg/kg of body weight, in a range of about 0.01 mg/kg to about 70 mg/kg of body weight, in a range of about 0.01 mg/kg to about 60 mg/kg of body weight, in a range of about 0.01 mg/kg to about 50 mg/kg of body weight, in a range of about 0.01 mg/kg to about 40 mg/kg of body weight, in a range of about 0.01 mg/kg to about 30 mg/kg of body weight, in a range of about 0.01 mg/kg to about 20 mg/kg of body weight, in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain aspects, the pharmaceutical composition (e.g., comprising a bacterial mixture) can be administered, for example, more than once daily, about once per day, about every other day, about every third day, about once a week, about once every two weeks, about once every month, about once every two months, about once every three months, about once every six months, or about once every year.

In an aspect, a pharmaceutical composition can be administered to a patient in need thereof at least once daily for at least two consecutive days. In another aspect, a pharmaceutical composition is administered at least once daily for at least 3, 4, 5, 6, 7, 8, 9, 10, 11, 12, 13, 14, or 15 consecutive days. In another aspect, a pharmaceutical composition is administered at least once daily for at least 1, 2, 3, 4, 5, 6, 7, 8, 9, 10, 11, or 12 consecutive weeks. In another aspect, a pharmaceutical composition is administered at least twice, three times, four times, or five times per week for at least 1, 2, 3, 4, 5, 6, 7, 8, 9, 10, 11, or 12 consecutive weeks. In another aspect, a pharmaceutical composition is administered at least once daily for at most 4, 5, 6, 7, 8, 9, 10, 11, 12, 13, 14, 15, 16, 17, 18, 19, or 20 consecutive days or weeks. Ina further aspect, a pharmaceutical composition is administered at least once daily for at most 1, 2, 3, 4, 5, 6, 7, 8, 9, 10, 11, or 12 consecutive weeks or months. In yet another aspect, a pharmaceutical composition is administered at least once for at least 1, 2, 3, 4, 5, 6, 7, 8, 9, 10, 11, or 12 consecutive months or years, chronically for a subject's entire life span, or an indefinite period of time.

In an aspect, a pharmaceutical composition can be administered to a patient in need thereof at least twice daily for at least two consecutive days. In an aspect, a pharmaceutical composition is administered at least twice daily for at least 3, 4, 5, 6, 7, 8, 9, 10, 11, 12, 13, 14, or 15 consecutive days. In another aspect, a pharmaceutical composition is administered at least twice daily for at least 1, 2, 3, 4, 5, 6, 7, 8, 9, 10, 11, or 12 consecutive weeks. In another aspect, a pharmaceutical composition is administered at least twice daily for at most 4, 5, 6, 7, 8, 9, 10, 11, 12, 13, 14, 15, 16, 17, 18, 19, or 20 consecutive days or week. In another aspect, a pharmaceutical composition is administered at least twice daily for at most 1, 2, 3, 4, 5, 6, 7, 8, 9, 10, 11, or 12 consecutive weeks or months. In another aspect, a pharmaceutical composition is administered at least twice for at least 1, 2, 3, 4, 5, 6, 7, 8, 9, 10, 11, or 12 consecutive months or years, chronically for a subject's entire life span, or an indefinite period of time.

In an aspect of the present disclosure, a pharmaceutical composition can be administered to a patient in need thereof at least three times daily for at least two consecutive days. In an aspect, a pharmaceutical composition is administered at least three times daily for at least 3, 4, 5, 6, 7, 8, 9, 10, 11, 12, 13, 14, or 15 consecutive days. In an aspect, a pharmaceutical composition is administered at least three times daily for at least 1, 2, 3, 4, 5, 6, 7, 8, 9, 10, 11, or 12 consecutive weeks. In an aspect, a pharmaceutical composition is administered at least three times daily for at most 4, 5, 6, 7, 8, 9, 10, 11, 12, 13, 14, 15, 16, 17, 18, 19, or 20 consecutive days or weeks. In an aspect, a pharmaceutical composition is administered at least three times daily for at most 1, 2, 3, 4, 5, 6, 7, 8, 9, 10, 11, or 12 consecutive weeks or months. In an aspect, a pharmaceutical composition is administered at least three times for at least 1, 2, 3, 4, 5, 6, 7, 8, 9, 10, 11, or 12 consecutive months or years, chronically for a subject's entire life span, or an indefinite period of time.

In an aspect, a pharmaceutical composition can be administered to a patient in need thereof at a dosing schedule of at least once or twice daily for at least three consecutive days or weeks. In an aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In an aspect, a pharmaceutical composition can be administered to a patient in need thereof at a dosing schedule of once-a-week, twice-a-week, or thrice-a-week. The term “once-a-week” means that a dose is administered typically only once in a week, for example, on the same day of each week. “Twice-a-week” means that a dose is administered typically only two times in a week, for example, on the same two days of each weekly period. “Thrice-a-week” means that a dose is administered typically only three times in a week, for example, on the same three days of each weekly period.

In an aspect, a pharmaceutical composition can be administered to a patient in need thereof, wherein the administration comprises a first dosing schedule followed by a second dosing schedule. In an aspect, a first dosing schedule comprises a treatment or induction dose. In an aspect, a second dosing schedule comprises a maintenance dose. For example, a pharmaceutically active maintenance dose of a second dosage schedule can be lower than or equal to a pharmaceutically active induction dose of a first dosing schedule. In other examples, a maintenance dose of a second dosing schedule can be higher than an induction dose of a first dosing schedule.

At least one of a first and second dosing schedule for administering a pharmaceutical composition can comprise administration of the composition at least once daily for at least one day. In an aspect, at least one of a first or second dosing schedule comprises administration of the composition at least once daily for at least 2, 3, 4, 5, 6, 7, 8, 9, 10, 11, 12, 13, 14, or 15 consecutive days. In an aspect, at least one of a first or second dosing schedule comprises administration of the composition at least once daily for at least 1, 2, 3, 4, 5, 6, 7, 8, 9, 10, 11, or 12 consecutive weeks. In an aspect, at least one of a first or second dosing schedule comprises administration of the composition for at most 4, 5, 6, 7, 8, 9, 10, 11, 12, 13, 14, 15, 16, 17, 18, 19, or 20 consecutive days or weeks. In an aspect, at least one of a first or second dosing schedule comprises administration of the composition for at most 1, 2, 3, 4, 5, 6, 7, 8, 9, 10, 11, or 12 consecutive weeks or months. In an aspect, at least one of a first or second dosing schedule comprises administration of the composition for at least 1, 2, 3, 4, 5, 6, 7, 8, 9, 10, 11, or 12 consecutive months or years, chronically for a subject's entire life span, or an indefinite period of time.

In an aspect, at least one of a first or second dosing schedule used in a method can be once-a-week, twice-a-week, or thrice-a-week.

In an aspect, at least one of a first and second dosing schedule can last for at least about 2, 4, 6, 8, 10, 12, 18, 24, 36, 48, 72, or 96 months. In an aspect, a second dosing schedule lasts permanently, for a treated subject's entire life span, or an indefinite period of time. In an aspect, at least one of a first and second dosing schedule is a continuous dosing schedule. In an aspect, at least one of a first and second dosing schedule is an intermittent dosing schedule. In an aspect, at least one of a first and second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In an aspect, at least one of a first and second dosing schedule comprises administering a dose every other day, every two days, or every 3, 4, 5, 6, 7, 8 days. In an aspect, a dose is administered for an extended period of time with or without titration (or otherwise changing the dosage or dosing schedule).

In an aspect, the interval between a first and a second dosing schedule is at least about 1, 2, 3, 4, 5, 6, or 7 days, or at least about 1, 2, 3, 4, 5, 6, 7, 8, 9, 10, 11, or 12 weeks, or at least about 1, 2, 3, 4, 6, 7, 8, 9, 10, 11, or 12 months.

In an aspect, a second dosing schedule (e.g., a maintenance dose) comprises a dosage about 2, 3, 4, 5, 6, 7, 8, 9, 10, 20, 30, 40, 50, 75, 100, 200, 400, 800, 1000, 5000 or more fold lower than the dosage used in a first dosing schedule (e.g., an initial induction dose). In another aspect, a second dosing schedule (e.g., a maintenance dosing schedule) has an equal or lower dosing frequency than a first dosing schedule (e.g., an initial treatment dosing schedule). In an aspect, a second dosing schedule (e.g., a maintenance dosing schedule) has a higher dosing interval than a first dosing schedule (e.g., an initial treatment dosing schedule). In an aspect, all doses in an initial treatment dosing schedule are from a single donor. In another aspect, doses in an initial treatment dosing schedule are from multiple donors. In an aspect, all doses in a maintenance dosing schedule are from a single donor. In another aspect, doses in a maintenance dosing schedule are from multiple donors.

In an aspect, a first dosing schedule comprises administration of a single dose of a pharmaceutical composition to a subject. In an aspect, a second dosing schedule comprises administration of either a single dose or multiple doses of the pharmaceutical composition to the subject, wherein the dose of the pharmaceutical composition during the second dosing schedule is less than the dose of the pharmaceutical composition during the first dosing schedule.

In various aspects, methods described herein are useful in treatment of a human subject. In some aspects, the human is a pediatric human. In other aspects, the human is an adult human. In other aspects, the human is a geriatric human. In other aspects, the human may be referred to as a patient. In some aspects, the human is a female. In some aspects, the human is a male.

In certain aspects, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1-year-old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

### Additional Therapeutic Agents and Co-Formulation

The pharmaceutical compositions described herein can include one or more therapeutic agents in addition to a bacterial mixture, which can be administered to a subject in need thereof in a method described herein. The additional therapeutic agent can be administered simultaneous or sequential with a bacterial mixture (e.g., comprising one or more bacterial isolates and/or a preparation of uncultured fecal bacteria) described herein. Further, the present compositions and formulations can comprise the additional therapeutic agent (e.g. via co-formulation). For example, the additional therapeutic agent, one or more bacterial isolates, and preparation of uncultured fecal bacteria can be combined into a single formulation.

In an aspect, the additional therapeutic agent and bacterial mixture are administered to a subject simultaneously. The term “simultaneously” as used herein, means that the additional therapeutic agent and the bacterial mixture are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the bacterial mixture can be by simultaneous administration of a single formulation (e.g., a formulation comprising the additional therapeutic agent and a bacterial mixture) or of separate formulations (e.g., a first formulation including the additional therapeutic agent and a second formulation including the bacterial mixture).

Co-administration does not require an additional therapeutic agent to be administered simultaneously, if the timing of its administration is such that the pharmacological activities of the additional therapeutic agent and the bacterial mixture (e.g., comprising one or more bacterial isolates and/or a preparation of uncultured fecal bacteria) overlap in time. For example, the additional therapeutic agent and the bacterial mixture can be administered sequentially. The term “sequentially” as used herein means that the additional therapeutic agent and the bacterial mixture are administered with a time separation of more than about 60 minutes. For example, the time between the sequential administration of the additional therapeutic agent and the bacterial mixture can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent and the bacterial mixture being administered. Either of the additional therapeutic agent or the bacterial mixture can be administered first.

In a further aspect, the additional therapeutic agent and the bacterial mixture can be administered to a subject simultaneously but the release of additional therapeutic agent and the bacterial mixture from their respective dosage forms (or single unit dosage form if co-formulated) in the GI tract can occur sequentially.

Co-administration also does not require multiple additional therapeutic agents to be administered to the subject by the same route of administration as a bacterial mixture. Rather, each additional therapeutic agent can be administered by any appropriate route, for example, parenterally or non-parenterally.

In some aspects, the additional therapeutic agent is an agent used to treat one or more symptoms of ASD in a subject. In some aspects, the additional therapeutic agent is selected from the group consisting of isperidone, fluoxetine, aripiprazole, vitamin D, levocarnitine, and a combination thereof.

In some aspects, the additional therapeutic agent is an anti-inflammatory agent such as steroidal anti-inflammatory agents or non-steroidal anti-inflammatory agents (NSAIDS). Steroids, particularly the adrenal corticosteroids and their synthetic analogues, are well known in the art. Non-limiting examples of corticosteroids that can be administered to a subject as an additional therapeutic agent include hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that can be used, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, indomethacin, and a combination thereof. Additional anti-inflammatory agents are described, for example, in U.S. Pat. No. 4,537,776, the entire contents of which is hereby incorporated by reference herein in its entirety.

In some aspects, an additional therapeutic agent that can be incorporated into a pharmaceutical composition is a prebiotic. A prebiotic is a compound or compounds (e.g. comprising one or more nutrients) administered to a subject to promote the growth, proliferation, or activity of one or more microorganisms (e.g., bacteria) in the intestine of the subject (e.g., by providing a substrate to be metabolized by the one or more microorganisms). Without wishing to be bound by theory, prebiotics can be added to a pharmaceutical composition to nutritionally supplement bacteria in the endogenous microbiome of the subject and/or in the pharmaceutical composition itself, e.g., to stimulate the growth or activity of one or more strains of a preparation of uncultured fecal bacteria and/or one or more bacterial isolates. Additionally, one or more prebiotics can be added to a composition to buffer against “shock” to bacteria cells when transitioning those cells to a new environment, for example, subsequent to the isolation and/or purification of a preparation of uncultured fecal bacteria, or before or after freezing, freeze-drying, spray-drying, reconstitution in solution and the like.

Non-limiting examples of prebiotics that can be added to a pharmaceutical composition include amino acids, lactic acid, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, xylooligosaccharides (XOSs), and a combination thereof. Illustrative prebiotics include complex carbohydrates, amino acids, peptides, or other essential nutritional components for the survival of the bacterial composition.

In an aspect, a subject is not pretreated with a prebiotic prior to treatment with a pharmaceutical composition. In another aspect, the pharmaceutical composition is not supplemented with a prebiotic.

In an aspect, a prebiotic can be included (e.g., in dry or liquid forms) in a pharmaceutical composition described herein, for example, comprising a bacterial mixture.

Alternately, or additionally, a prebiotic to be administered to a subject (e.g. having one or more symptoms of ASD) can be included (e.g., in dry or liquid forms) in a distinct pharmaceutical composition lacking a bacterial mixture.

A prebiotic can be administered to a subject before, contemporaneously with, and/or after administration of a pharmaceutical composition comprising a bacterial mixture, either in the same pharmaceutical composition or in a separate pharmaceutical composition.

A prebiotic can be provided and administered in a single dose or in multiple doses. When provided as a single dose, a single composition can comprise only one prebiotic or a mixture of prebiotics. When provided in multiple doses, each composition dosed to the subject can comprise a single prebiotic or a mixture of prebiotics, and/or a first composition dosed to the subject can comprise a different prebiotic or prebiotics than a second composition dosed to the subject.

As examples, when multiple doses are provided, a first composition comprising a prebiotic can include a first prebiotic, e.g., inulin, and a second composition can include a different prebiotic, e.g., pectin, with or without the first prebiotic. Alternately, a first composition can include a combination of prebiotics, e.g., inulin and pectin and a second composition can include a different combination of prebiotics, e.g., inulin and a FOS. A first composition can include a combination of prebiotics and a second composition can include only one prebiotic.

The amount of prebiotic included in a composition depends on the specific prebiotic, the specific bacterial strain or strains targeted by the prebiotic, and/or the disease state of the subject/patient.

In some aspects, an additional therapeutic agent that can be incorporated into a pharmaceutical composition is an antidiarrheal agent. Non-limiting examples of antidiarrheal agents suitable for inclusion in a pharmaceutical composition described herein include, but are not limited to, DPP-IV inhibitors, natural opioids, such as tincture of opium, paregoric, and codeine, synthetic opioids, such as diphenoxylate, difenoxin and loperamide, bismuth subsalicylate, lanreotide, vapreotide and octreotide, motiln antagonists, COX2 inhibitors like celecoxib, glutamine, thalidomide and traditional antidiarrheal remedies, such as kaolin, pectin, berberine and muscarinic agents, and a combination thereof.

In some aspects, the additional therapeutic agent incorporated into a pharmaceutical composition can be an analgesic. Analgesics useful in the compositions and methods described herein include, without limitation, morphine, codeine, heroine, methadone and related compounds, thebaine, orpiavine, and their derivatives, buprenorphine, the piperidines, morphinans, benzomorphans, tetrahydroisoquinolines, thiambutanes, benzylamines, tilidine, viminol, nefopam, capsaicin(8-methyl-N-vanillyl-6E-nonenamide), “synthetic” capsaicin(N-vanillylnonamide) and related compounds, and a combination thereof.

In some aspects, the additional therapeutic agent is an anti-bacterial agent, which includes, but is not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); carbapenem antibiotics (ertapenem, doripenem, imipenem/cilastatin, and meropenem); and a combination thereof. In some aspects, the anti-bacterial agent can be any of the penicillin, cephalosporin, monobactam, and carbapenem antibiotics, or a combination thereof.

In one aspect, a method further comprises pretreating a subject with an antibiotic composition prior to administering a therapeutic bacterial mixture. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In another aspect, a subject is not pretreated with an antibiotic composition prior to administering a bacterial mixture. In another aspect, the pharmaceutical composition is not supplemented with an antibiotic composition. In a further aspect, a method further comprises pretreating a subject with an anti-inflammatory drug prior to administration of a bacterial mixture. In yet another aspect, a subject is not pretreated with an anti-inflammatory drug prior to administering a bacterial or mixture. In another aspect, a bacterial mixture is not supplemented with an anti-inflammatory.

Delivery of an additional therapeutic agent can be targeted to various parts of the GI tract, as described herein.

The pharmaceutical compositions described herein (e.g. comprising one or more bacterial mixtures comprising for example one or more bacterial isolates and/or a preparation of uncultured fecal bacteria) can be administered to a subject in need thereof for the treatment or prevention of one or more disorders, diseases, or conditions. In an aspect, a pharmaceutical composition is administered to a subject to prevent or treat one or more symptoms of ASD in the subject. Provided herein is a method of treating or preventing one or more symptoms of ASD in a subject in need thereof, the method comprising administering to the subject a pharmaceutical composition described herein.

The methods provided herein result in, or are aimed at achieving, a detectable improvement in one or more indicators or symptoms of ASD including, but not limited to, changes in eye tracking, skin conductance and/or EEG measurements in response to visual stimuli, difficulties engaging in and responding to social interaction, verbal and nonverbal communication problems, repetitive behaviors, intellectual disability, difficulties in motor coordination, attention issues, sleep disturbances, and physical health issues such as gastrointestinal disturbances.

Several screening instruments are known in the art for evaluating a subject's social and communicative development and thus can be used as aids in screening for and detecting changes in the severity of impairment in communication skills, social interactions, and restricted, repetitive and stereotyped patterns of behavior characteristic of autism spectrum disorder. Evaluation can include neurologic and genetic assessment, along with in-depth cognitive and language testing. Additional measures developed specifically for diagnosing and assessing autism include the Autism Diagnosis Interview-Revised (ADI-R), the Autism Diagnostic Observation Schedule (ADOS-G) and the Childhood Autism Rating Scale (CARS).

Autism Diagnostic Interview-Revised (ADI-R) is a 2-hour structured interview and is one of the primary tools used for clinical diagnosis of autism and autism spectrum disorders. As used herein, the ADI-R can be used to verify the diagnosis of ASD. The ADI-R is a standardized, semi-structured clinical review for caregivers of children and adults. The interview contains 93 items and focuses on behaviors in three content areas or domains: quality of social interaction (e.g., emotional sharing, offering and seeking comfort, social smiling and responding to other children); communication and language (e.g., stereotyped utterances, pronoun reversal, social usage of language); and repetitive, restricted and stereotyped interests and behavior (e.g., unusual preoccupations, hand and finger mannerisms, unusual sensory interests). The measure also includes other items relevant for treatment planning, such as self-injury and over-activity. Responses are scored by the clinician based on the caregiver's description of the child's behavior. The ADI-R interview generates scores in each of the three content areas (i.e., communication and language, social interaction, and restricted, repetitive behaviors). Elevated scores indicate problematic behavior in a particular area. Scores are based on the clinician's judgment following the caregiver's report of the child's behavior and development. For each item, the clinician gives a score ranging from 0 to 3. A score of 0 indicates that behavior of the type specified in the coding is not present; a score of 1 indicates that behavior of the type specified is present in an abnormal form, but not sufficiently severe or frequent to meet the criteria for a 2; a score of 2 indicates that definite abnormal behavior meeting the criteria specified; and a score of 3 indicates extreme severity of the specified behavior. The authors of the measure recode 3 as a 2 in computing the algorithm. There are also scores of 7 (indicating definite abnormality in the general area of the coding, but not of the type specified), 8 (indicating not applicable), and 9 (indicating not known or not asked) given under certain circumstances, which all are converted to 0 in computing the algorithm.

A classification of autism is given when scores in all three content areas of communication, social interaction, and patterns of behavior meet or exceed the specified cutoffs, and onset of the disorder is evident by 36 months of age. The same algorithm is used for children from mental ages 18 months through adulthood, with three versions containing minor modifications: 1) a life-time version; 2) a version based on current behavior; and 3) a version for use with children under the age of 4 years. The algorithm specifies a minimum score in each area to yield a diagnosis of autism as described in ICD-10 (10^{th} version of the international Statistical Classification of Diseases and Related Health Problems) and DSM-IV (Diagnostic and Statistical Manual of Mental Disorders, 4th Edition). The total cutoff score for the communication and language domain is 8 for verbal subjects and 7 for nonverbal subjects. For all subjects, the cutoff for the social interaction domain is 10, and the cutoff for restricted and repetitive behaviors is 3.

According to CARS, evaluators rate the subject on a scale from 1 to 4 in each of 15 areas: Relating to People; Imitation; Emotional Response; Body Use; Object Use; Adaptation to Change; Visual Response; Listening Response; Taste, Smell, and Touch Response and Use; Fear; Verbal Communication; Nonverbal Communication; Activity; Level and Consistency of Intellectual Response; and General Impressions.

A second edition of CARS, known as the Childhood Autism Rating Scale—2 or CARS-2, was developed by Schopler et al. (Childhood Autism Rating Scale—Second edition (CARS2): Manual. Los Angeles: Western Psychological Services, 2010). The original CARS was developed primarily with individuals with co-morbid intellectual functioning and was criticized for not accurately identifying higher functioning individuals with ASD. CARS-2 retained the original CARS form for use with younger or lower functioning individuals (now renamed the CARS2-ST for “Standard Form”), but also includes a separate rating scale for use with higher functioning individuals (named the CARS2-HF for “High Functioning”) and an unscored information-gathering scale (“Questionnaire for Parents or Caregivers” or CARS2-QPC) that has utility for making CARS2ST and CARS2-HF ratings.

Another symptom rating instrument useful for assessing changes in symptom severity before, during, or following treatment according to a method provided herein is the Aberrant Behavior Checklist (ABC). See Aman et al., Psychometric characteristics of the aberrant behavior checklist. Am J Ment Defic. 1985 March; 89(5):492-502. The ABC is a symptom checklist for assessing problem behaviors of children and adults with developmental disabilities (intellectual disability, ASD, cerebral palsy, epilepsy). It is also useful for classifying problem behaviors of children and adolescents with developmental disabilities at home, in educational settings, community-based facilities, and in developmental centers. It contains 58 items that resolve onto 5 subscales and administration time is 10-15 minutes.

The ABC identifies the setting where the person was observed and the presence/severity of functional limitations. Specific symptoms are rated and an extensive manual provides comprehensive descriptions for each assessed behavior. The ABC five subscales include: (1) irritability/agitation, (2) lethargy/social withdrawal, (3) stereotypic behavior, (4) hyperactivity/noncompliance, and (5) inappropriate speech. ABC-2 is the revised version of ABC. ABC-2 also contains 58 items resolved into the same 5 subscales as ABC.

Another symptom rating instrument useful for assessing changes in symptom severity before, during, or following treatment according to a method provided herein is the Social Responsiveness Scale (SRS). The SRS scale is a 65-item scale that assesses social impairments, a core issue in autism, including social awareness, social information processing, capacity for reciprocal social communication, social anxiety/avoidance, and autistic preoccupations and traits. See Constantino et al., Validation of a brief quantitative measure of autistic traits: comparison of the social responsiveness scale with the autism diagnostic interview-revised. J Autism Dev Disord. 2003 August; 33(4):427-33.

As used herein, SRS-2 is an updated edition of SRS. SRS-2 is also a 65-item scale that assesses social impairments, a core issue in autism, including social awareness, social information processing, capacity for reciprocal social communication, social anxiety/avoidance, and autistic preoccupations and traits.

As used herein, the Vineland Adaptive Behavior Scale II (VABS-II) is a measure of the functioning level in four different domains: Communication, Daily Living Skills, Socialization, and Motor Skills, and 11 sub-domains. The raw scores are converted into an age equivalent score. It complements the ABC, which assesses problem behaviors. See Sara et al., Vineland Adaptive Behavior Scales, Second Edition (Vineland™-II), Pearson Publishing, 2005. The ranges for VABS-II include 0-80 borderline adaptive functioning; 51-70: mildly deficient adaptive functioning; 35-50: moderately deficient adaptive behavior; 20-35: severely deficient adaptive behavior; less than 20: markedly or profoundly deficient adaptive behavior. Scores above 80 are classified in approximately the same ranges (low average, average, above average, superior) as IQ scores.

As used herein, the VABS version III (VABS-III) is an individually administered measure (scale) of adaptive behavior used in the assessment of individuals with intellectual, developmental and other disabilities. The scale measures adaptive behavior in four major domains: communication, daily living skills, socialization, and gross motor Skills. The first three of these domains include several subdomains (communication receptive communication, expressive communication, written communication, daily living skills domestic skills, personal skills, community skills, socialization interpersonal relationships, play/leisure, and coping Skills). VABS-III is parent administered online through Q-global (a secure online testing platform) and takes approximately 40 minutes to complete. The parent does not need to complete the tool all at one time, and may start and stop the tool as needed until completed. All scoring is completed online and available to the Sponsor upon completion of the tool. The completion of the measure may be made up to 48 hours prior to the visit but no later than the day of the visit.

As used herein, Parent Global Impressions-III (PGI-III) is an expanded version of the PGI-R. See Adams et al., Effect of a Vitamin/Mineral Supplement on Children with Autism, BMC Pediatrics, 11:111(2011). The PGI-III evaluates changes in 17 areas, and overall, using a 7-point scale ranging from “much worse” to “much better”. An “average change” is computed by computing the average in all 18 scores of the PGI-III-Final. PGI-III is preferred because it is found to be more reliable to ask parents directly about observed changes than to have them estimate symptom severity at beginning and end and then compute a difference. Also, the use of a 7-point scale to detect changes seems to yield a high sensitivity to changes.

As used herein, another symptom rating instrument is Clinical Global Impressions Scale (CGI), which is a 3-item scale that measures illness severity as well as treatment response in subjects with psychiatric symptoms. The rating is performed by the clinician or a trained rater. Following initial assessment (CGI-Severity, or CGI-S), consecutive assessments are performed to determine response to an intervention. CGI-Improvement (CGI-I) measures severity of illness, clinical progress and therapeutic ‘efficacy’ (the latter includes assessment of treatment related AE as well as efficacy).

The CGI-S scale is a seven-point scale rating that assesses the patient's mental illness as follows: 1=normal, not at all ill; 2=borderline mentally ill; 3=mildly ill; 4=moderately ill; 5=markedly ill; 6=severely ill; 7=among the most extremely ill patients. See Busner et al., The Clinical Global Impressions Scale, Psychiatry, 4(7): 28-37, 2007, incorporated herein by reference in its entirety. (“Busner 2007”)

The CGI-I scale is also a seven-point rating scale that compares the patient's overall clinical condition to the one week period just prior to the initiation of medication use (or baseline visit). The patient's condition is rated in comparison to prior to medication initiation as follows: 1=very much improved since the initiation of treatment; 2=much improved; 3=minimally improved; 4=no change from baseline (the initiation of treatment); 5=minimally worse; 6=much worse; 7=very much worse since the initiation of treatment. See Busner 2007.

Efficacy Index is measured on a 4-point scale (ranging from ‘unchanged or worse and side effects outweigh therapeutic effects’ to ‘marked improvement and no side-effects’). At baseline screening, only the CGI-S is evaluated. At subsequent visits, Global improvement and Efficacy Index are measured (CGI-I). At the last visit (for example week 32), all three measures should be performed.

The specific parameters to be assessed in the CGI (i.e. with regard to severity, improvement and treatment impact) should derive from the anchor points or 1-3 key behaviors identified in the initial CY-BOCS.

Children's Yale Brown Obsessive Compulsive Scale (CY-BOCS) is a scale designed to rate the severity of obsessive and compulsive disorder in adolescents, ages 6 to 17 years. CY-BOCS is utilized to diagnose obsessive compulsive disorder, ascertain current and past symptoms, and to assess the severity of obsessive compulsive disorder over a period of time. The CY-BOCS score ranges in severity as follows: 0-7=subclinical, 8-15=mild, 16-23=moderate, 24-31=severe, and 32-40=extreme. See McKay et al., The Children's Yale-Brown Obsessive-Compulsive Scale: item structure in an outpatient setting, Psychol Assess, 15(4): 578-81, 2003, hereby incorporated herein by reference in its entirety. As used herein, the CY-BOCS provides the clinician with anchor points or 1-3 key behaviors which the clinician will track over time using the CGI-I scoring system.

The present inventors have found that restoring the species diversity of gut bacteria helps to treat autistic symptoms in patients in need thereof. In one aspect, this application provides a method for treating one or more symptoms of an autism spectrum disorder (ASD) in a subject in need thereof, the method comprising administering to the subject an amount of a pharmaceutical composition effective for treating the ASD, where the pharmaceutical composition comprises a preparation of uncultured fecal bacteria and at least one bacterial isolate, where the subject exhibits at least a 10% reduction in ASD symptom severity after the treatment as compared to before initiating the treatment. In one aspect, ASD symptom severity is assessed by Childhood Autism Rating Scale (CARS). In another aspect, ASD symptom severity is assessed by Childhood Autism Rating Scale 2—Standard Form (CARS2-ST). In a further aspect, ASD symptom severity is assessed by Childhood Autism Rating Scale 2—High Functioning (CARS2-HF). In one aspect, ASD symptom severity is assessed by Aberrant Behavior Checklist (ABC). In another aspect, ASD symptom severity is assessed by Social Responsiveness Scale (SRS). In another aspect, ASD symptom severity is assessed by Vineland Adaptive Behavior Scale II (VABS-II). In another aspect, ASD symptom severity is assessed by CGI. In another aspect, ASD symptom severity is assessed by CGI-S. In another aspect, ASD symptom severity is assessed by CY-BOCS. In another aspect, ASD symptom severity is assessed by ADI-R. In one aspect, a treatment results in an improvement of at least 10%, 20%, 30%, 40%, 50%, 60%, 70%, 80%, or 90% based on the Leiter International Performance Scale (see Roid, G. H., & Miller, L. J. (1997). Leiter International Performance Scale—Revised. Wood Dale, Ill.: Stoelting) in an ASD patient. In another aspect, a Leiter score improvement is measured after at least 8, 16, 24, 32, 40, 50, 60, or 80 weeks of treatment and compared to a Leiter score prior to the treatment.

One of ordinary skill in the art understands that the foregoing assessment systems are only exemplary tools for evaluating ASD-related social and cognitive symptoms. Other similar tools can be used or designed to evaluate core ASD-related symptoms. For example, in one aspect, a treatment results in an improvement of at least 10%, 20%, 30%, 40%, 50%, 60%, 70%, 80%, or 90% based on Autism Treatment Evaluation Checklist (ATEC). See Rimland and Edelson: Autism Treatment Evaluation Checklist: Statistical Analyses. Autism Research Institute 2000. In another aspect, a treatment results in an improvement of at least 10%, 20%, 30%, 40%, 50%, 60%, 70%, 80%, or 90% based on Pervasive Developmental Disorders Behavior Inventory (PDD-BI). See Cohen et al., The PDD Behavior Inventory: a rating scale for assessing response to intervention in children with pervasive developmental disorder. J Autism Dev Disord. 2003 33(1):31-45. In yet another aspect, a treatment results in an improvement of at least 10%, 20%, 30%, 40%, 50%, 60%, 70%, 80%, or 90% based on Severity of Autism Scale (SAS). See Adams et al., The severity of autism is associated with toxic metal body burden and red blood cell glutathione levels. J Toxicol. 2009, 2009:532640. In a further aspect, an improvement of autism-related symptoms or an symptom severity reduction is assessed based on any one of the system or scale mentioned in Aman et al., Outcome Measures for Clinical Drug Trials in Autism, CNS Spectr. 9(1): 36-47 (2004). In a further aspect, an improvement of autism-related symptoms or an symptom severity reduction is assessed based on any one of the symptom characterization systems listed in Table 1. In one aspect, an symptom improvement over any one of the foregoing systems is measured after at least 8, 16, 24, 32, 40, 50, 60, or 80 weeks of treatment and compared to a Leiter score prior to the treatment. In one aspect, an symptom improvement over any one of the foregoing systems is measured after discontinuing treatment for at least 2, 4, 6, 8, 10 or more weeks and compared to a measurement prior to the treatment.

TABLE 1
Selected outcome measures that can be used to monitor
core ASD-related social and cognitive symptoms.
Validated Outcome Measures

| Tool | Description | Rater |
|---|---|---|
| | Autism Symptoms | |
| ADOS | The Autism Diagnostic Observation Schedule (ADOS) is a gold standards | Trained Examiner |
| | instrument for diagnosing ASD with the largest evidence base and highest | |
| | sensitivity and specificity. | |
| OACIS | The Ohio Autism Clinical Impression Scale was developed to be sensitive to | Clinician |
| | subtle, but clinically-meaningful changes in core and associated ASD symptoms | |
| | using a focused scaling system that assesses severity and improvement in ASD | |
| | behaviors similar to the widely used Clinical Global Impression Scale. | |
| SRS | The Social Responsiveness Scale is a standardized and validated quantitative | Parent or Teacher |
| | scale that measures the severity and type of social impairments that are | |
| | characteristic of ASD. | |
| SCQ | Social Communication Questionnaire is brief instrument that evaluates | Parent or Teacher |
| | communication skills and social functioning. Both the current and lifetime | |
| | editions will be used as appropriate. | |
| AIM | The Autism Impact Measure is a recently developed parent-report measure that | Parent |
| | assesses both frequency and impact of current core ASD symptoms during the past | |
| | 2-weeks. Initial studies have demonstrated excellent psychometric properties and | |
| | construct validity. | |
| CGI | The Clinical Global Impressions is a validated questionnaire which serves as a | Clinician |
| | stand-alone assessment of the clinician's view of the patient's global functioning | |
| | prior to and after initiating a study medication. CGI has been shown to correlate | |
| | well with standard, well-known research drug efficacy scales (e.g., Positive and | |
| | Negative Syndrome Scale, Scale for the Assessment of Negative Symptoms, and | |
| | others). | |
| CY-BOCS | The Children's Yale-Brown Obsessive-Compulsive Scale is one of the most | Clinician or |
| | popular measures of symptom severity for obsessive-compulsive disorder. The | Trained Examiner |
| | scale includes a symptom checklist, target symptom list, and a severity rating. | |
| | Behavior | |
| ABC | The Aberrant Behavior Checklist is a validated questionnaire that rates symptoms | Parent or Teacher |
| | of hyperactivity, irritability, lethargy, and stereotypic behavior in individuals with | |
| | developmental disabilities. It has been used in multiple clinical trials in ASD and | |
| | has convergent and divergent validity. | |
| CBCL | Child Behavior Checklist is an easy to complete standardized questionnaire that | Parent or Teacher |
| | assesses a wide range of behaviors associated with ASD symptoms, including | |
| | anxiety, depression, withdraw, sleep problems, somatic problems, and aggressive | |
| | and destructive behavior. | |
| BASC | The Behavioral Assessment System for Children provides scales of cognition | Parent or Teacher |
| | function, behavior, social function, and academic problems. This scale measures a | |
| | wide range of behaviors including hyperactivity, attention, depression, anxiety, | |
| | and executive function. | |
| | Language | |
| CELF | The Clinical Evaluations of Language Fundamentals is one of the only | Trained Examiner |
| | standardized, well-validated language assessment instruments that spans the age | |
| | range of most participants (using both CELF-preschool-2 and CELF-4). It | |
| | assesses a wide range of language skills that are only partially measured by other | |
| | language tests, including high-level language skills that are abnormal in | |
| | individuals with ASD, such as language pragmatics and has been used in several | |
| | recent studies focusing on core language deficits in ASD. | |
| PLS | The Preschool Language Scale-4 is used in conjunction with the CELF since it is | Trained Examiner |
| | also a standardized, well-validated language assessment instrument and can | |
| | measure subtle changes in language in children with poor language abilities. | |
| | Adaptive Behavior | |
| VABS | The Vineland Adaptive Behavior Scale is a widely used standardized, well- | Trained |
| | validated assessment tool for children with developmental delays that measures | Interviewer |
| | functional abilities within several domains. It is particularly useful for children | |
| | with intellectual disability which commonly co-occurs with ASD and has valid | |
| | measures of social impairments in children with ASD. | |
| | Intellect | |
| RIAS-NV | RIAS is a comprehensive intelligence test, for 3-94 years of age range, that | Clinician |
| | provides the necessary information to help clinicians make decisions regarding | |
| | classification, selection, and educational placement. Nonverbal intelligence is | |
| | assessed by measuring reasoning and spatial ability using novel situations and | |
| | stimuli that are predominantly nonverbal. | |
| Leiter-R | The Leiter-R, due to its non-verbal nature, is an excellent unbiased measure of | Trained Examiner |
| | intellect when language impairment exists. It assesses a wide range of ages (2-21 | |
| | years) and contains attention and memory batteries which are skills often | |
| | disrupted in ASD. The Leiter-R is designed to measure growth in all domains it | |
| | assesses, making it sensitive to change due to treatment. Studies have shown good | |
| | psychometric properties and verified that it is generally recommended for use in | |
| | children with ASD. | |
| WISC | The Wechsler Intelligence Scale for Children is one of the oldest and most widely | Trained Examiner |
| WPPSI | used tests of intelligence for children. For children younger than 6 years the | |
| | Wechsler Preschool and Primary Scale of Intelligence test is used. One | |
| | disadvantage when using this with children with ASD is its reliance on language. | |

In one aspect, a treatment with a pharmaceutical composition described herein achieves at least 10%, 20%, 30%, 40%, 50%, 60%, 70%, 80%, or 90% reduction in ASD symptom severity after 2 or more weeks of treatment as compared to before initiating the treatment, where the ASD symptom severity is assessed by a method selected from the group consisting of CARS, CARS2-ST, CARS2-HF, ABC, SRS, and VABS-II. In one aspect, a treatment achieves at least 10%, 20%, 30%, 40%, 50%, 60%, 70%, 80%, or 90% reduction in ASD symptom severity after 4 or more weeks of treatment as compared to before initiating the treatment, where the ASD symptom severity is assessed by a method selected from the group consisting of Childhood Autism Rating Scales-2 (CARS-2), Aberrant Behavior Checklist-2 (ABC-2), Reynolds Intellectual Assessment Scales—Nonverbal (RIAS-NV), Social Responsiveness Scale-2 (SRS-2), and Parent Global Impression-III (PGI-III). In one aspect, a treatment achieves at least 10%, 20%, 30%, 40%, 50%, 60%, 70%, 80%, or 90% reduction in ASD symptom severity after 6 or more weeks of treatment as compared to before initiating the treatment, where the ASD symptom severity is assessed by a method selected from the group consisting of Childhood Autism Rating Scales-2 (CARS-2), Aberrant Behavior Checklist-2 (ABC-2), Reynolds Intellectual Assessment Scales—Nonverbal (RIAS-NV), Social Responsiveness Scale-2 (SRS-2), and Parent Global Impression-III (PGI-III). In one aspect, a treatment achieves at least 10%, 20%, 30%, 40%, 50%, 60%, 70%, 80%, or 90% reduction in ASD symptom severity after 2 or more weeks of treatment as compared to before initiating the treatment, where the ASD symptom severity is assessed by a method selected from the group consisting of Childhood Autism Rating Scales-2 (CARS-2), Aberrant Behavior Checklist-2 (ABC-2), Reynolds Intellectual Assessment Scales—Nonverbal (RIAS-NV), Social Responsiveness Scale-2 (SRS-2), and Parent Global Impression-III (PGI-III).

In another aspect, a treatment with a pharmaceutical composition described herein achieves between 10% and 20%, between 10% and 30%, between 10% and 40%, between 10% and 50%, between 10% and 60%, between 10% and 70%, between 10% and 80%, between 10% and 90%, between 20% and 30%, between 20% and 40%, between 20% and 50%, between 20% and 60%, between 20% and 70%, between 20% and 80%, between 20% and 90%, between 30% and 40%, between 30% and 50%, between 30% and 60%, between 30% and 70%, between 30% and 80%, between 30% and 90%, between 40% and 50%, between 40% and 60%, between 40% and 70%, between 40% and 80%, between 40% and 90%, between 50% and 60%, between 50% and 70%, between 50% and 80%, or between 50% and 90% reduction in ASD symptom severity after 2 or more weeks of treatment as compared to before initiating the treatment, where the ASD symptom severity is assessed by a method selected from the group consisting of CARS-2, ABC-2, RIAS-NV, SRS-2, and PGI-III. In another aspect, a treatment achieves between 10% and 90%, between 20% and 80%, between 30% and 70%, or between 40% and 60% reduction in ASD symptom severity after 2 or more weeks of treatment as compared to before initiating the treatment, where the ASD symptom severity is assessed by a method selected from the group consisting of CARS-2, ABC-2, RIAS-NV, SRS-2, and PGI-III. In another aspect, a treatment achieves between 10% and 90%, between 20% and 80%, between 30% and 70%, or between 40% and 60% reduction in ASD symptom severity after 12 or more weeks of treatment as compared to before initiating the treatment, where the ASD symptom severity is assessed by a method selected from the group consisting of CARS-2, ABC-2, RIAS-NV, SRS-2, and PGI-III. In another aspect, a treatment achieves between 10% and 90%, between 20% and 80%, between 30% and 70%, or between 40% and 60% reduction in ASD symptom severity after 2 or more weeks of treatment as compared to before initiating the treatment, where the ASD symptom severity is assessed by a method selected from the group consisting of CARS-2, ABC-2, RIAS-NV, SRS-2, and PGI-III. In another aspect, a treatment achieves between 10% and 90%, between 20% and 80%, between 30% and 70%, or between 40% and 60% reduction in ASD symptom severity after 24 or more weeks of treatment as compared to before initiating the treatment, where the ASD symptom severity is assessed by a method selected from the group consisting of CARS-2, ABC-2, RIAS-NV, SRS-2, and PGI-III.

In one aspect, a treatment with a pharmaceutical composition described herein achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by CARS. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by CARS2-ST. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by CARS2-HF. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by ABC. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by SRS. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by VABS-II.

In one aspect, an ASD subject being treated exhibits no gastrointestinal (GI) symptom prior to initiating a treatment. In one aspect, an ASD subject being treated exhibits no gastrointestinal (GI) symptom for at least 1 day, 2 days, 3 days, 4 days, 5 days, 6 days, 1 week, 2 weeks, 3 weeks, 4 weeks, 1 month, 2 months, 6 months, 1 year or 2 years prior to treatment. In another aspect, an ASD subject being treated exhibits one or more GI symptoms prior to initiating a treatment. In one aspect, an ASD subject being treated exhibits a gastrointestinal (GI) symptom on a continuous or intermittent basis for at least 1 day, 2 days, 3 days, 4 days, 5 days, 6 days, 1 week, 2 weeks, 3 weeks, 4 weeks, 1 month, 2 months, 6 months, 1 year or 2 years prior to treatment. In one aspect, an ASD subject being treated exhibits symptoms of chronic abnormal bowel function for a minimum of 1 year. Such symptoms of chronic abnormal bowel function can include, for example, constipation and/or diarrhea. In one aspect, an ASD subject being treated exhibits at least a 20%, 30%, 40%, 50%, 60%, 70%, 80%, or 90% reduction in GI symptom severity after a treatment as compared to before initiating the treatment. In one aspect, GI symptom severity is assessed by the Gastrointestinal Symptom Rating Scale (GSRS). In another aspect, the GI symptom severity is assessed by the Gastrointestinal Stool and Symptom Questionnaire for Autism (GSSQA). In another aspect, a treatment achieves between 20% and 30%, between 20% and 40%, between 20% and 50%, between 20% and 60%, between 20% and 70%, between 20% and 80%, between 20% and 90%, between 30% and 40%, between 30% and 50%, between 30% and 60%, between 30% and 70%, between 30% and 80%, between 30% and 90%, between 40% and 50%, between 40% and 60%, between 40% and 70%, between 40% and 80%, between 40% and 90%, between 50% and 60%, between 50% and 70%, between 50% and 80%, or between 50% and 90% reduction in GI symptom severity in an ASD patient after 2 or more weeks of treatment as compared to before initiating the treatment, where the GI symptom severity is assessed by GSRS or GSSQA.

The GSRS is a disease-specific instrument of 15 items combined into five symptom clusters depicting Reflux, Abdominal pain, Indigestion, Diarrhea and Constipation. See Svedlund et al., Dig. Dis. Sci., 33(2):129-34(1988). The GSRS has a seven-point graded Likert-type scale where 0 represents absence of troublesome symptoms and 3 represents an extreme degree of the symptoms with half-steps to increase the sensitivity of the scales. In one aspect, a treatment method provided here reduces, alleviates, or eliminates one or more, two or more, three or more, four or more, five or more, six or more, or seven or more GI symptoms selected from the group consisting of epigastric pain, colicky abdominal pain, dull abdominal pain, undefined abdominal pain, heartburn, acid regurgitation, sucking sensations in the epigastrium, nausea and vomiting, borborygmus, abdominal distension, eructation, increased flatus, decreased passage of stools, increased passage of stools, loose stool, hard stools, urgent need for defecation, feeling of incomplete evacuation. In another aspect, a treatment method provided here reduces, alleviates, or eliminates between 2 and 4, between 4 and 6, between 6 and 8, between 8 and 10, between 10 and 12, between 2 and 3, between 2 and 4, between 2 and 5, between 2 and 6, between 2 and 7, between 2 and 8, between 2 and 9, between 2 and 10, between 2 and 11, between 2 and 12, between 3 and 4, between 3 and 5, between 3 and 6, between 3 and 7, between 3 and 8, between 3 and 9, between 3 and 10, between 3 and 11, between 3 and 12, between 4 and 5, between 4 and 6, between 4 and 7, between 4 and 8, between 4 and 9, between 4 and 10, between 4 and 11, between 4 and 12, between 5 and 6, between 5 and 7, between 5 and 8, between 5 and 9, between 5 and 10, between 5 and 11, between 5 and 12, between 6 and 7, between 6 and 8, between 6 and 9, between 6 and 10, between 6 and 11, between 6 and 12, between 7 and 8, between 7 and 9, between 7 and 10, between 7 and 11, or between 7 and 12 GI symptoms selected from the group consisting of epigastric pain, colicky abdominal pain, dull abdominal pain, undefined abdominal pain, heartburn, acid regurgitation, sucking sensations in the epigastrium, nausea and vomiting, borborygmus, abdominal distension, eructation, increased flatus, decreased passage of stools, increased passage of stools, loose stool, hard stools, urgent need for defecation, feeling of incomplete evacuation and less than 3 complete spontaneous bowel movements per week.

Gastrointestinal Stool and Symptom Questionnaire for Autism (GSSQA) is a clinical assessment tool for GI symptoms in individuals 4 to 17 years old with autism. GSSQA utilizes an Observer-Reported Outcome (ObsRO).

In one aspect, a treated subject's abdominal pain decreases from a more severe level to a less severe level, where the pain levels are selected from the group consisting of severe or crippling pains with impact on all social activities, prolonged and troublesome aches and pains causing requests for relief and interfering with many social activities, occasional aches and pains interfering with some social activities, and no or transient pain.

In another aspect, a treated subject's heartburn decreases from a more severe level to a less severe level, where the pain levels are selected from the group consisting of continuous discomfort with only transient relief by antacids, frequent episodes of prolonged discomfort; requests for relief, occasional discomfort of short duration, and no or transient heartburn.

In another aspect, a treated subject's acid regurgitation condition improves from a more severe level to a less severe level, where the condition levels are selected from the group consisting of regurgitation several times a day; only transient and insignificant relief by antacids, regurgitation once or twice a day; requests for relief, occasional troublesome regurgitation, and no or transient regurgitation.

In another aspect, a treated subject's sucking sensations in the epigastrium improves from a more severe level to a less severe level, where the condition levels are selected from the group consisting of continuous discomfort; frequent requests for food or antacids between meals, frequent episodes of prolonged discomfort, requests for food and antacids between meals, occasional discomfort of short duration; no requests for food or antacids between meals, and no or transient sucking sensation. As used herein, sucking sensation in the epigastrium represents a sucking sensation in the epigastrium with relief by food or antacids. If food or antacids are not available, the sucking sensations progress to ache, and pains.

In another aspect, a treated subject's nausea or vomiting condition improves from a more severe level to a less severe level, where the condition levels are selected from the group consisting of continuous nausea coupled with frequent vomiting, frequent and prolonged nausea with no vomiting, occasional nausea episodes of short duration, and no nausea.

In another aspect, a treated subject's borborygmus condition improves from a more severe level to a less severe level, where the condition levels are selected from the group consisting of continuous borborygmus severely interfering with social performance, frequent and prolonged episodes which can be mastered by moving without impairing social performance, occasional troublesome borborygmus of short duration, and no or transient borborygmus.

In another aspect, a treated subject's abdominal distension condition improves from a more severe level to a less severe level, where the condition levels are selected from the group consisting of continuous discomfort seriously interfering with social performance, frequent and prolonged episodes which can be mastered by adjusting the clothing, occasional discomfort of short duration, and no or transient distension.

In another aspect, a treated subject's eructation condition improves from a more severe level to a less severe level, where the condition levels are selected from the group consisting of frequent episodes seriously interfering with social performance, frequent episodes interfering with some social activities, occasional troublesome eructation, and no or transient eructation.

In another aspect, a treated subject's increased flatus condition improves from a more severe level to a less severe level, where the condition levels are selected from the group consisting of frequent episodes seriously interfering with social performance, frequent and prolonged episodes interfering with some social activities, occasional discomfort of short duration, and no increase in flatus.

In another aspect, a treated subject's decreased stool frequency improves from a more severe level to a less severe level, where the levels are selected from the group consisting of every seventh day or less frequently, every sixth day, every fifth day, every fourth day, every third day, every second day, and once a day.

In another aspect, a treated subject's increased stool frequency improves from a more severe level to a less severe level, where the levels are selected from the group consisting of seven times a day or more frequently, six times a day, five times a day, four times a day, three times a day, twice a day, and once a day.

In another aspect, a treated subject's loose-stool condition improves from a more severe level to a less severe level, where the levels are selected from the group consisting of watery, runny, somewhat loose, and normal consistency.

In another aspect, a treated subject's hard-stool condition improves from a more severe level to a less severe level, where the levels are selected from the group consisting of hard and fragmented with occasional diarrhea, hard, somewhat hard, and normal consistency. In an aspect, a treated subject's stool is evaluated using the Daily Stool Records (DSR). In one aspect, a treated subject exhibits a reduction in all of type 1 hard stool, type 2 hard stool, type 6 soft stool, type 7 liquid stool, and abnormal stool according to the DSR.

In another aspect, a treated subject's urgency for defecation improves from a more severe level to a less severe level, where the levels are selected from the group consisting of inability to control defecation, frequent feelings of urgent need for defecation with sudden need for a toilet interfering with social performance, occasional feelings of urgent need for defecation, and normal control of defecation.

In another aspect, a treated subject's feeling of incomplete evacuation improves from a more severe level to a less severe level, where the levels are selected from the group consisting of defecation extremely difficult with regular feelings of incomplete evacuation, defecation definitely difficult with often feelings of incomplete evacuation, defecation somewhat difficult; occasional feelings of incomplete evacuation, and feeling of complete evacuation without straining.

In another aspect, a treated subject's number of complete spontaneous bowel movement (CSBM) per week increases compared to baseline. In another aspect, a treated subject's CSBM increases by at least 1, 2, 3, 4, 5, 6, 7, 8, 9, or 10 complete spontaneous bowel movements (CSBM) per week after at least 1, 2, 3, 4, 5, 6, 7, or 8 weeks of treatment. In another aspect, the subject's number of complete spontaneous bowel movement (CSBM) increases by at least 1, 2, 3, 4, 5, 6, 7, 8, 9, or 10 CSBM per week after 1 or more, 2 or more, 3 or more, 4 or more, 5 or more, 6 or more, or 7 or more weeks of treatment. In a further aspect, a treated subject's number of CSBM per week is maintained for at least 1, 2, 3, 4, 5, 6, 7, 8, 9, 10, 11, 12, 13, 14, 15, 16, 17, 18, 19, 20, 21, 22, 23, or 24 weeks after the completion of treatment. In another aspect, a treated subject's number of CSBM per week increases by at least 1, 2, 3, 4, 5, 6, 7, 8, 9, or 10 CSBM per week at 4, 8, 16, and 32 weeks compared to baseline.

In one aspect, a symptom severity reduction (e.g., for ASD symptoms, GI symptoms, or both) is ongoing during a treatment or sustained after finishing or discontinuing a treatment. In one aspect, a symptom severity reduction (e.g., for ASD symptoms, GI symptoms, or both) is assessed at a specific time point during or post treatment, e.g., about 2, 4, 6, 8, 12, 18, 24, 32, 40, 48 weeks after initiating a treatment, or about 2, 4, 6, 8, 12, 18, 24, 32, 40, 48 weeks after finishing or discontinuing a treatment.

In one aspect, a method further comprises administering an antibiotic to a subject prior to administering a pharmaceutical composition comprising a fecal microbe preparation. In another aspect, a method further comprises subjecting a subject to a bowel cleanse.

In another aspect, provided herein is a method of treating an autism spectrum disorder in a human subject. In exemplary aspects, the method comprises or consists essentially of the following steps: administering an antibiotic to a human subject; subjecting the human subject to a bowel cleanse after administering the antibiotic; and administering a pharmaceutical composition described herein to the human subject after the bowel cleanse, wherein an autism spectrum disorder is treated in the human subject.

In exemplary aspects, treating ASD comprises alleviating, ameliorating, delaying the onset of, inhibiting the progression of, or reducing the severity of one or more, two or more, three or more, four or more, five or more, six or more, seven or more, eight or more symptoms characteristic of ASD. In one aspect, a treatment alleviates, ameliorates, delays the onset of, inhibits the progression of, or reduces the severity of one or more social and cognitive core ASD-related symptoms. In some aspects, the symptom(s) is selected from the group consisting of: (i) insistence on sameness or resistance to change; (ii) difficulty in expressing needs; (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play; (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues. In some aspects, the symptom(s) is selected from the group consisting of compulsive behavior, ritualistic behavior, restricted behavior, stereotypy, sameness, or self-injury. The methods described here can lead to improvement of any combination of the foregoing symptoms.

In exemplary aspects, the human subject exhibits a significant reduction in autism symptom severity as assessed according to a ASD rating scale. In some cases, for example, the human subject exhibits at least a 10% or 20% reduction in autism symptom severity as assessed by the Childhood Autism Rating Scale (CARS) relative to severity as assessed prior to initiating the method.

Subjects appropriate for treatment according to a method provided herein may not present with or report gastrointestinal distress symptoms prior to initiating a method as provided herein. In some cases, for example, a human subject appropriate for treatment according to a method provided herein manifests no gastrointestinal symptoms prior to or at the time at which treatment is begun. In one aspect, an ASD subject treated herein exhibit one or more or two or more GI symptoms selected from the group consisting of abdominal pain, reflux, indigestion, irritable bowel syndrome, chronic persistent diarrhoea, diarrhoea, flatulence, constipation, and alternating constipation/diarrhoea.

Regardless of the presence or absence of gastrointestinal distress symptoms, human subjects appropriate for the methods provided herein typically have significantly fewer species of gut bacteria before said method of treatment as compared to a neurotypical human. In some cases, the human subject to be treated by the method exhibits at least about 20%, 30%, 40%, 50%, 60%, 70%, 80%, or 90% fewer species of gut bacteria prior to administration of the pharmaceutical composition as compared to a neurotypical human.

In one aspect, a treated subject has reduced adverse events during treatment. In another aspect, a treated subject has no adverse events during treatment. In an aspect of the present disclosure, an adverse event is selected from the group consisting of abdominal cramping, fullness, flatulence, bloating, diarrhea, blood in stool, fever, and a combination thereof. In another aspect, an adverse event is any signs or symptoms, regardless of severity, any clinically significant laboratory abnormality, or any abnormality detected during physical examination. In yet another aspect, an adverse event is ascribed to the pharmaceutically active dose. In a further aspect, an adverse event is not ascribed to the pharmaceutically active dose. In yet another aspect, an adverse event comprises a solicited adverse event, an unsolicited adverse event, a serious adverse event, or a combination thereof. In an aspect, serious adverse events require inpatient hospitalization or prolongation of existing hospitalization; result in persistent or significant disability and/or incapacity, result in a congenital anomaly and/or birth defect; or is any important medical event, based on medical and scientific judgment, which may not be immediately life-threatening or result in death or hospitalization, but may pose substantial risks to the patient or may require medical intervention to prevent 1 of the other outcomes listed above.

In an aspect of the present disclosure, a treated subject has reduced or no adverse events through 2, 4, 6, 8, 10, 12, 14, 16, 18, 20, 22, 24, 26, 28, 30, or 32 weeks of treatment.

Also provided herein are methods for reducing autism severity in an autistic human subject. In exemplary aspects, the method comprises or consists essentially of the following steps: orally-administering a non-absorbable antibiotic to an autistic human subject; subjecting the autistic human subject to a bowel cleanse; and administering a bacterial mixture comprising one or more bacterial isolates and a preparation of uncultured fecal bacteria from a neurotypical human donor to the human subject, wherein the human subject exhibits a significant reduction in autism symptom severity as assessed by the Childhood Autism Rating Scale (CARS) after said method as compared to before initiating the method. In some cases, the human subject exhibits at least a 10% or 20% reduction in autism symptom severity as assessed by the Childhood Autism Rating Scale (CARS) relative to severity as assessed prior to initiating the method.

In one aspect, the present disclosure provides a method for treating ASD in a subject in need thereof, where the method comprises orally administering to the subject a pharmaceutically active dose of a pharmaceutical composition described herein, wherein the pharmaceutically active dose is administered with at least 50 ml of water. In another aspect, a method comprises administering a bacterial mixture no less than 2 hours after consumption of food or liquids besides water. In yet another aspect, a method comprises consumption of food or water no less than one hour after administering a bacterial mixture. In an aspect, the present disclosure provides a method for treating ASD in a subject in need thereof by administering a pharmaceutical composition described herein, wherein the method comprises administering the pharmaceutical composition at least 2 hours after any solid or liquid caloric intake. In another aspect, the method comprises administering the pharmaceutical composition at least 1 hour prior to any solid or liquid caloric intake.

In one aspect, the present disclosure provides a method for treating ASD in a subject in need thereof comprising administering to the subject an amount of a pharmaceutical composition effective at providing at least a 10% improvement in assessment score. In an aspect, the pharmaceutical composition comprises a bacterial mixture comprising a preparation of uncultured fecal bacteria (e.g., a substantially complete fecal microbiota) and one or more bacterial isolates. In another aspect, the subject has a GI symptom of constipation with less than 3 complete spontaneous bowel movements per week for a period of time. In yet another aspect, the subject exhibits an improvement in assessment score after the treatment as compared to before initiating the treatment, and wherein the assessment score is based on an assessment system selected from the group consisting of Clinical Global Impressions Scale (CGI), Children's Yale Brown Obsessive Compulsive Scale (CY-BOCS), Aberrant Behavior Checklist (ABC), Vineland Adaptive Behavior Scale II (VABS-II) and VABS-III.

In one aspect, the present disclosure provides a method for treating ASD in a subject in need thereof by administering an amount of a pharmaceutical composition described herein, where the subject in need thereof has a GI symptom of constipation with less than 3 complete spontaneous bowel movements per week. In another aspect, the subject in need thereof has a GI symptom of constipation with less than 2 complete spontaneous bowel movements per week. In a further aspect, the subject in need thereof has a GI symptom of constipation with less than 2 complete spontaneous bowel movements per week. In yet another aspect, the subject in need thereof has a GI symptom of constipation with less than 1 complete spontaneous bowel movement per week. In an aspect, the subject in need thereof has a GI symptom of constipation for a period of time selected from the group consisting of about 1, 2, 3, and 4 weeks. In another aspect, the subject in need thereof has a GI symptom of constipation for a period of time selected from the group consisting of about 10, 20, 30, and 40 days. In another aspect, the subject in need thereof has a GI symptom of constipation for a period of between 10 and 15, 15 and 20, 20 and 25, 25 and 30, 30 and 35, 35 and 40 days.

In one aspect, the present disclosure provides a method for treating ASD in a subject in need thereof by administering a pharmaceutical composition described herein, where the subject in need thereof has a GI symptom of constipation which improves by at least 1, 2, 3, 4, 5, 6, 7, 8, 9, or 10 complete spontaneous bowel movements (CSBM) per week after at least 1, 2, 3, 4, 5, 6, 7, or 8 weeks of treatment. In another aspect, the subject in need thereof has a GI symptom of constipation which improves by at least 1, 2, 3, 4, 5, 6, 7, 8, 9, or 10 complete spontaneous bowel movements (CSBM) per week after between 1 or more, 2 or more, 3 or more, 4 or more, 5 or more, 6 or more, or 7 or more weeks of treatment. In a further aspect, the subject in need thereof has a GI symptom of constipation which remains improved for at least 1, 2, 3, 4, 5, 6, 7, or 8 weeks after the completion of treatment.

In one aspect, the present disclosure provides a method for treating ASD in a subject in need thereof by administering a pharmaceutical composition described herein, where the subject in need thereof has a GI symptom of constipation with less than 3 complete spontaneous bowel movements per week and exhibits an ASD qualifying assessment score before treatment. In an aspect, the qualifying assessment score is based on an assessment system selected from the group consisting of Clinical Global Impressions Scale (CGI), Children's Yale Brown Obsessive Compulsive Scale (CY-BOCS), Aberrant Behavior Checklist (ABC), Autism Diagnostic Interview-Revised (ADI-R), Vineland Adaptive Behavior Scale II (VABS-II) and VABS-III.

In an aspect, the present disclosure provides a method for treating ASD in a subject in need thereof by administering a pharmaceutical composition described herein, where the subject in need thereof has a CGI-S score of 2, 3, 4, 5, 6, or 7 before treatment. In another aspect, the subject in need thereof has a CGI-S score of 2 or higher, 3 or higher, 4 or higher, 5 or higher, or 6 or higher before treatment. In yet another aspect, the subject in need thereof further has a GI symptom comprising constipation with less than 3 CSBM per week.

In an aspect, the present disclosure provides a method for treating ASD in a subject in need thereof by administering a pharmaceutical composition described herein, wherein the subject has a CGI-I score of 1, 2, 3, or 4 after one or more weeks of treatment. In another aspect, the patient in need thereof has a CGI-I score of 1, 2, 3, or 4 after two or more weeks of treatment. In a further aspect, the subject in need thereof has a CGI-I score of 1, 2, 3, or 4 after three or more weeks of treatment. In another aspect, the subject in need thereof has a CGI-I score of 1, 2, 3, or 4 after four or more weeks of treatment. In another aspect, the subject in need thereof has a CGI-I score of 1, 2, 3, or 4 after five or more weeks of treatment. In a further aspect, the subject in need thereof has a CGI-I score of 1, 2, 3, or 4 after 6 or more weeks of treatment. In another aspect, the subject in need thereof has a CGI-I score of 1, 2, 3, or 4 after 7 or more weeks of treatment. In another aspect, the subject in need thereof has a CGI-I score of 1, 2, or 3 after 2 or more weeks of treatment. In yet another aspect, the subject in need thereof has a CGI-I score that improves from 5 to 4, 5 to 3, 5 to 2, 5 to 1, 4 to 3, 4 to 2, 4 to 1, 3 to 2, 3 to 1, or 2 to 1 after at least 1, 2, 3, 4, 5, 6, 7, or 8 weeks of treatment. In another aspect, the subject in need thereof has a CGI-I score that improves from 5 to 4, 5 to 3, 5 to 2, 5 to 1, 4 to 3, 4 to 2, 4 to 1, 3 to 2, 3 to 1, or 2 to 1 after 1 or more, 2 or more, 3 or more, 4 or more, 5 or more, 6 or more, 7 or more, or 8 weeks of treatment. In another aspect, the subject in need thereof has a CGI-I score that improves by at least 1, 2, 3, or 4 points after 1 or more, 2 or more, 3 or more, 4 or more, 5 or more, 6 or more, 7 or more, or 8 weeks of treatment.

In an aspect, the present disclosure provides a method for treating ASD in a subject in need thereof by administering a pharmaceutical composition described herein, wherein the treatment is effective at providing at least a 10% improvement in one or more assessment scores selected from the group consisting of CARS-2, ABC-2, RIAS-NV, SRS-2, and PGI-III. In an aspect, the subject in need thereof has a GI symptom comprising constipation with less than 3 CSBM per week prior to the start of the treatment. In an aspect, the 10% improvement in one or more assessments scores is after at least 1, 2, 3, 4, 5, 6, 7, 8, 9, 10, 11, 12, 13, 14, 15, or 16 weeks of treatment. In another aspect, the treatment is effective at providing at least a 10% improvement in two or more assessment scores selected from the group consisting of CARS-2, ABC-2, RIAS-NV, SRS-2, and PGI-III. In another aspect, the treatment is effective at providing at least a 10% improvement in three or more assessment scores selected from the group consisting of CARS-2, ABC-2, RIAS-NV, SRS-2, and PGI-III. In another aspect, the treatment is effective at providing at least a 10% improvement in four or more assessment scores selected from the group consisting of CARS-2, ABC-2, RIAS-NV, SRS-2, and PGI-III.

In an aspect, the present disclosure provides a method for treating ASD in a subject in need thereof by administering a pharmaceutical composition described herein, wherein the method comprises analyzing the subject's metabolite profile in blood, stool, or urine before, during, and after treatment. In another aspect, the method further comprises analyzing a subject's metabolite profile in blood, stool or urine at least twice during treatment and at least once post-treatment. In another aspect, the method further comprises analyzing the subject's metabolite profile in blood prior to initiating the treatment.

In an aspect of the present disclosure, the subject in need thereof is between the age of 5 and 17. In another aspect, the subject in need thereof is at least 5 years old. In another aspect, the subject in need thereof is younger than 17 years old.

In another aspect, the subject in need thereof does not have any serious medical disorders requiring medication dose adjustments, wherein the serious medical disorder is selected from the group consisting of single-gene disorder, major brain malformation, tube feeding, severe GI problems that require immediate treatment (life-threatening), diagnosed Celiac Disease, Eosinophilic Gastroenteritis, severely underweight/malnourished, and recent/scheduled surgeries.

In an aspect, the present disclosure provides a method for screening an individual for adherence by administering capsules containing placebo for at least 7 consecutive days. In another aspect, placebo capsules are administered for at least 14 consecutive days.

In an aspect, the present disclosure provides a method for treating an autism spectrum disorder (ASD) in a subject in need thereof, where the method comprises administering to the subject an amount of a pharmaceutical composition described herein, effective at providing at least a 10% improvement in Children's Yale Brown Obsessive Compulsive Scale (CY-BOCS) assessment score after treatment as compared to before initiating the treatment. In an aspect, the pharmaceutical composition is effective at providing at least a 10% improvement in CY-BOCS assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 10% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 10% improvement in CY-BOCS assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 10% improvement in CY-BOCS assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 10% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 15% improvement in CY-BOCS assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 15% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 15% improvement in CY-BOCS assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 15% improvement in CY-BOCS assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 15% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 20% improvement in CY-BOCS assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 20% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 20% improvement in CY-BOCS assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 20% improvement in CY-BOCS assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 20% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 30% improvement in CY-BOCS assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 30% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 30% improvement in CY-BOCS assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 30% improvement in CY-BOCS assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 30% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 40% improvement in CY-BOCS assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 40% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 40% improvement in CY-BOCS assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 40% improvement in CY-BOCS assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 40% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In another aspect, the subject in need thereof has a CY-BOCS assessment score of 8 or higher before the treatment. In another aspect, the subject in need thereof has a CY-BOCS assessment score of 16 or higher before the treatment. In an aspect, the subject in need thereof further comprises a GI symptom comprising constipation with less than 3 CSBM per week prior to the start of the treatment.

In an aspect, the subject in need thereof has a CY-BOCS score of 8 to 15, 16 to 23, 24 to 31, or 32 to 40 before treatment. In an aspect, the subject in need thereof has a CY-BOCS score of 0 to 7, 8 to 15, or 16 to 23, after at least 1, 2, 3, 4, 5, 6, 7, or 8 weeks of treatment. In another aspect, the subject in need thereof has a CY-BOCS score of 0 to 7, 8 to 15, 16 to 23, after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the subject in need thereof has a CY-BOCS score which improves from severe or extreme to moderate, mild, or subclinical after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In yet another aspect, the subject in need thereof has a CY-BOCS score that improves by at least 1 severity range after 1 or more, 2 or more, 3 or more, 4 or more, 5 or more, 6 or more, 7 or more, or 8 weeks of treatment. In another aspect, the subject in need thereof has a CY-BOCS score that improves by at least 2 severity ranges after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the subject in need thereof has a CY-BOCS score that improves by at least 3 severity ranges after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In yet another aspect, the subject in need thereof has a CY-BOCS score that improves by at least 4 severity ranges after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In a further aspect, the subject in need thereof has a CY-BOCS score that is maintained for at least 1, 2, 3, 4, 5, 6, 7, 8, 9, 10, 11, 12, 13, 14, 15, or 16 weeks of treatment.

In an aspect, the pharmaceutical composition is effective at providing at least a 10% improvement in ABC assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 10% improvement in ABC assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 10% improvement in ABC assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 10% improvement in ABC assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 10% improvement in ABC assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 15% improvement in ABC assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 15% improvement in ABC assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 15% improvement in ABC assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 15% improvement in ABC assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 15% improvement in ABC assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 20% improvement in ABC assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 20% improvement in ABC assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 20% improvement in ABC assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 20% improvement in ABC assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 20% improvement in ABC assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 30% improvement in ABC assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 30% improvement in ABC assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 30% improvement in ABC assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 30% improvement in ABC assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 30% improvement in ABC assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 40% improvement in ABC assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 40% improvement in ABC assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 40% improvement in ABC assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 40% improvement in ABC assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 40% improvement in ABC assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 10% improvement in CARS-2 assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 10% improvement in CARS-2 assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 10% improvement in CARS-2 assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 10% improvement in CARS assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 10% improvement in CARS-2 assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 15% improvement in CARS-2 assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 15% improvement in CARS-2 assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 15% improvement in CARS-2 assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 15% improvement in CARS-2 assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 15% improvement in CARS-2 assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 20% improvement in CARS-2 assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 20% improvement in CARS-2 assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 20% improvement in CARS-2 assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 20% improvement in CARS-2 assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 20% improvement in CARS-2 assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 30% improvement in CARS-2 assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 30% improvement in CARS-2 assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 30% improvement in CARS-2 assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 30% improvement in CARS-2 assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 30% improvement in CARS-2 assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 40% improvement in CARS-2 assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 40% improvement in CARS-2 assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 40% improvement in CARS-2 assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 40% improvement in CARS-2 assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 40% improvement in CARS-2 assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the present disclosure provides a method for treating ASD in a subject in need thereof by administering a pharmaceutical composition described herein, wherein the pharmaceutical composition is effective at providing at least a 10% improvement in a Vineland Adaptive Behavior Scale II (VABS-II) assessment score after the treatment as compared to before initiating the treatment. In an aspect, the pharmaceutical composition is effective at providing at least a 10% improvement in VABS-II or VABS-III assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 10% improvement in VABS-II or VABS-III assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 10% improvement in VABS-II or VABS-III assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 10% improvement in VABS-II or VABS-III assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 15% improvement in VABS-II or VABS-III assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 15% improvement in VABS-II or VABS-III assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 15% improvement in VABS-II or VABS-III assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 15% improvement in VABS-II or VABS-III assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 15% improvement in VABS-II or VABS-III assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 20% improvement in VABS-II or VABS-III assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 20% improvement in VABS-II or VABS-III assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 20% improvement in VABS-II or VABS-III assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 20% improvement in VABS-II or VABS-III assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 20% improvement in VABS-II or VABS-III assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 30% improvement in VABS-II or VABS-III assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 30% improvement in VABS-II or VABS-III assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 30% improvement in VABS-II or VABS-III assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 30% improvement in VABS-II or VABS-III assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 30% improvement in VABS-II or VABS-III assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 40% improvement in VABS-II or VABS-III assessment score after at least 1, 2, 3, 4, 5, 6, 7, 8, 9, 10, 11, 12, 13, 14, 15, or 16 weeks of treatment. In another aspect, the pharmaceutical composition is effective at providing at least a 40% improvement in VABS-II or VABS-III assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 40% improvement in VABS-II or VABS-III assessment score between 1 and 3, 3 and 5, 5 and 7, 7 and 9, 9 and 11, 11 and 13, 13 and 16 weeks of treatment. In another aspect, the pharmaceutical composition is effective at maintaining at least a 40% improvement in VABS-II or VABS-III assessment score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 40% improvement in VABS-II or VABS-III assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the subject in need thereof exhibits a VABS-II domain assessment score of 85 or lower and at least one subdomain score of 12 or lower prior to initiating the treatment. In another aspect, the subject in need thereof exhibits a VABS-II domain assessment score of 75 or lower and at least one subdomain assessment score of 12 or lower prior to initiating the treatment. In another aspect, the subject in need thereof exhibits a VABS-II domain assessment score of 65 or lower and at least one subdomain assessment score of 12 or lower prior to initiating the treatment. In another aspect, the pharmaceutical composition is effective at providing at least a 10% improvement in a VABS-II assessment score after 1 or more, 2 or more, 3 or more, 4 or more, 5 or more, 6 or more, 7 or more, or 8 weeks of treatment compared to before initiating the treatment. In yet another aspect, the 10% improvement in a VABS-II assessment score is maintained for at least 1, 2, 3, 4, 5, 6, 7, 8, 9, 10, 11, 12, 13, 14, 15, 16, 17, 18, 19, 20, 21, 22, 23, or 24 weeks after treatment has completed.

In an aspect, the present disclosure provides a method for treating an autism spectrum disorder (ASD) in a subject in need thereof, where the method comprises administering to the subject an amount of a pharmaceutical composition described herein, effective at providing at least a 10% improvement in Reynolds Intellectual Assessment Scales™—Nonverbal (RIAS-NV) IQ score after treatment as compared to before initiating the treatment. In an aspect, the pharmaceutical composition is effective at providing at least a 10% improvement in RIAS-NV score assessment score after at least 1, 2, 3, 4, 5, 6, 7, 8, 9, 10, 11, or 12 weeks of treatment. In another aspect, the pharmaceutical composition is effective at providing at least a 10% improvement in RIAS-NV score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 10% improvement in RIAS-NV score between 1 and 3, 3 and 5, 5 and 7, 7 and 9, 9 and 11, 11 and 13, 13 and 16 weeks of treatment. In another aspect, the pharmaceutical composition is effective at maintaining at least a 10% improvement in RIAS-NV score at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 10% improvement in RIAS-NV score 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 15% improvement in RIAS-NV score after at least 1, 2, 3, 4, 5, 6, 7, 8, 9, 10, 11, 12, 13, 14, 15, or 16 weeks of treatment. In another aspect, the pharmaceutical composition is effective at providing at least a 15% improvement in RIAS-NV score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 15% improvement in RIAS-NV score between 1 and 3, 3 and 5, 5 and 7, 7 and 9, 9 and 11, 11 and 13, 13 and 16 weeks of treatment. In another aspect, the pharmaceutical composition is effective at maintaining at least a 15% improvement in RIAS-NV score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 15% improvement in CY-BOCS assessment score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 20% improvement in RIAS-NV score after at least 1, 2, 3, 4, 5, 6, 7, 8, 9, 10, 11, 12, 13, 14, 15, or 16 weeks of treatment. In another aspect, the pharmaceutical composition is effective at providing at least a 20% improvement in RIAS-NV score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 20% improvement in RIAS-NV score between 1 and 3, 3 and 5, 5 and 7, 7 and 9, 9 and 11, 11 and 13, 13 and 16 weeks of treatment. In another aspect, the pharmaceutical composition is effective at maintaining at least a 20% improvement in RIAS-NV score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 20% improvement in RIAS-NV score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 30% improvement in RIAS-NV score after at least 1, 2, 3, 4, 5, 6, 7, 8, 9, 10, 11, 12, 13, 14, 15, or 16 weeks of treatment. In another aspect, the pharmaceutical composition is effective at providing at least a 30% improvement in RIAS-NV score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 30% improvement in RIAS-NV score between 1 and 3, 3 and 5, 5 and 7, 7 and 9, 9 and 11, 11 and 13, 13 and 16 weeks of treatment. In another aspect, the pharmaceutical composition is effective at maintaining at least a 30% improvement in RIAS-NV score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 30% improvement in RIAS-NV score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the pharmaceutical composition is effective at providing at least a 40% improvement in RIAS-NV score after at least 1, 2, 3, 4, 5, 6, 7, 8, 9, 10, 11, 12, 13, 14, 15, or 16 weeks of treatment. In another aspect, the pharmaceutical composition is effective at providing at least a 40% improvement in RIAS-NV score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 weeks of treatment. In another aspect, the pharmaceutical composition is effective at providing at least a 40% improvement in RIAS-NV score between 1 and 3, 3 and 5, 5 and 7, 7 and 9, 9 and 11, 11 and 13, 13 and 16 weeks of treatment. In another aspect, the pharmaceutical composition is effective at maintaining at least a 40% improvement in RIAS-NV score after at least 1, 2, 3, 4, 5, 6, 7, 8, 9, 10, 11, 12, 13, 14, 15, 16, 17, 18, 19, 20, 21, 22, 23, or 24 weeks after treatment has completed. In yet another aspect, the pharmaceutical composition is effective at maintaining at least a 40% improvement in RIAS-NV score after 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, or 16 or more, 17 or more, 18 or more, 19 or more, 20 or more, 21 or more, 22 or more, 23 or more, or 24 or more weeks after treatment has completed.

In an aspect, the present disclosure provides a method for treating ASD in a subject in need thereof by administering a pharmaceutical composition described herein, wherein the pharmaceutical composition is effective at providing at least a 10% improvement in assessment score, wherein the subject exhibits the criteria for autistic spectrum disorder identified by the Autism Diagnostic Interview-Revised (ADI-R) assessment. In another aspect, the subject in need thereof exhibits the criteria for autistic spectrum disorder identified by the ADI-R assessment and further comprises a GI symptom of constipation with less than 3 CSBM per week. In another aspect, the subject in need thereof exhibits the criteria for autistic spectrum disorder identified by the ADI-R assessment and the at least 10% improvement in assessment score is based on an assessment system selected from the group consisting of CARS-2, ABC-2, RIAS-NV, SRS-2, and PGI-III In another aspect, the subject in need thereof exhibits the criteria for autistic spectrum disorder identified by the ADI-R assessment and the at least 10% improvement in assessment score is based on an assessment system selected from the group consisting of CGI, CY-BOCS, ABC, VABS-II, and VABS-III.

In one aspect, the present disclosure provides a method for treating ASD in a subject in need thereof, where the method comprises administering to the subject a pharmaceutically active or therapeutically effective dose of a pharmaceutical composition described herein. In one aspect, the present disclosure provides a method for treating ASD in a subject in need thereof, where the method comprises administering daily to the subject a therapeutically effective dose of a pharmaceutical composition described herein. In one aspect, a pharmaceutical composition is administered to a patient in need thereof at least once daily for at least two consecutive days. In one aspect, a pharmaceutical composition is administered at least once daily for at least 3, 4, 5, 6, 7, 8, 9, 10, 11, 12, 13, 14, or 15 consecutive days. In another aspect, a pharmaceutical composition is administered at least once daily for at least 1, 2, 3, 4, 5, 6, 7, 8, 9, 10, 11, 12, 13, 14, 15 or 16 consecutive weeks. In one aspect, a pharmaceutical composition is administered at least once daily for at most 4, 5, 6, 7, 8, 9, 10, 11, 12, 13, 14, 15, 16, 17, 18, 19, or 20 consecutive days or weeks. In another aspect, a pharmaceutical composition is administered at least once daily for at most 1, 2, 3, 4, 5, 6, 7, 8, 9, 10, 11, 12, 13, 14, 15 or 16 consecutive weeks or months. In a further aspect, a pharmaceutical composition is administered at least once for at least 1, 2, 3, 4, 5, 6, 7, 8, 9, 10, 11, 12, 13, 14, 15 or 16 consecutive months or years, chronically for a subject's entire life span, or an indefinite period of time.

In one aspect, a pharmaceutical composition is administered to a patient in need thereof at least twice daily for at least two consecutive days. In one aspect, a pharmaceutical composition is administered at least twice daily for at least 3, 4, 5, 6, 7, 8, 9, 10, 11, 12, 13, 14, or 15 consecutive days. In another aspect, a pharmaceutical composition is administered at least twice daily for at least 1, 2, 3, 4, 5, 6, 7, 8, 9, 10, 11, 12, 13, 14, 15 or 16 consecutive weeks. In one aspect, a pharmaceutical composition is administered at least twice daily for at most 4, 5, 6, 7, 8, 9, 10, 11, 12, 13, 14, 15, 16, 17, 18, 19, or 20 consecutive days or week. In another aspect, a pharmaceutical composition is administered at least twice daily for at most 1, 2, 3, 4, 5, 6, 7, 8, 9, 10, 11, 12, 13, 14, 15 or 16 consecutive weeks or months. In a further aspect, a pharmaceutical composition is administered at least twice for at least 1, 2, 3, 4, 5, 6, 7, 8, 9, 10, 11, or 12 consecutive months or years, chronically for a subject's entire life span, or an indefinite period of time.

In one aspect, a pharmaceutical composition is administered to a patient in need thereof at least three times daily for at least two consecutive days. In one aspect, a pharmaceutical composition is administered at least three times daily for at least 3, 4, 5, 6, 7, 8, 9, 10, 11, 12, 13, 14, or 15 consecutive days. In another aspect, a pharmaceutical composition is administered at least three times daily for at least 1, 2, 3, 4, 5, 6, 7, 8, 9, 10, 11, 12, 13, 14, 15 or 16 consecutive weeks. In one aspect, a pharmaceutical composition is administered at least three times daily for at most 4, 5, 6, 7, 8, 9, 10, 11, 12, 13, 14, 15, 16, 17, 18, 19, or 20 consecutive days or weeks. In another aspect, a pharmaceutical composition is administered at least three times daily for at most 1, 2, 3, 4, 5, 6, 7, 8, 9, 10, 11, 12, 13, 14, 15 or 16 consecutive weeks or months. In a further aspect, a pharmaceutical composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating ASD in a subject in need thereof, where the method comprises administering orally to the subject a therapeutically active dose of a pharmaceutical composition comprising a bacterial mixture, where the dose is administered at a dosing schedule of at least once or twice daily for at least three consecutive days or weeks. In another aspect, a dose is administered at least once, twice, or three times daily for a period between 1 and 16 weeks, between 2 and 16 weeks, between 3 and 16 weeks, between 4 and 16 weeks, between 5 and 16 weeks, between 6 and 16 weeks, between 7 and 16 weeks, between 8 and 16 weeks, between 10 and 16 weeks, between 12 and 16 weeks, between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating ASD in a subject in need thereof by administering a pharmaceutical composition described herein, where the method comprises a single dosing schedule. In one aspect, the dosing schedule comprises a treatment period of at least 1, 2, 3, 4, 5, 6, 7, 8, 9, 10, 11, 12, 13, 14, 15, or 16 consecutive weeks. In an aspect, a dosing schedule comprises administering a dose every day, every other day, every two days, or every 3, 4, 5, 6, 7, 8 days.

In one aspect, the present disclosure provides a method for treating ASD in a subject in need thereof by administering a pharmaceutical composition described herein, where the method comprises a first dosing schedule followed by a second dosing schedule. In one aspect, a first dosing schedule comprises a treatment or induction dose. In one aspect, a first dosing schedule comprises a continuous dosing schedule. In another aspect, a first dosing schedule comprises a dosing schedule of two consecutive days. In another aspect, a first dosing schedule comprises a dosing schedule of two consecutive days of an equivalent dose. In another aspect, a first dosing schedule comprises a dose on a single day. In another aspect, a first dosing schedule comprises a dosing schedule of three consecutive days. In another aspect, a first dosing schedule comprises a dosing schedule of four consecutive days. In another aspect, a first dosing schedule comprises a dosing schedule of five consecutive days. In another aspect, a first dosing schedule comprises a dosing schedule of six consecutive days. In another aspect, a first dosing schedule comprises a dosing schedule of seven consecutive days. In another aspect, a first dosing schedule comprises a dosing schedule of at least 3, 4, 5, 6, 7, 8, 9, 10, 11, or 12 consecutive days. In another aspect, a second dosing schedule comprises a maintenance dose lower than or equal to a pharmaceutically active dose of a first dosing schedule. In another aspect, a second dosing schedule lasts for at least about 2, 4, 5, 6, 7, 8, 9, 10, 11, 12, 18, 24, 36, 48, 72, or 96 weeks. In another aspect, a second dosing schedule comprises a dosing schedule of at least 2, 4, 5, 6, 7, 8, 9, 10, 11, 12, 18, 24, 36, 48, 72, or 96 consecutive weeks. In another aspect, a second dosing schedule comprises a dosing schedule of at least 2, 4, 5, 6, 7, 8, 9, 10, 11, 12, 18, 24, 36, 48, 72, or 96 consecutive weeks. In another aspect, a second dosing schedule comprises a dosing schedule of at least 12, 14, 21, 28, 35, 42, 49, 56, 63, 70, or 77 consecutive days. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, there is no interval between a first and a second dosing schedule. In another aspect, the interval between a first and a second dosing schedule is at least 1, 2, 3, 4, 5, 6, or 7 days. In one aspect, the interval between a first and a second dosing schedule is at least about 1, 2, 3, 4, 5, 6, 7, 8, 9, 10, 11, 12, 13, 14, 15 or 16 weeks. In another aspect, a second dosing schedule (e.g., a maintenance dose) comprises a dosage about 2, 3, 4, 5, 10, 50, 100, 200, 400, or 500 folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, the present disclosure provides a method for treating a subject in need thereof, where the method comprises administering to the subject a pharmaceutically active dose of a pharmaceutical composition comprising a bacterial mixture that comprises a preparation of uncultured fecal bacteria of multiple carefully screened, healthy donors. In an aspect, a subject is administered a pharmaceutical composition over a dosing period wherein a first dose comprises at least one pharmaceutical composition comprising a preparation of uncultured fecal bacteria of a single donor, and a second dose of a pharmaceutical composition comprising a preparation of uncultured fecal bacteria of a single donor different from the donor of the first dose. In another aspect, a first dose comprises a pharmaceutical composition comprising a preparation of uncultured fecal bacteria of a single donor and a second dose comprises a preparation of uncultured fecal bacteria of a donor pool. The first and second dose do not indicate the order of administration to a subject, but rather that preparation of uncultured fecal bacteria from separate donors may be used in a non-blended form. In yet another aspect, the preparation of uncultured fecal bacteria from multiple carefully screened, healthy donors is provided in a blended form.

In an aspect, a pharmaceutical composition used herein comprises a bacterial mixture comprising a preparation of uncultured fecal bacteria derived from a donor with preselected desirable characteristics or receiving certain pre-treatment(s). In an aspect, a donor has no current or previous ASD diagnosis or has no ASD symptom. In another aspect, a donor has no family member or direct relative diagnosed with ASD or exhibiting an ASD symptom. In another aspect, a donor has no siblings, parents, or children diagnosed with ASD or exhibiting an ASD symptom. In an aspect, a donor has not previously received any fecal microbiota transplantation. In an aspect, a fecal donor for ASD treatment previously donated a stool for treating a GI disorder, e.g., a C. difficile infection or Inflammatory Bowel Disease (IBD).

In an aspect, a subject receiving a treatment described here is a pregnant woman. In another aspect, a pregnant woman receiving a treatment here has an older child diagnosed with ASD or exhibiting an ASD syndrome. In another aspect, a pregnant woman receiving a treatment here is at risk for giving birth to a child with ASD. In another aspect, a fecal donor is pregnant. In a further aspect, a preparation of uncultured fecal bacteria prepared from a stool from a pregnant donor is administered to a pregnant subject (e.g., in combination with one or more bacterial isolates) at risk for giving birth to a child with ASD.

In an aspect, a method of treating a subject with ASD comprises administering to the subject a pharmaceutical composition comprising a bacterial mixture comprising a member of the genus Lactobacillus. For example, a bacterial mixture administered to a subject in a pharmaceutical composition can comprise L. reuteri (e.g., in a preparation of uncultured fecal bacteria and/or a bacterial isolate included within the bacterial mixture). Administration of certain strains of bacteria (e.g. L. reuteri) to a subject exhibiting one or more symptoms of ASD, or susceptible to developing one or more symptoms of ASD, can treat or prevent the ASD. Without being bound by theory, following administration of L. reuteri to a subject, the L. reuteri can engraft in the intestine of the subject and act to treat or prevent ASD via an oxytocin-dependent mechanism of action. For example, L. reuteri can stimulate neuronal signaling through afferent vagus nerve connections from the intestinal mucosa to a brain region (e.g. paraventricular nucleus, or PVN) that affects behavior of the subject. In an aspect, L. reuteri can induce oxytocin release by PVN neurons and modulate synaptic plasticity in dopaminergic neurons through the oxytocin receptor which modulates social behavior.

In an aspect, disclosed herein is a method of engrafting a member of the genus Lactobacillus (e.g., L. reuteri) in an intestine of a subject, the method comprising administering to the subject a pharmaceutical composition comprising a bacterial mixture comprising (i) a preparation of uncultured fecal bacteria; and (ii) a bacterial isolate comprising the member of Lactobacillus; wherein engraftment of the member of Lactobacillus in an intestine of the subject after administering the composition is greater than engraftment prior to administering the composition. Engraftment can be measured, for example, by determining a relative abundance of the member of Lactobacillus in an intestinal microbiota of the subject, for example a fecal microbiota. In an aspect, a relative abundance of the member of Lactobacillus in the intestinal microbiota is greater following administration of both the preparation of uncultured fecal bacterial and the bacterial isolate comprising the member of Lactobacillus than a relative abundance of Lactobacillus in the intestinal microbiota when the bacterial isolate is administered alone. That is, administering the preparation of uncultured fecal bacteria in combination with the bacterial isolate comprising the member of Lactobacillus facilitates, promotes and/or synergizes the engraftment of the L. reuteri bacterial isolate in the intestine of the subject.

In an aspect, the present disclosure provides for methods for treating a subject in need thereof by administering to the subject a pharmaceutically active dose of a pharmaceutical composition comprising a preparation of uncultured fecal bacteria of a single donor, and optionally one or more bacterial isolates. In another aspect, the administering is followed by testing to determine the efficacy of the pharmaceutically active dose of the pharmaceutical composition. In another aspect, the testing of the subject provides results to determine if the active dose of the pharmaceutical composition should be adjusted. In another aspect, the testing is followed by administration of a pharmaceutical composition comprising a preparation of uncultured fecal bacteria blended from multiple donors, and optionally one or more microbial isolates. In one aspect of the present disclosure, methods provide for treating a subject in need thereof comprising: (1) administering to the subject a first pharmaceutically active dose of a pharmaceutical composition comprising a preparation of uncultured fecal bacteria of a single donor (and optionally one or more bacterial isolates); (2) testing of the subject to determine efficacy, if an additional dose is necessary, or if the dose should be adjusted; (3) administration of a second pharmaceutical composition comprising a preparation of uncultured fecal bacteria blended from multiple donors (and optionally one or more bacterial isolates); (4) optionally testing of the subject to determine efficacy, if an additional dose is necessary, or if the dose should be adjusted; and (5) optionally administration of a third pharmaceutical composition comprising a preparation of uncultured fecal bacteria blended from multiple donors (and optionally one or more bacterial isolates), where the multiple donors (a) comprise all donors from the second pharmaceutical composition and additional donors, (b) comprise donors of fecal bacteria not included in the second pharmaceutical composition, (c) comprise some but not all of the donors of fecal bacteria included in the second pharmaceutical composition, or comprise donors of fecal bacteria not included in the second pharmaceutical composition. In another aspect, the first, second, and third pharmaceutical compositions are administered in a different order (i.e., first, third, second; third, second, first; third, first, second; second, first, third, etc.).

In another aspect, the present disclosure provides for methods for treating a subject in need thereof with capsules containing a pharmaceutical composition comprising a preparation of uncultured fecal bacteria from a single donor. In another aspect, a capsule comprises a pharmaceutical composition comprising a preparation of uncultured fecal bacteria from multiple donors. In one aspect, a subject is administered two or more pills comprising a preparation of uncultured fecal bacteria from a single but different donor.

In one aspect, the present disclosure provides for methods for treating a subject in need thereof comprising administering a pharmaceutical composition comprising a preparation of uncultured fecal bacteria of a single donor similar to or different from a prior administration in a treatment period. In another aspect, a treatment period includes administration of a first dose comprising a pharmaceutical composition comprising a preparation of uncultured fecal bacteria of a single donor and administration of a second dose comprising a pharmaceutical composition comprising a preparation of uncultured fecal bacteria of multiple donors.

In an aspect, a preparation of uncultured fecal bacteria and one or more bacterial isolates are administered to a subject according a method described herein in the same pharmaceutical composition. In an aspect, a preparation of uncultured fecal bacteria and one or more bacterial isolates are administered to a subject according to a method described herein in different pharmaceutical compositions. In an aspect, multiple bacterial isolates are administered to a subject according to a method described herein in the same pharmaceutical composition. In an aspect, multiple bacterial isolates are administered to a subject according to a method described herein in different pharmaceutical compositions. For example, a method can comprise administering to a subject in need thereof an effective amount of a plurality of pharmaceutical compositions, e.g., two or more pharmaceutical compositions, three or more pharmaceutical compositions, four or more pharmaceutical compositions, or five or more pharmaceutical composition, as disclosed herein. The plurality of pharmaceutical compositions can be provided simultaneously or sequentially. Thus, if a subject is to be treated with, for example, a preparation of uncultured fecal bacteria and two bacterial isolates, a first composition can comprise two of the bacterial isolates and the second composition can comprise the preparation of uncultured fecal bacteria. In a different example, if a subject is to be treated with a preparation of uncultured fecal bacteria and two bacterial isolates, a first composition can comprise the preparation of uncultured fecal bacteria in combination with (or “spiked” with) a first bacterial isolate, and a second composition can comprise the second bacterial isolate. In a different example, if a subject is to be treated with a preparation of uncultured fecal bacteria and three bacterial isolates, a first composition can comprise the first bacterial isolate, a second composition can comprise the second bacterial isolate, a third composition can comprise the third bacterial isolate, and a fourth composition can comprise the preparation of uncultured fecal bacteria.

In an aspect, a method for treating one or more symptoms of ASD in a subject in need thereof comprises administering to the subject: (i) one or more bacterial isolates; (ii) a preparation of uncultured fecal bacteria; and (iii) one or more antibiotics. The different components of (i)-(iii) can be administered to the subject in any order. For example, a subject can be administered one or more antibiotics, followed by a bacterial mixture comprising both the preparation of uncultured fecal bacteria and one or more bacterial isolates. In another example, a subject can be administered one or more antibiotics, followed by a preparation of uncultured fecal bacteria, followed by one or more bacterial isolates. In another example, the subject can be administered one or more antibiotics, followed by one or more bacterial isolates, followed by a preparation of uncultured fecal bacteria. For each of the above examples, it is further understood that any given component in a method of treatment can be administered multiple times. For example, an antibiotic can be administered to the subject, followed by a preparation of uncultured fecal bacteria, followed by one or more bacterial isolates, followed by a second administration of a preparation of uncultured fecal bacteria.

In an aspect, a method for treating one or more symptoms of ASD in a subject in need thereof comprises administering to the subject: (i) one or more bacterial isolates; (ii) a preparation of uncultured fecal bacteria; and (iii) one or more prebiotics. The different components of (i)-(iii) can be administered to the subject in any order. For example, a subject can be administered one or more prebiotics, followed by a bacterial mixture comprising both a preparation of uncultured fecal bacteria and one or more bacterial isolates. In another example, a subject can be administered one or more prebiotics, followed by a preparation of uncultured fecal bacteria, followed by one or more bacterial isolates. In another example, the subject can be administered one or more prebiotics, followed by one or more bacterial isolates, followed by a preparation of uncultured fecal bacteria. In another example, the subject can be administered one or more prebiotics following administration of one or both of the one or more bacterial isolates and/or the preparation of uncultured fecal bacteria. For each of the above examples, it is further understood that any given component in a method of treatment can be administered multiple times. For example, a preparation of uncultured fecal bacteria can be administered to a subject, followed by a one or more bacterial isolates, followed by a prebiotic, followed by a second administration of the preparation of uncultured fecal bacteria.

In an aspect, a method for treating one or more symptoms of ASD in a subject in need thereof comprises administering to the subject: (i) one or more bacterial isolates; (ii) a preparation of uncultured fecal bacteria; (iii) one or more prebiotics; and (iv) one or more antibiotics. The different components of (i)-(iv) can be administered to the subject in any order. For example, a subject can be administered one or more antibiotics, followed by one or more prebiotics, followed by a bacterial mixture comprising both a preparation of uncultured fecal bacteria and one or more bacterial isolates. In another example, a subject can be administered one or more antibiotics, followed by one or more prebiotics, followed by a preparation of uncultured fecal bacteria, followed by one or more bacterial isolates. In another example, the subject can be administered one or more antibiotics, followed by one or more prebiotics, followed by one or more bacterial isolates, followed by a preparation of uncultured fecal bacteria. In another example, the prebiotic can be administered after administering one or both of the one or more bacterial isolates and/or the preparation of uncultured fecal bacteria. For each of the above examples, it is further understood that any given component in a method of treatment can be administered multiple times. For example, an antibiotic can be administered to a subject, followed by a preparation of uncultured fecal bacteria, followed by one or more bacterial isolates, followed by a prebiotic, followed by a second administration of the preparation of uncultured fecal bacteria.

In each of the above combination treatments, the duration of time between different treatments (e.g., between administration of a preparation of uncultured fecal bacteria and one or more bacterial isolates) can be at least 1 hour, at least 2 hours, at least 6 hours, at least 12 hours, at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, or greater than 8 weeks.

In one aspect, a subject being treated is a subject already with a disorder (e.g., ASD). Administration of a disclosed pharmaceutical composition to clinically, asymptomatic human subject who is genetically predisposed or prone to a disorder (e.g., ASD) is also useful in preventing the onset of clinical symptoms. A human subject genetically predisposed or prone to ASD can be a human subject having a close family member or relative exhibiting or having suffered a disorder (e.g., ASD). In another aspect, a subject being treated is a subject in which ASD is to be prevented. In another aspect, a subject being treated is predisposed or susceptible to a disorder (e.g., ASD). In another aspect, a subject being treated is a subject diagnosed as having a disorder (e.g., ASD). In one aspect, a subject being treated is a patient in need thereof.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In one aspect, a method comprises administering a pharmaceutical composition orally, by enema, or via rectal suppository. In one aspect, a pharmaceutical composition is formulated as a geltab, pill, microcapsule, capsule, or tablet. In one aspect, a pharmaceutical composition is formulated as an enteric coated capsule or microcapsule, acid-resistant capsule or microcapsule, or formulated as part of or administered together with a food, a food additive, a dairy-based product, a soy-based product or a derivative thereof, a jelly, or a yogurt. In another aspect, a pharmaceutical composition is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

Described herein are kits comprising any herein-disclosed pharmaceutical composition and instructions for use. For example, a kit can include unit dosage forms comprising one or more bacterial mixtures. Such a kit could include one or more bacterial mixtures comprising at least one of a preparation of uncultured fecal bacteria and one or more bacterial isolates and, optionally, a delivery device to administer the composition to the subject or instructions for administering the dosage to a subject via an appropriate delivery route. In some cases, the dosage form comprises any suitable form of live bacteria (fresh, frozen, lyophilized, etc.) and is formulated for administration to a human subject orally, by nasogastric tube, by colonoscopy, or anally. As described herein, dosage forms suitable for kits provided herein include, without limitation, liquid solutions, capsules, tablets, powders, granules, and lyophilized forms.

The instructions of a kit can describe, for example, dosing information of the one or more pharmaceutical compositions in the kit. As examples, the frequency of administration and dose of a composition, e.g., the number of capsules of a pharmaceutical composition to be administered at a given time, and the number of times of administration per day/week). In an aspect in which the kit comprises more than one composition (e.g., multiple bacterial mixtures or an additional pharmaceutical composition lacking a bacterial mixture), the instructions can describe the dosing of each composition. For example, one composition can be administered before another composition, e.g., sequential administration of the two pharmaceutical compositions separated by minutes, hours, days, weeks, months, or longer. Alternately, two compositions can be administered simultaneously.

In a further aspect, provided herein is use of a bacterial mixture described herein for manufacture of a medicament for treating autism spectrum disorder or for reducing the severity of one or more symptoms of autism spectrum disorder.

Disclosed herein is a method comprising selecting a human stool donor based on the abundance of a bacterial taxa (e.g., phylum, class, order, family, genus, species or strain) in a stool of the donor, and subsequently collecting a stool from the donor to be used as a source of fecal bacteria for manufacturing a preparation of uncultured fecal bacteria. For example, a stool or fecal microbiota of a potential donor can be screened for the presence or abundance of a particular taxa of interest (e.g., of the genus Lactobacillus or the species L. reuteri) using a nucleic acid hybridization-based technique such as PCR (e.g., quantitative PCR), and if the taxa is present in the stool at a level above a threshold abundance, the donor can be selected as a donor of stool for use in preparing a pharmaceutical composition comprising the taxa of interest. In an aspect, a preparation of uncultured fecal bacteria prepared from a stool of the donor (i.e. containing the taxa of interest) can be supplemented with one or more bacterial isolates (e.g., comprising the bacterial taxa of interest, such as L. reuteri) to increase the bacterial taxa in a bacterial mixture comprising the preparation of uncultured fecal bacteria.

In another aspect, prior to making a fecal donation, a stool donor can ingest one or more bacterial isolates (e.g., a member of the genus Lactobacillus, for example L. reuteri), for example in the form of one or more probiotics. In an aspect, a stool donor can ingest a bacterial isolate prior to donating a stool in order to introduce the bacterial isolate into a fecal microbiota of the donated stool, i.e. as a bacterial strain. Therefore, a bacterial isolate desirable for inclusion in a bacterial mixture of a pharmaceutical composition can be introduced into the fecal microbiota of a stool donor via ingestion of the bacterial isolate by the donor, thereby allowing the preparation of uncultured fecal bacteria from the stool that is “ready-made” with, or already includes, a bacterial strain originating from the desired bacterial isolate. A preparation of uncultured fecal bacteria from the stool of a donor that has ingested a bacterial isolate (e.g., in the form of a probiotic) can be directly incorporated into a pharmaceutical composition described herein, without adding any additional bacterial isolate to the preparation, or alternatively can be further spiked or enriched with an additional dose of the bacterial isolate. Such “pre-spiking” of a stool donor's fecal microbiota with one or more desired bacterial strains originating as bacterial isolates dosed to a stool donor can be especially advantageous where a fecal microbiota of the donor does not endogenously comprise a bacterial strain of the same taxonomic category as the bacterial isolate (e.g., phylum, class, order, family, genus or species), or does not endogenously comprise a bacterial strain having a genetic identity to the bacterial isolate that is above a threshold level (e.g., having a 16S rRNA sequence with greater than 97% identity, greater than 98% identity, greater than 99% identity, greater than 99.1% identity, greater than 99.2% identity, greater than 99.3% identity, greater than 99.4% identity, greater than 99.5% identity, greater than 99.6% identity, greater than 99.7% identity, greater than 99.8% identity, or greater than 99.9% identity to a 16S rRNA sequence of the bacterial isolate). Herein a bacterial isolate incorporated into a fecal microbiota via ingestion of the bacterial isolate by a donor of the fecal microbiota is referred to as a “bacterial strain” (i.e., originating from the ingested bacterial isolate) to distinguish it from the purified bacterial isolate existing ex vivo.

In an aspect, a donor can ingest a probiotic comprising a bacterial isolate of a taxonomic category that is not detectable, or is present at a relative abundance below a threshold abundance, in a stool of the donor prior to ingestion of the probiotic. For example, the microbiota of a stool of a donor can be screened (e.g. using a nucleic acid hybridization technique such as PCR) for the presence of a particular taxa (phylum, class, order, family, genus, species or strain) in the microbiota of the donor. If the taxa either is not found in the microbiota, or is present at a relative abundance below a threshold abundance, then the donor can be administered or ingest a probiotic comprising a bacterial isolate of that taxa.

In an aspect, a stool donor can ingest a bacterial isolate comprising Lactobacillus reuteri to increase a level of L. reuteri in a stool of the donor. A preparation of uncultured fecal bacteria that includes the strain of L. reuteri originating from the ingested bacterial isolate can then be extracted from the stool and incorporated into a pharmaceutical composition described herein. In an aspect, the preparation of uncultured fecal bacteria is supplemented with an amount of the same bacterial isolate ingested by the donor, and/or one or more additional bacterial isolates. Optionally, a fecal microbiota of the donor can be screened before and/or after ingestion of the probiotic by the donor to determine whether L. reuteri is present in the microbiota, and if so, the abundance of the L. reuteri in the fecal microbiota.

In an aspect, a duration of time between ingestion of one or more bacterial isolates by a stool donor and collection of a stool from the donor (i.e., comprising a bacterial strain originating from the one or more bacterial isolates) can vary; for example the duration can be at least 1 hour, at least 2 hours, at least 4 hours, at least 6 hours, at least 8 hours, at least 10 hours, at least 12 hours, at least 14 hours, at least 16 hours, at least 18 hours, at least 20 hours, at least 22 hours, at least 24 hours, at least 26 hours, at least 28 hours, at least 30 hours, at least 32 hours, at least 34 hours, at least 36 hours, at least 38 hours, at least 40 hours, at least 42 hours, at least 44 hours, at least 46 hours, at least 48 hours, at least 50 hours, at least 52 hours, at least 54 hours, at least 56 hours, at least 58 hours, at least 60 hours, at least 62 hours, at least 64 hours, at least 66 hours, at least 68 hours, at least 70 hours, at least 72 hours, or greater than 72 hours.

In an aspect, a donor can ingest a dose of a bacterial isolate once or multiple times to facilitate the incorporation of the bacterial isolate into a fecal microbiota of the donor as a bacterial strain. In one aspect, a dose of a bacterial isolate can be ingested by the donor at least once or twice daily for at least three consecutive days or weeks. In another aspect, a dose is ingested at least once, twice, or three times daily for a period between 1 and 16 weeks, between 2 and 16 weeks, between 3 and 16 weeks, between 4 and 16 weeks, between 5 and 16 weeks, between 6 and 16 weeks, between 7 and 16 weeks, between 8 and 16 weeks, between 10 and 16 weeks, between 12 and 16 weeks, between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

Disclosed herein is a method of manufacturing a pharmaceutical composition, the method comprising: extracting a bacterial population or community from a stool of a healthy human donor; and incorporating the extracted bacterial population or community into the pharmaceutical composition, wherein the bacterial population or community comprises a bacterial strain originating from a probiotic ingested by the healthy human donor.

Disclosed herein is a method of manufacturing a pharmaceutical composition comprising a bacterial population or community of a healthy human donor, the method comprising: receiving a stool from the donor following ingestion by the donor of a probiotic comprising a bacterial strain; extracting the bacterial population or community from the stool, wherein the bacterial population or community comprises the bacterial strain; incorporating the bacterial population or community into the pharmaceutical composition, wherein the bacterial population or community is not cultured; and wherein prior to ingestion of the probiotic by the donor, a stool of the donor did not comprise the bacterial strain.

It will be appreciated that compositions, dosage forms, and medicaments as described herein include combination pharmaceutical compositions in which one or more additional compounds or medications are added to or otherwise co-administered with a purified fecal microbiota composition.

The present specification provides for, and includes, the following embodiments:

Embodiment 1. A pharmaceutical composition comprising a bacterial mixture comprising: (i) a preparation of uncultured fecal bacteria derived from a stool of a human donor; and (ii) a bacterial isolate comprising Lactobacillus reuteri.

Embodiment 2. The pharmaceutical composition of embodiment 1, wherein the stool, the preparation of uncultured fecal bacteria, or both do not comprise L. reuteri.

Embodiment 3. The pharmaceutical composition of embodiment 1, wherein the stool, the preparation of uncultured fecal bacteria, or both do not comprise a strain of bacteria having 100% genetic identity with the bacterial isolate comprising L. reuteri.

Embodiment 4. The pharmaceutical composition of embodiment 3, wherein the preparation of uncultured fecal bacteria does not comprise a strain of bacteria having greater than 98% genetic identity with the bacterial isolate comprising L. reuteri.

Embodiment 5. The pharmaceutical composition of embodiment 3 or embodiment 4, wherein the genetic identity is determined by comparing a 16S rRNA sequence of the bacterial isolate comprising L. reuteri with 16S rRNA sequences of the preparation of uncultured fecal bacteria.

Embodiment 6. The pharmaceutical composition of any one of embodiments 1 to 5, wherein the preparation of uncultured fecal bacteria comprises L. reuteri bacteria at a relative abundance of less than 5%.

Embodiment 7. The pharmaceutical composition of any one of embodiments 1 to 6, wherein the preparation of uncultured fecal bacteria comprises L. reuteri bacteria at a relative abundance of less than 3%.

Embodiment 8. The pharmaceutical composition of any one of embodiments 1 to 7, wherein the preparation of uncultured fecal bacteria comprises L. reuteri bacteria at a relative abundance of less than 1%.

Embodiment 9. The pharmaceutical composition of any one of embodiments 1 to 8, wherein the preparation of uncultured fecal bacteria comprises L. reuteri bacteria at a relative abundance of less than 0.5%.

Embodiment 10. A pharmaceutical composition comprising a bacterial mixture comprising: (i) a preparation of uncultured fecal bacteria derived from a stool of a human donor; and (ii) a bacterial isolate comprising a species of Lactobacillus; wherein the preparation of uncultured fecal bacteria does not comprise the species of the genus Lactobacillus.

Embodiment 11. The pharmaceutical composition of embodiment 10, wherein the preparation of uncultured fecal bacteria does not comprise a bacterial strain having greater than 99% genetic identity with the bacterial isolate comprising a species of Lactobacillus.

Embodiment 12. The pharmaceutical composition of embodiment 10 or embodiment 11, wherein the preparation of uncultured fecal bacteria does not comprise a bacterial strain having greater than 97% genetic identity with the bacterial isolate comprising a species of Lactobacillus.

Embodiment 13. The pharmaceutical composition of embodiment 11 or embodiment 12, wherein the genetic identity is determined by comparing a 16S rRNA sequence of the bacterial isolate comprising a species of Lactobacillus with 16S rRNA sequences of the preparation of uncultured fecal bacteria.

Embodiment 14. The pharmaceutical composition of any one of embodiments 10 to 13, wherein the species of Lactobacillus is L. reuteri.

Embodiment 15. The pharmaceutical composition of any one of embodiments 10 to 13, wherein the species of Lactobacillus is L. plantarum.

Embodiment 16. The pharmaceutical composition of any one of embodiments 10 to 13, wherein the species of Lactobacillus is L. acidophilus.

Embodiment 17. The pharmaceutical composition of any one of embodiments 10 to 13, wherein the species of Lactobacillus is L. paracasei.

Embodiment 18. The pharmaceutical composition of any one of embodiments 10 to 13, wherein the species of Lactobacillus is L. delbrueckii subsp. bulgaricus.

Embodiment 19. The pharmaceutical composition of any one of embodiments 1 to 18, wherein the pharmaceutical composition further comprises one or more additional bacterial isolates.

Embodiment 20. The pharmaceutical composition of embodiment 19, wherein the one or more additional bacterial isolates comprises a member of the genus Bifidobacterium or the genus Streptococcus.

Embodiment 21. A pharmaceutical composition comprising a bacterial mixture comprising: (i) a preparation of uncultured fecal bacteria derived from a stool of a human donor; and (ii) a non-pathogenic bacterial isolate, wherein a relative abundance of viable cells of the bacterial isolate in the bacterial mixture is at least 10%.

Embodiment 22. The pharmaceutical composition of embodiment 21, wherein the relative abundance of viable cells of the bacterial isolate in the bacterial mixture is at least 30%.

Embodiment 23. The pharmaceutical composition of embodiment 21, wherein the relative abundance of viable cells of the bacterial isolate in the bacterial mixture is at least 45%.

Embodiment 24. The pharmaceutical composition of any one of embodiments 21 to 23, wherein the relative abundance of viable cells of the bacterial isolate in the bacterial mixture is greater than a relative abundance of viable cells of any bacterial species in the preparation of uncultured fecal bacteria.

Embodiment 25. A pharmaceutical composition comprising a bacterial mixture comprising: (i) a preparation of uncultured fecal bacteria derived from a stool of a human donor; and (ii) a non-pathogenic bacterial isolate; wherein a relative abundance of viable cells of the bacterial isolate in the bacterial mixture is greater than a relative abundance of viable cells of any bacterial strain in the preparation of uncultured fecal bacteria.

Embodiment 26. The pharmaceutical composition of embodiment 25, wherein the relative abundance of viable cells of the bacterial isolate in the bacterial mixture is greater than a relative abundance of viable cells of any bacterial species in the preparation of uncultured fecal bacteria.

Embodiment 27. The pharmaceutical composition of embodiment 25, wherein the relative abundance of viable cells of the bacterial isolate in the bacterial mixture is greater than a relative abundance of viable cells of any bacterial genus in the preparation of uncultured fecal bacteria.

Embodiment 28. The pharmaceutical composition of embodiment 25, wherein the relative abundance of viable cells of the bacterial isolate in the bacterial mixture is greater than a relative abundance of viable cells of any bacterial phylum in the preparation of uncultured fecal bacteria.

Embodiment 29. A pharmaceutical composition comprising a bacterial mixture comprising: (i) a preparation of uncultured fecal bacteria derived from a stool of a human donor; and (ii) a non-pathogenic bacterial isolate; wherein the bacterial isolate is a member of a species, wherein the bacterial isolate is the only member of the species in the bacterial mixture.

Embodiment 30. A pharmaceutical composition comprising a bacterial mixture comprising: (i) a preparation of uncultured fecal bacteria derived from a stool of a human donor; and (ii) a non-pathogenic bacterial isolate; wherein the preparation of uncultured fecal bacteria does not comprise a bacterial strain having a 16S rRNA sequence greater than 99% identical to a 16S rRNA sequence of the bacterial isolate.

Embodiment 31. The pharmaceutical composition of embodiment 30, wherein the preparation of uncultured fecal bacteria does not comprise a bacterial strain having a 16S rRNA sequence greater than 97% identical to a 16S rRNA sequence of the bacterial isolate.

Embodiment 32. A pharmaceutical composition comprising a bacterial mixture comprising: (i) a preparation of uncultured fecal bacteria derived from a stool of a human donor; and (ii) a non-pathogenic bacterial isolate; wherein the bacterial isolate engrafts in the ileum of a subject administered the composition.

Embodiment 33. The pharmaceutical composition of any one of embodiments 21 to 32, wherein the bacterial isolate comprises a member of the genus Lactobacillus.

Embodiment 34. The pharmaceutical composition of embodiment 33, wherein the member of the genus Lactobacillus is selected from the group consisting of L. reuteri, L. plantarum, L. acidophilus, L. paracasei, L. delbrueckii subsp. bulgaricus, and a combination thereof.

Embodiment 35. The pharmaceutical composition of embodiment 34, wherein the member of the genus Lactobacillus is L. reuteri.

Embodiment 36. The pharmaceutical composition of any one of embodiments 21 to 35, wherein the bacterial isolate comprises a member of the genus Bifidobacterium.

Embodiment 37. The pharmaceutical composition of embodiment 35, wherein the member of the genus Bifidobacterium is selected from the group consisting of B. breve, B. longum, B. longum, subsp. infantis, and a combination thereof.

Embodiment 38. The pharmaceutical composition of any one of embodiments 21 to 36, wherein the bacterial isolate comprises a member of the genus Streptococcus.

Embodiment 39. The pharmaceutical composition of embodiment 37, wherein the member of the genus Streptococcus is S. salivarius, subsp. thermo.

Embodiment 40. The pharmaceutical composition of any one of embodiments 21 to 39, wherein the pharmaceutical composition comprises multiple bacterial isolates.

Embodiment 41. The pharmaceutical composition of any one of embodiments 1 to 40, wherein the preparation of uncultured fecal bacteria comprises non-selected fecal bacteria.

Embodiment 42. The pharmaceutical composition of any one of embodiments 1 to 41, wherein the bacterial mixture comprises lyophilized bacteria.

Embodiment 43. The pharmaceutical composition of any one of embodiments 1 to 42, wherein the bacterial mixture comprises a cryoprotectant.

Embodiment 44. The pharmaceutical composition of any one of embodiments 1 to 43, wherein the bacterial mixture is enclosed in a capsule.

Embodiment 45. The pharmaceutical composition of embodiment 44, wherein the capsule is a delayed-release capsule.

Embodiment 46. The pharmaceutical composition of embodiment 45, wherein the delayed-release capsule is formulated to release the bacterial isolate in the ileum of a subject administered the composition.

Embodiment 47. A method of treating at least one symptom of an autism spectrum disorder (ASD) in a subject in need thereof, the method comprising administering to the subject a pharmaceutical composition of any one of embodiments 1 to 47.

Embodiment 48. A method of treating at least one symptom of an autism spectrum disorder (ASD) in a subject in need thereof, the method comprising administering to the subject (i) a pharmaceutical composition comprising a bacterial population derived from a stool of a human donor, wherein the bacterial population is not cultured; and (ii) a bacterial isolate comprising Lactobacillus reuteri.

Embodiment 49. The method of embodiment 48, wherein the pharmaceutical composition comprises the bacterial isolate.

Embodiment 50. The method of embodiment 48 or embodiment 49, wherein the bacterial population lacks L. reuteri.

Embodiment 51. A method of engrafting Lactobacillus reuteri in an intestine of a human, the method comprising administering to the human a pharmaceutical composition comprising (i) a preparation of uncultured fecal bacteria; and (ii) a bacterial isolate comprising L. reuteri; wherein a relative abundance of L. reuteri in an intestinal microbiota of the human after administering the composition is greater than a relative abundance of L. reuteri in the intestinal microbiota prior to administering the composition.

Embodiment 52. The method of embodiment 51, wherein the intestinal microbiota comprises a microbiota of the ileum.

Embodiment 53. The method of embodiment 51, wherein the intestinal microbiota comprises a fecal microbiota.

Embodiment 54. The method of any one of embodiments 51 to 53, wherein the relative abundance of L. reuteri in the intestinal microbiota of the human after administering the composition is greater than a relative abundance of L. reuteri in the intestinal microbiota after administering the bacterial isolate alone.

Embodiment 55. The method of any one of embodiments 51 to 54, wherein administering the composition treats at least one symptom of an autism spectrum disorder (ASD) in the human.

Embodiment 56. A method comprising: extracting a bacterial population from a stool of a healthy human donor; and mixing the bacterial population with a bacterial isolate comprising Lactobacillus reuteri; wherein the bacterial population is not cultured.

Embodiment 57. The method of embodiment 56, further comprising mixing the bacterial population with an additional bacterial isolate.

Embodiment 58. A method comprising: selecting a human stool donor based on an abundance of at least one member of Lactobacillus in a fecal microbiota of the donor; extracting a population of bacteria from a stool of the donor, wherein the population of bacteria comprises the at least one member of Lactobacillus; and incorporating the population of bacteria into a pharmaceutical composition, wherein the population of bacteria is not cultured.

Embodiment 59. The method of embodiment 58, wherein the method further comprises mixing the population of bacteria with a bacterial isolate comprising the at least one member of Lactobacillus.

Embodiment 60. The method of embodiment 58 or embodiment 59, wherein the at least one member of Lactobacillus is selected from the group consisting of L. reuteri, L. plantarum, L. acidophilus, L. paracasei, L. delbrueckii subsp. bulgaricus, and a combination thereof.

Embodiment 61. The method of embodiment 60, wherein the at least one member of the genus Lactobacillus is L. reuteri.

Embodiment 62. A method of manufacturing a pharmaceutical composition, the method comprising: extracting a bacterial population from a stool of a healthy human donor; and incorporating the extracted bacterial population into the pharmaceutical composition, wherein the bacterial population comprises a bacterial strain originating from a probiotic ingested by the healthy human donor.

Embodiment 63. A method of manufacturing a pharmaceutical composition comprising a bacterial population of a healthy human donor, the method comprising: receiving a stool from the donor following ingestion by the donor of a probiotic comprising a bacterial strain; extracting the bacterial population from the stool, wherein the bacterial population comprises the bacterial strain; incorporating the bacterial population into the pharmaceutical composition, wherein the bacterial population is not cultured; and wherein prior to ingestion of the probiotic by the donor, a stool of the donor did not comprise the bacterial strain.

Embodiment 64. The method of embodiment 63, further comprising determining that a stool of the donor does not comprise the bacterial strain prior to ingestion of the probiotic by the donor.

Embodiment 65. A pharmaceutical composition comprising a bacterial mixture comprising: (i) a preparation of uncultured fecal bacteria derived from a stool of a human donor; and (ii) a non-pathogenic bacterial isolate of a Lactobacillus species; wherein a relative abundance of viable cells of the Lactobacillus species in the bacterial mixture is greater than a relative abundance of viable cells of the Lactobacillus species in fecal bacteria of the stool.

Embodiment 66. The pharmaceutical composition of embodiment 65, wherein the Lactobacillus species is Lactobacillus reuteri.

Embodiment 67. A pharmaceutical composition comprising a mixture comprising: (i) a preparation of uncultured fecal bacteria derived from a stool of a human donor; and (ii) at least one, at least two, or all three of non-pathogenic microbial types selected from the group consisting of a bacterial isolate, a fungal isolate, and an archaeal isolate; wherein a relative abundance of viable cells of the at least one, at least two, or all three of non-pathogenic microbe types in the mixture is at least 10%.

Embodiment 68. A pharmaceutical composition comprising a mixture comprising: (i) a preparation of uncultured fecal bacteria derived from a stool of a human donor; and (ii) at least one, at least two, or all three of non-pathogenic microbial types selected from the group consisting of a bacterial isolate, a fungal isolate, and an archaeal isolate; wherein a relative abundance of viable cells of the at least one, at least two, or all three of non-pathogenic microbe types in the mixture is greater than a relative abundance of viable cells of any bacterial strain in the preparation of uncultured fecal bacteria.

Embodiment 69. A pharmaceutical composition comprising a mixture comprising: (i) a preparation of uncultured fecal bacteria derived from a stool of a human donor; and (ii) at least one, at least two, or all three of non-pathogenic microbial types selected from the group consisting of a bacterial isolate, a fungal isolate, and an archaeal isolate; wherein the bacterial isolate is a member of a first species, wherein the bacterial isolate is the only member of the first species in the mixture, wherein the fungal isolate is a member of a second species, wherein the fungal isolate is the only member of the second species in the mixture, wherein the archaeal isolate is a member of a third species, wherein the archaeal isolate is the only member of the third species in the mixture.

Embodiment 70. A pharmaceutical composition comprising a mixture comprising: (i) a preparation of uncultured fecal microbes derived from a stool of a human donor; and (ii) at least one, at least two, or all three of non-pathogenic microbial types selected from the group consisting of a bacterial isolate, a fungal isolate, and an archaeal isolate; wherein the preparation of uncultured fecal microbes does not comprise a bacterial strain having a 16S rRNA sequence greater than 99% identical to a 16S rRNA sequence of the bacterial isolate, wherein the preparation of uncultured fecal microbes does not comprise a fungal strain having a 16S rRNA sequence greater than 99% identical to a 16S rRNA sequence of the fungal isolate, wherein the preparation of uncultured fecal microbes does not comprise a archaeal strain having a 16S rRNA sequence greater than 99% identical to a 16S rRNA sequence of the archaeal isolate.

Embodiment 71. A pharmaceutical composition comprising a mixture comprising: (i) a preparation of uncultured fecal bacteria derived from a stool of a human donor; and (ii) at least one, at least two, or all three of non-pathogenic microbial types selected from the group consisting of a bacterial isolate, a fungal isolate, and an archaeal isolate; wherein the at least one, at least two, or all three of non-pathogenic microbial types engraft in the ileum of a subject administered the composition.

Embodiment 72. A method for treating at least one symptom of an autism spectrum disorder (ASD) in a subject in need thereof, the method comprising administering to the subject (i) a pharmaceutical composition comprising a bacterial population derived from a stool of a human donor, wherein the bacterial population is not cultured; and (ii) at least one, at least two, or all three of non-pathogenic microbial types selected from the group consisting of a bacterial isolate, a fungal isolate, and an archaeal isolate.

Embodiment 73. A method comprising: extracting a bacterial population from a stool of a healthy human donor; and mixing the bacterial population with (i) a non-pathogenic bacterial isolate and (ii) at least one, at least two, or all three of non-pathogenic microbial types selected from the group consisting of a bacterial isolate, a fungal isolate, and an archaeal isolate; wherein the bacterial population is not cultured.

Embodiment 74. A method comprising: selecting a human stool donor based on an abundance of at least one member in a fecal microbiota of the donor; extracting a population of microbes from a stool of the donor, wherein the population of microbes comprises the at least one member; and incorporating the population of microbes into a pharmaceutical composition, wherein the population of microbes is not cultured, wherein the at least one member comprises at least one, at least two, or all three of non-pathogenic microbial types selected from the group consisting of a bacterial isolate, a fungal isolate, and an archaeal isolate.

Embodiment 75. A method of manufacturing a pharmaceutical composition, the method comprising: extracting a microbial population from a stool of a healthy human donor; and incorporating the extracted microbial population into the pharmaceutical composition, wherein the microbial population comprises at least one member originating from a probiotic ingested by the healthy human donor, wherein the at least one member comprises at least one, at least two, or all three of non-pathogenic microbial types selected from the group consisting of a bacterial isolate, a fungal isolate, and an archaeal isolate.

Embodiment 76. A method of manufacturing a pharmaceutical composition comprising a microbial population of a healthy human donor, the method comprising: receiving a stool from the donor following ingestion by the donor of a probiotic comprising at least one, at least two, or all three of non-pathogenic microbial types selected from the group consisting of a bacterial isolate, a fungal isolate, and an archaeal isolate; extracting the microbial population from the stool, wherein the microbial population comprises the at least one, at least two, or all three of non-pathogenic microbial types; incorporating the microbial population into the pharmaceutical composition, wherein the microbial population is not cultured; and wherein prior to ingestion of the probiotic by the donor, a stool of the donor did not comprise microbes of the at least one, at least two, or all three of non-pathogenic microbial types.

The disclosure may be better understood by reference to the following non-limiting Examples, which are provided as exemplary of the disclosure. The following examples are presented in order to more fully illustrate the preferred aspects of the disclosure and should in no way be construed, however, as limiting the broad scope of the disclosure. Therefore, the scope of the appended claims should not be limited to the description of the aspects contained herein.

### EXAMPLES

### Example 1: Variable Relative Abundance of Lactobacillus reuteri in Human Donor Stool

Operational taxonomic units (OTUs) belonging to the genus Lactobacillus and species Lactobacillus reuteri were identified from GreenGenes (v 13_8) and SILVA (SSU r132) using taxonomic annotations included in these databases. GreenGenes and SILVA OTU tables were constructed by mapping NGS reads (sequencing performed at the Broad Institute) from 1195 samples originating in 90 stool donors and 15 run controls to GreenGenes and SILVA databases at 97% and 99% identity respectively.

FIG. 1 shows the median summed relative abundance of all Lactobacillus OTUs and only Lactobacillus reuteri OTUs in all samples from each donor. The graph shows that there is variability in the relative abundance of Lactobacillus reuteri in fecal microbiota of healthy stool donors.

### Example 2: Production of a Pharmaceutical Composition from Stool of a Donor Selected on the Basis of the Presence of L. reuteri in the Stool

Based on the results of FIG. 1, a donor is selected based on the presence of L. reuteri in the stool of the donor. A stool sample is collected from the donor, diluted with 15% trehalose and 0.05% cysteine in PBS saline, homogenized, and filtered. The filtrate is lyophilized and the lyophilized bacterial mixture is encapsulated to form a pharmaceutical composition.

### Example 3: Production of a Bacterial Mixture

A stool sample is collected from a screened, healthy human donor, diluted with 15% trehalose and 0.05% cysteine in PBS saline, homogenized, and filtered. The filtrate is subjected to lyophilization to give rise to a lyophilized preparation of uncultured fecal bacteria. A bacterial isolate comprising lyophilized Lactobacillus reuteri (ATCC PTA 6475) is obtained and mixed with the lyophilized preparation of uncultured fecal bacteria to produce a bacterial mixture comprising the L. reuteri bacterial isolate and the preparation of uncultured fecal bacteria.

### Example 4: Production of a Donor-Derived Bacterial Composition with an Assimilated Bacterial Strain of Interest

A pre-screened, healthy donor ingests a probiotic composition containing one or more desired bacterial strains (e.g., Lactobacillus). A stool sample is collected from the donor and processed to extract a bacterial population which includes a desired bacterial strain of the probiotic. Such a desired bacterial strain is either absent in the donor's fecal microbiota prior to the ingestion or at a lower level. The extracted bacterial population is then formulated into a pharmaceutical composition.

### Example 5: Immune-Stimulating Effects from a Donor-Derived Bacterial Composition Combined with an Bacterial Strain of Interest

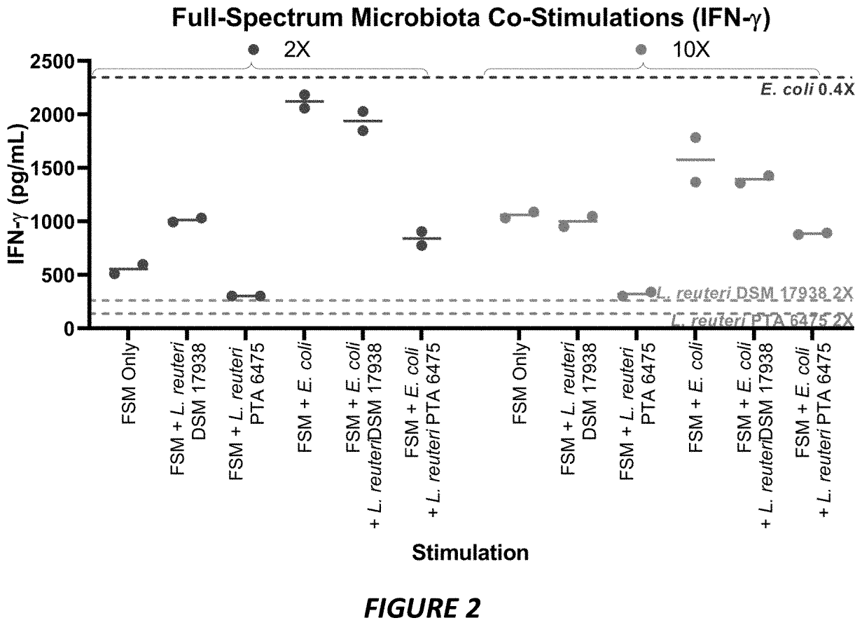

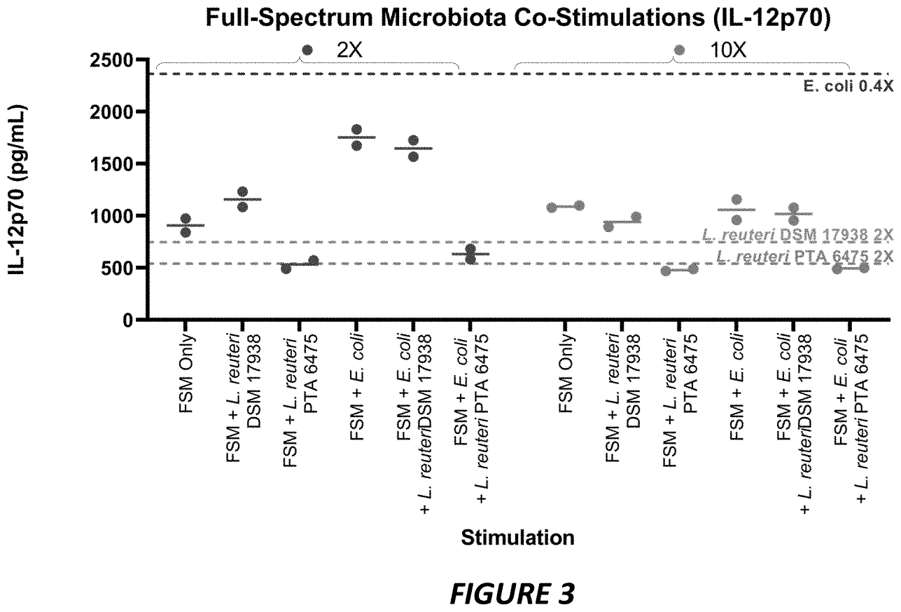

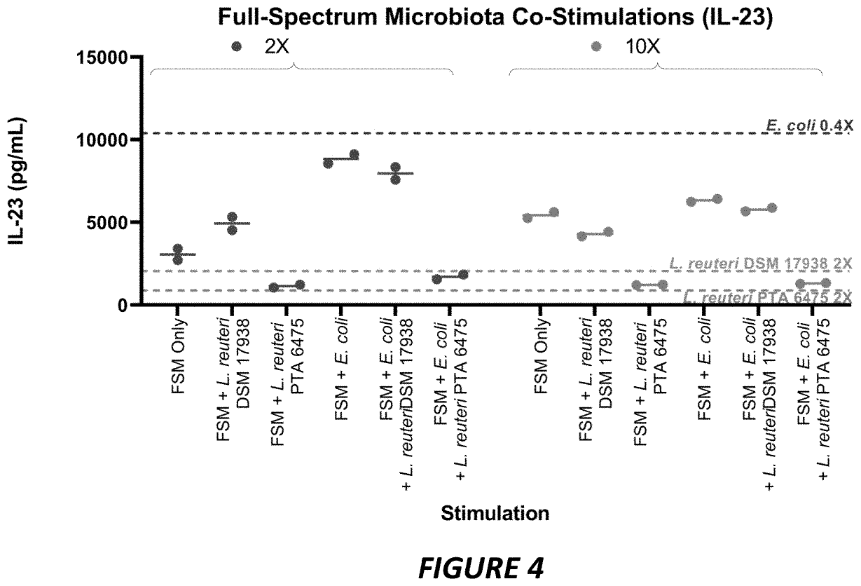

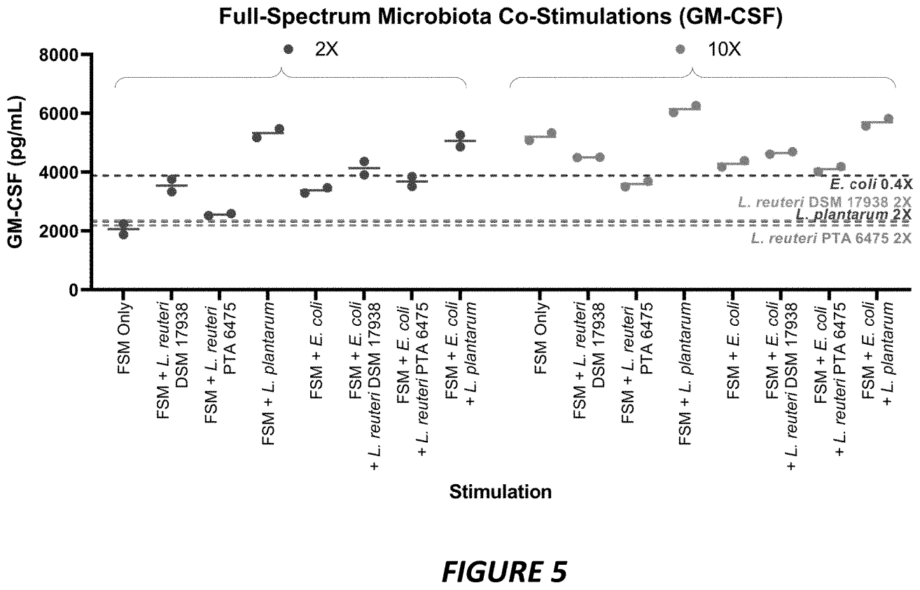

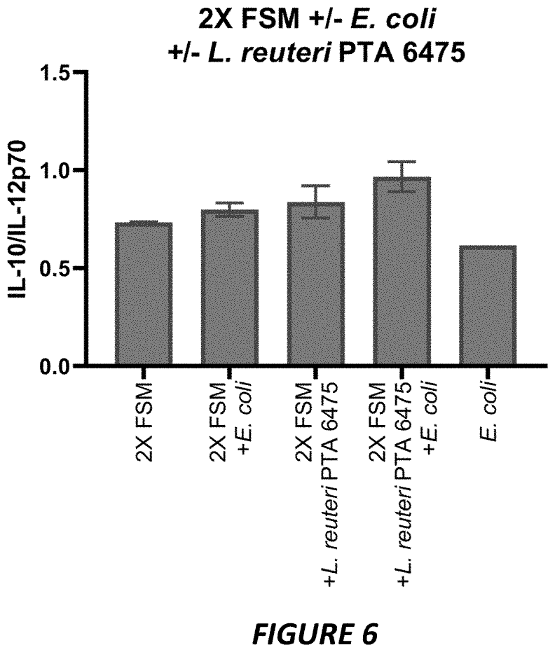

Three Lactobacillus isolates, including L. reuteri (ATCC PTA 6475), L. reuteri (DSM 17938), and L. plantarum, are cultured in liquid vBHI3 media. Cells are washed and re-suspended at 1×10⁹ CFU/mL (optionally, stored at −80° C. until the later co-culture step). Human PBMCs are seeded into 24-well plates and incubated in 5% CO₂ overnight. Each bacterial stimulation is added to the PBMCs and co-cultured for 24 hours. For all single and coo-stimulations, Lactobacillus strains are added at a multiplicity of infection (MOI) of 2× relative to the number of PBMCs, the non-selected donor-derived fecal microbiota (FSM) component is added at an MOI of either 2× or 10×, and E. coli is added at an MOI of 0.4×. The co-culture supernatants are then collected and cytokine levels (of IL-10, IL-12p70, IFN-gamma, GM-CSF, TNF-alpha, and IL-23) are quantified by Luminex. The combination of FSM with Lactobacillus strains L. reuteri (DSM 17938) (FIGS. 2 to 5), L. plantarum (FIG. 5) and L. reuteri (ATCC PTA 6475) (FIGS. 2 to 6) resulted in downregulation of pro-inflammatory cytokines (FIGS. 2 to 4), upregulation of an anti-inflammatory cytokine (FIG. 5) and an increase in the IL-10/IL-12 ratio in response to an inflammatory stimulus (E. coli). These results show that a bacterial mixture comprising both a preparation of uncultured fecal bacteria and a Lactobacillus bacterial isolate can be administered to cultured human cells to promote an anti-inflammatory immune profile.

### Example 6: Treatment of ASD

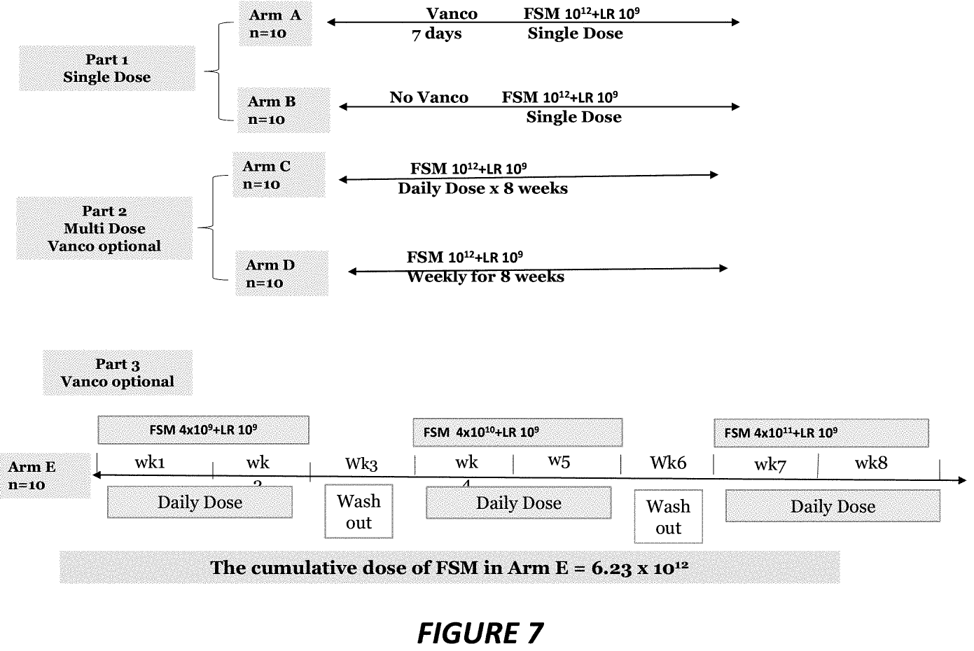

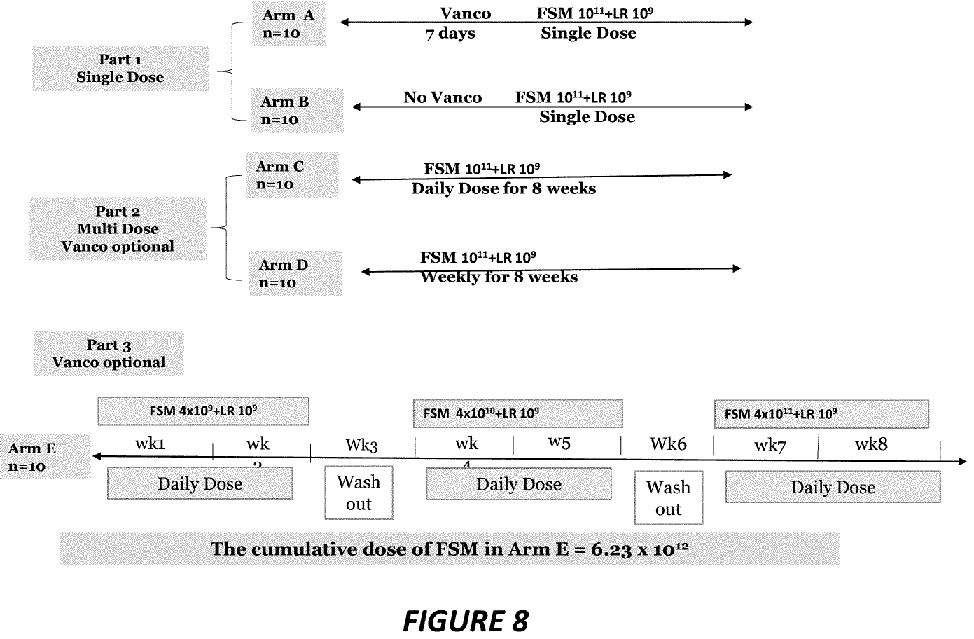

ASD patients are divided into groups and receive different treatment regimens which differ in pretreatment (e.g., antibiotic (Vancomycin) or bowel preparation), treatment length (single dose or multi-dose), dose (either individual or overall dose), or dosing frequency (daily or weekly) (FIGS. 7 and 8). Fecal materials from various donors are used to produce bacterial compositions used at a given dose. Bacterial compositions used at different dose levels can be from fecal materials from the same donor. Patient symptoms are observed and clinical examination is performed before, during and post treatment. Pre, during and post-treatment DNA metagenomics of patient fecal samples are also carried out to determine bacterial engraftment.

1. A pharmaceutical composition comprising a bacterial mixture comprising: (i) a preparation of uncultured fecal bacteria derived from a stool of a human donor; and (ii) a bacterial isolate comprising Lactobacillus reuteri.

2. A pharmaceutical composition comprising a bacterial mixture comprising: (i) a preparation of uncultured fecal bacteria derived from a stool of a human donor; and (ii) a bacterial isolate comprising a species of Lactobacillus; wherein the preparation of uncultured fecal bacteria does not comprise the species of the genus Lactobacillus.

3. A pharmaceutical composition comprising a bacterial mixture comprising: (i) a preparation of uncultured fecal bacteria derived from a stool of a human donor; and (ii) a non-pathogenic bacterial isolate, wherein a relative abundance of viable cells of the bacterial isolate in the bacterial mixture is at least 10%.

4-25. (canceled)
